(19) United States
(12) Patent Application Publication
(10) Pub. No.: US 2025/0228971 A1
(43) Pub. Date: Jul. 17, 2025

(54) DESIGN AND CONSTRUCTION OF EVOLUTIONARY-GUIDED "SELECTION GENE DRIVE" THERAPY

(71) Applicant: THE PENN STATE RESEARCH FOUNDATION, University Park, PA (US)

(72) Inventors: Justin Robert PRITCHARD, University Park, PA (US); Scott Matthew LEIGHOW, University Park, PA (US)

(21) Appl. No.: 18/854,783

(22) PCT Filed: Apr. 6, 2023
(86) PCT No.: PCT/US23/65460

§ 371 Date: Oct. 7, 2024

Related U.S. Application Data
(60) Provisional application No. 63/328,102, filed on Apr. 6, 2022.
(60) Provisional application No. 63/454,946, filed on Mar. 27, 2023.

(51) Int. Cl. A61K 48/00; A61K 38/16; A61K 38/44; A61K 38/50; A61K 38/52; A61P 35/00

(52) U.S. Cl. CPC A61K 48/005; A61K 38/164; A61K 38/44; A61K 38/50; A61K 38/52; A61P 35/00; C12Y 305/04001; C12Y 502/01008

(57) ABSTRACT

The present disclosure relates compositions, systems, and methods for treating, inhibiting, decreasing, reducing, ameliorating, and/or preventing a cancer or a proliferative disease using a selection gene drive therapy.

### RELATED APPLICATION

This PCT application claims priority to, and the benefit of, U.S. Provisional Patent Application Nos. 63/328,102, filed Apr. 6, 2022, entitled “CONSTRUCTION OF EVOLUTIONARY-GUIDED ‘SELECTION GENE DRIVE’ THERAPY,” and 63/454,946, filed Mar. 27, 2022, entitled “DESIGN AND CONSTRUCTION OF EVOLUTIONARY-GUIDED ‘SELECTION GENE DRIVE’ THERAPY,” which is incorporated by reference herein in its entirety.

### GOVERNMENT SUPPORT

This invention was made with Government Support under Grant No. 1R21EB026617-01A1 and Grant No. U01 CA265709-01 awarded by National Institutes of Health. The Government has certain right in the invention.

### REFERENCE TO SEQUENCE LISTING

The sequence listing submitted on Apr. 6, 2023, as an XML file entitled “11196-082WO1_Sequence_Listing.xml” created on Apr. 6, 2023, and having a file size of 13,099 bytes is hereby incorporated by reference pursuant to 37 C.F.R. § 1.52 (e) (5).

### FIELD

The present disclosure relates compositions, systems, and methods for treating, inhibiting, decreasing, reducing, ameliorating, and/or preventing a cancer or a proliferative disease using a selection gene drive therapy.

### BACKGROUND

Tyrosine kinase inhibitors, like crizotinib, erlotinib, alectinib and Osimertinib, are targeted cancer therapies that identify and attack various types of cancer cells while causing minimal damage to normal, healthy cells. These inhibitors also target ALK fusions and EGFR mutations in cancers, such as NSCLC, and provide impressive objective responses in biomarker defined late stage cancer patients. The clinical success of ALK and EGFR therapies has led to investigations for other activated tyrosine kinases in NSCLC and other cancers.

However, despite efforts to develop other tyrosine kinase inhibitors, tumors eventually acquire drug resistance. Following initial responses to crizotinib and erlotinib, ALK and EGFR driven NSCLC's return as drug resistant tumors with a worse prognosis and fewer treatment options. When drug resistance occurs in the tyrosine kinase, next generation kinase inhibitors like alectinib and osimertinib have impressive response rates in these refractory patients, but the responses are once again short lived (10⁻¹²mths) and resistance re-develops.

Given the evolution of drug resistance to inhibitors, there is need to address the aforementioned problems mentioned above by developing therapies to prevent drug resistance.

### SUMMARY

The present invention relates to nucleic acids comprising a guided selection gene drive system and methods for the manufacture and use thereof.

In one aspect, disclosed herein are nucleic acid compositions comprising a fitness benefit gene or compound, a fitness cost gene (such as, for example, a cytosine deaminase gene, a NADPH nitroreductase gene, or a diptheria toxin gene), and a promoter within a pharmaceutically acceptable carrier, wherein the fitness benefit gene or compound comprises a dimerization domain gene operably linked to a drug resistant gene. In some aspects, the dimerization protein (such as for example, FK506-binding protein 12 (FKBP12)) is fused to the drug resistance receptor.

In some embodiments, the fitness benefit molecule comprises a resistance gene, metabolite, a growth factor, a cytokine, a supplement, or a biomolecule thereof. In some embodiments, the fitness cost gene is a suicide gene.

In some embodiments, the fitness benefit gene is 2 or more kilobases in length. In some embodiments, the fitness benefit gene is 2, 3, 4, 5, 6, 7, 8, 9, 10, or more kilobases in length. In some embodiments, the fitness cost gene is at least 0.25 kilobases (kb) in length. In some embodiments the suicide gene is at least 0.25, 0.5, 0.75, 1.0, 1.1, 1.2, 1.3, 1.4, 1.5, 1.6, 1.7, 1.8, 1.9, 2.0, 2.1, 2.2, 2.3, 2.4, or 2.5 kilobases (kb) in length.

In some embodiments, the fitness cost gene is located downstream of the resistance gene.

In some embodiments, the dimerization domain gene encodes a dimerizing protein. In some embodiments, the drug resistant gene encodes a drug resistant receptor. In some embodiments, the dimerizing protein is fused to the drug resistant receptor. In some embodiments, the drug resistant receptor is a drug resistant tyrosine kinase receptor.

In some embodiments, the suicide gene encodes a suicide enzyme. In some embodiments, the suicide gene is a cytosine deaminase gene, a NADPH nitroreductase gene, or a diptheria toxin gene. In some embodiments, the fitness benefit gene or compound, the fitness cost gene, and promoter are encoded on a retroviral vector including, but not limited to a lentiviral vector.

Also disclosed herein are cells comprising the nucleic acid compositions of any preceding aspect.

In one aspect, disclosed herein are gene selection drive systems comprising the nucleic acid composition of any preceding aspect, wherein said system is activated in a cell population comprising a dimerizer (such as, for example, a peptide, polypeptide, or small molecule including, but not limited to FK506-binding protein 12 (FKBP12) peptide or a dihydrofolate reductase (DHFR) polypeptide) and a therapeutic compound (such as, for example, an anti-cancer therapeutic including, but not limited to prodrugs of anti-cancer therapeutics). For example, disclosed herein are gene selection drive systems comprising a fitness benefit molecule, a fitness cost gene (such as, for example, a cytosine deaminase gene, a NADPH nitroreductase gene, or a diptheria toxin gene), and a promoter within a pharmaceutically acceptable carrier, wherein the fitness benefit molecule comprises a dimerization domain gene operably linked to a drug resistant gene. In some aspects, the dimerization protein is fused to the drug resistance receptor. In one aspect, the nucleic acid can be encoded in a cell, including, but not limited to a cell population (such as, for example, a cell population comprising a first, second, and/or third cell).

In some embodiments, the dimerizer and the therapeutic compound are administered simultaneously or individually to the cell population.

In some embodiments, the dimerizer interacts with one or more dimerizing proteins fused to the drug resistant receptor to induce drug resistance in the first cell, wherein the therapeutic compound kills the second cell, and wherein the second cell comprises an innate drug resistance.

In embodiments, the suicide enzyme is expressed in the first cell and the third cell. In embodiments, the suicide enzyme is expressed in response to a physical stimulus (such as for example, increased population of cells), chemical stimulus (such as, for example, a doxycycline compound or a tetracycline compound), or a genetic stimulus, (such as, for example, any cell specific promotor or any tumor specific promoter).

In some embodiments, the suicide enzyme converts a prodrug into an active drug. In embodiments, the active drug kills the first cell and third cell or a residual cell not comprising the system.

In some embodiments, the dimerizer is a peptide, polypeptide, or a small molecule. In some embodiments, the dimerizer is a FK506-binding protein12 (FKBP12) peptide. In some embodiments, the active drug is a chemotherapy drug.

In one aspect, disclosed herein are cells comprising the gene selection drive system or nucleic acid composition of any preceding aspect.

In one aspect, disclosed herein are methods of treating, inhibiting, decreasing, reducing, ameliorating, and/or preventing a cancer and/or metastasis in a subject in need thereof, the method comprising administering to the subject the gene selection drive system or nucleic acid composition of any preceding aspect. For example, disclosed herein are methods of treating, inhibiting, decreasing, reducing, ameliorating, and/or preventing a cancer and/or metastasis in a subject in need thereof, the method comprising administering to the subject a gene selection drive system or a nucleic acid composition comprising a fitness benefit molecule, or a fitness cost gene (such as, for example, a cytosine deaminase gene, a NADPH nitroreductase gene, or a diptheria toxin gene), and a promoter within a pharmaceutically acceptable carrier, wherein the fitness benefit molecule comprises a dimerization domain operably linked to a drug resistant target gene. In some embodiments, the system is activated in a tumor of the subject when a dimerizer and a therapeutic compound are further administered simultaneously or individually.

In some embodiments, the one or more dimerizing domain is fused to a drug resistant receptor to induce drug resistance in the tumor.

In some embodiments, the fitness benefit molecule promotes cell growth in the subject. In some embodiments, the fitness cost gene encodes a suicide enzyme whereby said suicide enzyme converts a prodrug into an active chemotherapeutic drug. In some embodiments, the fitness cost gene is a cytosine deaminase gene, a NADPH nitroreductase gene, or a diptheria toxin gene.

In some embodiments, the dimerizer is a peptide, polypeptide, or small molecule. In some embodiments, the dimerizer is a FK506-binding protein 12 (FKBP12) peptide.

In some embodiments, the therapeutic compound and the active chemotherapeutic drug kill at least 80% of cancer cells in the tumor. In some embodiments, the fitness cost gene kills the remaining 1-20% of cancer cells in the tumor.

In some embodiments, the pharmaceutically acceptable carrier is a retroviral vector including, but not limited to a lentiviral vector. In some embodiments, the subject is a human.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1A, 1B, 1C, 1D, 1E, 1F, 1G, 1H, and 1I show the compartmental and agent-based stochastic models of disease evolution to establish criteria for gene drive design. FIG. 1A shows a schematic of population dynamics for a tumor undergoing sequential monotherapy. FIG. 1B shows a schematic of population dynamics for a “forward engineering” approach to cancer therapy. An engineered population is selected for during the Switch 1 phase of treatment. Then, a suicide gene with a bystander effect is used to eliminate engineered and resistant cells during the Switch 2 phase of treatment. FIG. 1C shows the schematics for Switch 1 and Switch 2 activity. Under Switch 1 (left), targeted therapy is effective against sensitive cells (blue), but not resistant cells (red). Additionally, engineered gene drive cells (green) are rescued from therapeutic killing by Switch 1 function. Under Switch 2 (right), gene drive cells activate a prodrug with diffusible activity. The activated metabolite targets gene drive cells and neighboring unmodified cells via a bystander effect. FIG. 1D shows the map of mutational pathways (i.e. points of potential system failure) included in the compartmental dynamic model. Mutational events include loss of the gene drive (no Switch 2 activity), resistance to targeted therapy (constitutive Switch 1 activity among gene drive cells, or other mutations among unmodified cells), and resistance to the Switch 2 activated prodrug. FIG. 1E shows the trajectory for one simulation of the compartmental model. Tumor detection size M=10⁹ cells; mutation rate μ=10⁻⁸; infection efficiency q=5%; net growth rate of gene drive cells ggd=0.01 (equal fitness to resistant cells). FIG. 1F shows the summary of parameter sweep for compartmental model. Initial gene drive frequency (q) and net growth rate of gene drive cells (g_{gd}) are allowed to vary. Net growth rate is shown as proportion relative to native resistant populations. Each parameter set is the frequency of eradication for 48 independent simulations. FIG. 1G shows the example initial condition for spatial agent-based model with small dispersion value (γ). FIG. 1H shows the example initial condition for a spatial agent-based model with high dispersion value (γ). FIG. 1I shows the summary of parameter sweep for spatial ABM. Bystander distance (i.e. kill radius, ρ) and dispersion parameter (γ) are allowed to vary. Dispersion represented as proportion of theoretical maximum inter-cell gene drive distance. Each parameter set is the frequency of eradication for 25 independent simulations.

FIG. 2 shows the microenvironmental impact on drug sensitivity. Drug responses tested for tumor cells grown in standard monoculture, or in co-culture with primary cancer associated fibroblasts (CAFs). The heatmap shows the degree to which drug sensitivity is changed by the presence of CAFs. Data shown for 42 common conventional or targeted chemotherapies tested in combination with 16 different primary CAF isolates.

FIG. 3 shows the comparison between a mock gene drive (left) vs a prototype dual-switch selection drive (right). The dual-switch drive engineers the evolutionary dynamics of an EGFR L858R transformed cell population in vitro. Sensitive EGFR-L858R+ transformed cells are killed by erlotinib in both plots (blue line). In the absence of a gene drive, pre-existing resistant clones (L858R/T790M) (red line) that were spiked into both populations only grows out on the left. A gene drive population (green line) whose resistance is transiently induced by rimiducid (switch 1) inhibits the growth of pre-existing resistance to the targeted therapy erlotinib (red line). The gene drive then kills the resistant population when switch 2 is induced by adding 5-FC. Rimiducid is removed to inactivate switch 1 driven drug resistance in the gene drive cell. Erlotinib is maintained throughout the experiment, effectively creating a localized combination therapy with a higher potential for a therapeutic window when switch 2 is turned on. The inset shows a close-up of the dynamics of the killing of the low-level resistance population in the gene drive containing cultures.

FIGS. 4A, 4B, 4C, and 4D shows the synthetic PEG-based lung hydrogel. FIG. 4A shows the stiffness quantification. FIG. 4B shows the integrin binding and MMP degradable proteins in real lung tissue using several mechanical characterization techniques, quantitative mass spectrometry, and literature mining, and synthetic representations by tuning hydrogel (FIG. 4C) crosslinking and incorporating different bio-functional peptides into a synthetic matrix. FIG. 4D shows that the hydrogel is coupled with 10 mono-functional integrin binding peptides (RGD, YSMKKTTMKIIPFNRLTIG (SEQ ID NO: 1), GPR, DGEA (SEQ ID NO: 2), PHSRN-RGD (SEQ ID NO: 3), LRE, GROGER (SEQ ID NO: 4), IKVAV (SEQ ID NO: 5), GRKRK (SEQ ID NO: 6), or FYFDLR (SEQ ID NO: 7)) and crosslinked with 7 di-functional MMP degradable peptides (IPVS-LRSG (SEQ ID NO: 8), RPFS-MIMG (SEQ ID NO: 9), VPLS-LTMG (SEQ ID NO: 10), VPLS-LYSG (SEQ ID NO: 11), GPLG-LWAR (SEQ ID NO: 12), IPES-LRAG (SEQ ID NO: 13), or VPMS-MRGG (SEQ ID NO: 14)) and a 4-arm PEG crosslinked with a Michael-addition reaction.

FIGS. 5A, 5B, and 5C shows the switch 1 and switch 2 modeling. FIG. 5A shows switch 1, (top) a schematic of a dimerization dependent drug resistant kinase that can be induced to create a selection drive, (middle) Dimerization dependent resistance to gefitinib of engineered Ba/F3 cells (in green) relative to EGFR L858R negative controls (blue) and T790M gefitinib resistant controls (red) as measured by cell titer glo. (bottom) Dimerization dependent activation of p-ERK1/2 in cells transfected with switch 1. FIG. 5B shows switch 2 (top) A schematic of suicide gene mediated prodrug conversion. (bottom). Cells harboring NfsA (a suicide gene) were mixed with unmodified cells and treated with the prodrug CB1954. The bowing UP of the kill line represents a bystander effect. FIG. 5C shows the modeling (top) Sensitive(S) and Gene Drive cells (G) can mutate to create resistance to targeted therapy (R,Q,C,D in Red). D,M can result from loss of the gene drive (green arrows) and/or activated prodrug resistance. A system of 8 Birth-Death-Mutation ODEs were parameterized with a range of parameters from the literature. i.e., S→G bolus infection efficiency (varies from 1/1000→ 1/10), mutation rates were varied (10⁻⁷→10⁻⁹ muts/division), A variety of birth and death rates were parameterized. Simulations utilized Gompertzian and Logistic equations to examine sensitivity to the exact birth function given conflicting clinical data showing both can fit patient data well. Carrying capacities varied 10¹²⁻¹⁴. The system was simulated stochastically-(bottom). Counterintuitively, parameter sets, and simulations show that the ability to eradicate a population with a dual-switch gene drive is highly dependent on the fitness provided by switch 1.

FIGS. 6A, 6B, and 6C show the validation of switch 1 causing inducible drug resistance in EGFR mutant PC9 cell line from an NSCLC. Addition of dimerizer causes inducible drug resistance.

FIG. 7 shows the biphasic dose response curve of cellular growth to switch 1 is predicted from the biphasic kinetic results from a theoretical analysis. Only the empirical result is shown here. The current limits are highlighted by the curve and the design goal is shaded in blue.

FIGS. 8A and 8B shows cell lines responses to switch 2. FIG. 8A shows the demonstration of the bystander effect in TPC1 cells harboring the Cytosine Deaminase (CD) switch 2 construct that are highly sensitive to this particular switch 2 design. A completely purple upper left would be an ideal bystander effect. FIG. 8B shows the dose response curves reveal cell line specific differential sensitivity for cancer cell lines harboring switch 2 (dark) or control (light).

FIG. 9A, 9B, 9C, 9D, 9E, 9F, 9G, 9H, 9I, 9J, 9K, and 9L show the modular motifs of genetic switches demonstrate inducible fitness benefits and shared fitness costs. FIG. 9A shows the schematic of modular dual-switch design. Both genetic switches can ultimately be integrated into a single genetic circuit. FIG. 9B shows the schematic of Switch 1 vEGFRerl design. A T790M mutation in the EGFR kinase domain confers resistance to erlotinib activity (left). An inducible FKBP12-EGFR fusion protein controllably induces EGFR signaling (right). A T790M resistance mutation in the EGFR kinase domain (S1 vEGFRₑᵣₗ) rescues signaling in the presence of erlotinib. Dimerization is induced by a ligand (dimerizer) AP2018731. FIG. 9C shows the switch 1 activity in S1 vEGFRₑᵣₗ BaF3 tumors in vivo. BaF3 cells transduced with S1 vEGFRₑᵣₗ require signaling through the synthetic gene in the absence of IL-3, and are thus expected to be dimerizer dependent. These cells were grafted in the flanks of mice and treated once daily with the dimerizer dose shown. Tumor volumes were measured after 12 days. Mean and standard error are shown for six tumors per treatment arm. FIG. 9D shows the switch 1 confers inducible erlotinib resistance in S1 vEGFRₑᵣₗ BaF3 cells in vitro. EGFR+ BaF3 cells were transduced with S1 vEGFRₑᵣₗ and treated with a range of erlotinib concentrations in the presence (orange) or absence (gray) of dimerizer (abbreviated Dim). Data for sensitive (EGFR L858R; blue) and resistant (EGFR L858R/T790M; red) BaF3 cells are also shown. FIG. 9E shows the switch 1 activity as measured by western blot in EGFR+PC9 cells. Sensitive (wild-type; blue), resistant (EGFR L858R/T790M; red), and gene drive (S1 vEGFRₑᵣₗ; orange) PC9 cells were treated with erlotinib and/or dimerizer. FIG. 9F shows the schematic of Switch 2 vCyD design. The enzyme cytosine deaminase converts the inert prodrug 5-FC into its activated form, 5-FU. 5-FU is diffusible; this bystander activity enables gene drive cells (green) to kill sensitive (blue) and TKI-resistant (red) cells. FIG. 9G shows the switch 2 activity in S2 vCyD BaF3 cells in vitro. EGFR+ BaF3 cells were transduced with S2 vCyD (light purple) or a control construct (wild-type; blue) and treated with a range of 5-FC concentrations. Data for control cells treated with 5-FU (the activated form of the drug; dark purple) are also shown. FIG. 9H shows the switch 2 activity in S2 vCyD BaF3 cells in vivo. EGFR+ BaF3 cells expressing S2 vCyD were grafted in mice. Upon tumor establishment, mice were dosed once daily with 5-FC (dark purple) or vehicle (light purple). Data for EGFR+ BaF3 tumors not expressing S2 vCyD (wild-type; blue) and treated with 5-FC are also shown. FIG. 9I shows the switch 2 bystander activity for S2 vCyD BaF3 cells. Mixed populations of wild-type and S2 vCyD BaF3 cells were treated with 1 mM 5-FC. The relative drug effect for pure S2 vCyD populations (gray, upper right) and pure wild-type populations (gray, lower left) are shown. In the absence of a bystander effect, the drug effect will be restricted to S2+ cells in mixed populations (gray diagonal line). Observed drug effect (purple line) is higher than the null “no bystander effect” line, showing strong bystander activity. Relative drug effect is defined as one minus relative viability. FIG. 9J shows the schematic of Switch 2 vCD19 design. CD8+ T cells (yellow) target antigen-positive cancer cells (purple) via a bispecific T cell engager. In addition, off-target immune activity kills nearby antigen-negative cancer cells (blue). FIG. 9K shows the immune bystander activity in S2 vCD19 PC9 cells. CD19+ and wild-type (CD19-) PC9 cells were co-cultured at 1:1 ratio. The CD19 bispecific T cell engager blinatumomab was added, as well as T cells at various effector: target ratios as shown. After 48 hours, cells were stained and analyzed by flow cytometry to measure CD19+ (purple) and CD19-(blue) cell viability.

FIG. 10 shows the spatial competition and microenvironmental drug resistance can delay the outgrowth of strong genetic drug resistance in a gene drive. This was preliminarily explored as 2 “cases”. Case 1: In a spatially constrained tumor (either through the properties of the ECM, or competitive population dynamics with short migration distances) small variations in local birth and death parameters in a particular microenvironmental niche can outcompete strong genetic resistance to a drug. This means that the microenvironment might delay the outgrowth of switch 1 constructs. Case 2: On the other hand, as spatial competition decreases, the gene drive can outcompete the heterogeneous cells in the local microenvironment. The top panel shows the results of an ABM incorporating strong genetic drug resistance, varying degrees of spatial competition and varying degrees of microenvironmental resistance driven by cancer associated fibroblast (CAF) infiltration. The bottom panel shows an example image of an ABM.

FIG. 11 shows the tumor cell line spheroids and patient-derived organoids in PEG hydrogels. This figure demonstrates the organoid capability of the UMASS portion of our team. A highly proliferative cell line positive control (MDA-MB-231) and patient-derived organoids was cultured in both Matrigel and PEG bone marrow gels. The 231 cells are highly proliferative in both environments whereas the patient cells are unsurprisingly minimally proliferative. Ki67-positive cells in the bone marrow gels shows that these patient cells can be cultured in 3D hydrogels. Scale bar=50 um.

FIGS. 12A, 12B, and 12C show that the selection gene drives are robust to diverse forms of resistance.

FIGS. 13A, 13B, 13C, 13D, 13E, 13F, and 13G show that the dual-switch selection gene drives demonstrate evolutionary control. FIG. 13A shows the schematic plasmid map of single lentiviral construct harboring Switch 1 (S1 vEGFRₑᵣₗ) and Switch 2 (S2 vCyD). FIG. 13B shows the BaF3 cells were stably transduced with EGFR L858R (erlotinib-sensitive; shown in blue), EGFR L858R/T790M (erlotinib-resistant and mCherry+; shown in red) or the dual-switch S1vEGFRₑᵣₗ-S2vCyD construct (GFP+; shown in green). Cells were pooled at 94.5% sensitive, 5% gene drive, and 0.5% resistant and treated with erlotinib and dimerizer. Pooled populations were analyzed by flow cytometry every two days up to 30 days. Upon outgrowth of gene drive cells, the mixed population was treated with erlotinib and 5-FC. FIGS. 13C and 13D show the functionality of complete S1vEGFRer-S2vCyD gene drive in BaF3 cells. Sensitive (blue) and resistant (red) cells were pooled without (FIG. 13C) and with (FIG. 13D) gene drive cells (green). Blue, orange, and purple arrows indicate erlotinib, dimerizer, and 5-FC treatment, respectively. Population dynamics for resistant cells are shown in the inset of (FIG. 13D). FIG. 13E shows the functionality of complete S1vEGFRer-S2vCyD gene drive for various initial frequencies. Mixed populations were seeded with the same total cell number, but the gene drive abundance varied (0.01-10%). Resistant cells were spiked in at a constant 0.1%. Blue, orange, and purple arrows indicate erlotinib, dimerizer, and 5-FC treatment, respectively. FIGS. 13F and 13G shows the functionality of SlvEGFRer-S2vCyD gene drive in BaF3 tumors in vivo. Mixed populations of EGFR+ BaF3s were prepared-0.5% resistant in (FIG. 13F) or 0.5% resistant plus 5% gene drive in (FIG. 13G)- and grafted in mice. Mice were treated once daily with erlotinib (blue arrow) and dimerizer (orange arrow) or 5-FC (purple arrow).

FIGS. 14A, 14B, 14C, 14D, 14E, 14F, and 14G show the dual-switch selection gene drives eliminate pre-existing resistance in an NSCLC model. FIG. 14A shows a schematic plasmid map of single lentiviral construct harboring Switch 1 (vEGFRosi) and Switch 2 (vCyD). FIG. 14B shows the EGFR+PC9 cells (osimertinib-sensitive; shown in blue) were stably transduced with EGFR L858R/C797S (osimertinib-resistant and mCherry+; shown in red) or the dual-switch S1vEGFRₒₛᵢ-S2vCyD construct (GFP+; shown in green). Cells were pooled at 94.5% sensitive, 5% gene drive, and 0.5% resistant and treated with osimertinib and dimerizer. Pooled populations were analyzed by flow cytometry every two days up to 30 days. Upon outgrowth of gene drive cells, the mixed population was treated with osimertinib and 5-FC. FIGS. 14C and 14D shows the functionality of complete S1vEGFRₒₛᵢ-S2vCyD gene drive in PC9 cells. Sensitive (blue) and resistant (red) cells were pooled without (FIG. 14C) and with (FIG. 14D) gene drive cells (green). Blue, orange, and purple arrows indicate osimertinib, dimerizer, and 5-FC treatment, respectively. Population dynamics for resistant cells are shown in the inset of (FIG. 14D). FIG. 14E shows the schematic of TKI resistance granted by activation of bypass oncogenes. Potential resistance mechanisms include mutations in alternative receptor tyrosine kinases, downstream effectors, or other signaling molecules that impinge upon these downstream effectors. FIG. 14F shows the resistance conferred by activated bypass oncogenes in PC9 cells. PC9 cells were transduced with a panel of parallel and downstream effectors and treated with 100 nM osimertinib. FIG. 14G shows the functionality of a complete gene drive system against various spiked-in bypass resistance populations. PC9 cells were pooled at 94.5% sensitive (wild-type; blue), 5% gene drive (S1vEGFRₒₛᵢ-S2vCyD; green), and 0.5% resistant (various oncogenes; red). Mixed populations were treated with osimertinib (blue arrow) and dimerizer (orange arrow) or 5-FC (purple arrow). Cells were analyzed by flow cytometry every two days up to 40 days.

FIGS. 15A, 15B, and 15C show the switch 1 using an inducible EGFR gene drive construct in BaF3 cells shows induced growth rate and tumor growth upon addition of dimerizer. FIG. 18A shows induced in vitro growth rate. FIG. 18B shows induced in vivo tumor growth. FIG. 18C shows that loss of the dimerizer allows control of the selection gene drive.

FIG. 16 shows that inducing the suicide gene has a wide therapeutic window in treating drug resistant cancers.

FIGS. 17A and 17B show the selection gene drive functions. FIG. 17A shows switch 1 activity. FIG. 17B shows selection gene drives functions in various cancer types and targets.

FIG. 18A, 18B, 18C, 18D, 18E, 18F, 18G, and 18H show the selection gene drives are robust to diverse forms of resistance in cis and in trans. FIG. 18A shows the schematic of EGFR single-site variant library. All codons spanning G719-H870 in the EGFR kinase domain (L858R background) were mutated for all possible amino acid substitutions. The final library is composed of 2,717 EGFR variants. FIG. 18B shows that PC9 cells were transduced with the lentiviral EGFR variant library and pooled with GFP+ gene drive cells (S1vEGFRₒₛᵢ-S2vCyD; 5% spike-in). Pooled populations were treated with osimertinib and dimerizer. Cells were analyzed by flow cytometry every two days up to 33 days. Upon outgrowth of the gene drive population, cells were treated with osimertinib and 5-FC. FIG. 18C shows the variant allele frequencies of the EGFR variant library. Position along the protein is shown on the x-axis and allele frequency is shown on they-axis. FIGS. 18D and 18E show the functionality of gene drive system against diverse genetic library in cis. PC9 cells expressing the EGFR variant library (red) were pooled without (FIG. 18D) and with (FIG. 18E) gene drive cells (green). Blue, orange, and purple arrows indicate osimertinib, dimerizer, and 5-FC treatment, respectively. The mean and standard error for three replicates are shown. FIG. 18F shows the schematic of genome-wide CRISPR library. The circular histogram depicts sgRNA abundance across the human genome. The final library is composed of 76,441 variants (and 1,000 non-targeting controls). FIG. 18G shows that PC9 cells were transduced with the lentiviral CRISPR library and pooled with GFP+ gene drive cells (S1vEGFRₒₛᵢ-S2vCyD; 5% spike-in). Pooled populations were treated and analyzed as in (FIG. 18B). FIG. 18H shows the volcano plot of hits in genome-wide CRISPR osimertinib screen. More resistant knockouts are shown in red.

FIG. 19A, 19B, 19C, 19D, 19E, 19F, 19G, and 19H show the diverse molecular designs can achieve evolutionary reprograming. FIG. 19A shows the schematic of modular dual-switch design. Alternative Switch 1 genes co-opting the kinase domains for various drug targets are shown. Additionally, orthogonal Switch 2 genes with demonstrable bystander activity are shown. FIG. 19B shows the schematic plasmid map of RET gene drive construct harboring Switch 1 (vRETprals) and Switch 2 (vCyD). FIGS. 19C and 19D show the functionality of RET gene drive in RET+ TPC1 cells. Sensitive (wild-type; blue) and resistant (CCDC6-RET G810R; red) cells were pooled without (FIG. 19C) and with (FIG. 19D) gene drive cells (S1vRETprals-S2vCyD; green). Pooled populations were treated with the RET inhibitor pralsetinib (blue arrow) and dimerizer (orange arrow) or 5-FC (purple arrow). Population dynamics for resistant cells are shown in the inset of (FIG. 19D). FIG. 19E shows the schematic plasmid map of immune gene drive construct harboring Switch 1 (vEGFRₒₛᵢ) and Switch 2 (vCD19). FIG. 19F shows the sensitive (wild-type; blue), resistant (C797S and mCherry+; red) and immune gene drive (S1vEGFRₒₛᵢ-S2vCD19 and GFP+; green) cells were pooled and treated with osimertinib and dimerizer. Upon outgrowth of the gene drive population, T cells and the CD19 bispecific T cell engager blinatumomab were added. FIGS. 19G and 19H show the functionality of immune gene drive in PC9 cells. Sensitive (blue) and resistant (red) cells were pooled without (FIG. 19G) and with (FIG. 19H) gene drive cells (S1vEGFRₒₛᵢ-S2vCD19; green). Blue, orange, and purple arrows indicate osimertinib, dimerizer, and T cells/blinatumomab, respectively.

FIG. 20A, 20B, 20C, 20D, 20E, 20F, and 20G show that the models inform optimal treatment regimens in vivo. FIG. 20A shows the theoretical treatment timelines for optimizing gene drives in vivo. Treatment scheduling may involve non-overlapping (top) or overlapping (bottom) Switch 1 and Switch 2 phases. FIG. 20B shows the results of stochastic dynamic model for optimizing switch scheduling in vivo. The model was parameterized using in vivo growth and drug kill rates. See Methods for more details. FIG. 20C shows the optimization of gene drive switch scheduling in PC9 tumors in vivo. Mixed populations of 50% resistant and 50% gene drive cells were grafted in mice to emulate a possible population structure at the beginning of the Switch 2 phase of treatment. Mice were treated once daily with osimertinib and 5-FC. The control group without switch overlap (purple) received no dimerizer. The “switch overlap” group (pink) received an initial two weeks of daily dimerizer treatment. FIG. 20D shows the sensitive (wild-type; blue), resistant (C797S and mCherry+; red) and gene drive (S1vEGFRₒₛᵢ-S2vCyD and GFP+; green) PC9 cells were pooled and grafted in mice. Upon tumor establishment, mice were treated with osimertinib (blue arrow) and dimerizer (orange arrow) or 5-FC (purple arrow). At various terminal time points, subsampled tumors were harvested, enzymatically digested, and analyzed by flow cytometry. FIG. 20E shows the functionality of optimized gene drive activity in vivo. Tumor volumes for populations of 0% gene drive (orange) and 10% gene drive (dark blue) are shown. Asterisks denote timepoints where subsampled tumors were analyzed by flow cytometry. These timepoints were at the beginning of Switch 1 treatment (DO; first asterisk), at the beginning of Switch 2 treatment (when tumors returned to their original volume; second asterisk), and 24 days after Switch 2 initiation or when the tumors exceeded 1.3× their original volume (whichever came first; third asterisk). The treatment schedule included a one week switch overlap. FIGS. 20F and 20G show the subpopulation analysis of tumors undergoing gene drive therapy. Population structure for 0% gene drive (FIG. 20F) and 10% gene drive (FIG. 20G) tumors are shown. Subpopulations are scaled to the relative tumor volumes at each timepoint. Timepoints correspond to asterisks in (FIG. 20D).

FIG. 21A, 21B, and 21C show the compartmental model outcomes as the “switch delay” parameter varies. FIG. 21A shows the switch delay parameter dictates the time between the beginning of Switch 2 treatment and the end of Switch 1 treatment, thus allowing for some overlap. While Switch 2 always begins when the gene drive population reaches the predetermined detection size, there may be benefit to maintaining Switch 1 for some period of time after Switch 2 initiation, as shown here. This is because Switch 1 activity is predicted to slow the collapse of the gene drive population, thus maximizing the Switch 2 bystander effect. However, if Switch 1 is maintained for too long, the gene drive population may serve as a reservoir for the development of cross-resistance.

FIG. 21B shows the sensitivity analysis of the compartmental model. For each parameter that is allowed to vary, probability of eradication (top heatmaps in each row) and median progression-free-survival (bottom heatmaps in each row) are shown. Parameter sweeps through net growth rate (top row) and turnover rate (second row) indicate that the model is relatively robust to growth kinetics. However, predicted outcomes worsen as tumor detection size (third row) and mutation rate (fourth row) increase. FIG. 21C shows the summary for linear regression analyses of eradication probability as predicted by gene drive cell dispersion. Results represent the p-value (γ-axis) of the cell dispersion metric (γ) in a model to predict eradication probability, for a range of bystander activity distances (p) along the x-axis. Gene drive dispersion is only significantly correlated with eradication probability for p=2.

FIGS. 22A, 22B, 22C, 22D, 22E, 22F, 22G, 22H, 22I, 22J, and 22K show the switch 1 activity in S1 vEGFRₑᵣₗ BaF3 cells in vitro. FIG. 22A shows the BaF3 cells expressing S1 vEGFR are expected to require dimerizer in the absence of IL-3. These cells were cultured in a range of dimerizer concentrations, and their growth rates were measured. The non-monotonic relationship between dimerizer dose and growth rate agrees with theoretical models of ligand-induced dimerization. FIG. 22B shows the switch 1 confers inducible resistance in S1 vEGFRen PC9 cells. EGFR+PC9 cells were transduced with the S1 vEGFRen gene and treated with a range of erlotinib concentrations in the presence (orange) or absence (gray) of dimerizer. Data for sensitive (wild-type; blue) and resistant (EGFR L858R/T790M; red) PC9 cells are also shown. FIG. 22C shows the schematic of Switch 1 vRETprals design. A G810R mutation in the RET kinase domain confers resistance to pralsetinib activity (left). An inducible FKBP12-RET fusion protein controllably induces RET signaling (right). A G810R resistance mutation in the RET kinase domain (S1 vRETprals) rescues signaling in the presence of pralsetinib. FIG. 22D shows the Switch 1 confers inducible resistance in S1 vRETprals TPC1 cells. RET+ TPC1 cells were transduced with S1 vRETprals and treated with a range of pralsetinib concentrations in the presence (orange) or absence (gray) of dimerizer. Data for sensitive (wild-type; blue) and resistant (CCDC6-RET G810R; red) TPC1 cells are also shown. FIGS. 22E, 22F, 22G, and 22H show the S2 vCyD activity across a panel of cancer cell lines. EGFR+PC9 (FIG. 22E), RET+ TPC1 (FIG. 22F), ALK+H3122 (FIG. 22G), and ROS1+ HCC78 (FIG. 22H) cells were transduced with S2 vCyD and treated with a range of 5-FC concentrations (light purple). Data for wild-type cells treated with 5-FC (blue) and 5-FU (the activated form of the drug; dark purple) are also shown. FIG. 22I shows the S2 vNfsA activity in 293T cells. The enzyme NfsA converts CB1954 into an active nitrogen mustard. Wild-type (blue) and S2 vNfsA (purple) 293T cells were treated with a range of CB1954 concentrations. FIG. 22J shows the switch 2 bystander activity for S2 vNfsA 293T cells treated with 100 μM CB1954, as in FIG. 9I. FIG. 22K shows the immune bystander activity in transwell plates. T cells (gold) were cocultured with CD19+ (purple) and CD19-(blue) PC9 cells with blinatumomab in the formats shown. After 48 hours, cells were stained and analyzed by flow cytometry to measure CD19-counts.

FIGS. 23A and 23B shows population and tumor growth dynamics. FIG. 23A shows the Population dynamics for in vitro experiment with gene drive cell spike-in (as in FIG. 14D) but without initial Switch 1 selection. Cells were treated with erlotinib and 5-FC from DO. FIG. 23B show the tumor growth dynamics for an in vivo experiment with gene drive cell spike-in (as in FIG. 14E) but without initial Switch 1 selection. Mice were treated with erlotinib and 5-FC from DO.

FIGS. 24A, 24B, 24C, 24D, and 24E show the switch 1 activity in complete gene drive S1vEGFRₒₛᵢ-S2vCyD PC9 cells. FIG. 24A shows that cells were treated with a range of osimertinib concentrations in the presence (orange) or absence (gray) of dimerizer. Data for sensitive (wild-type; blue) and resistant (EGFR L858R/C797S; red) PC9 cells are also shown. FIG. 24B shows the osimertinib-gene drive PC9 cells retain sensitivity to erlotinib. S1vEGFRₒₛᵢ-S2vCyD PC9 cells were treated with a range of erlotinib concentrations in the presence (orange) or absence (gray) of dimerizer. Data for sensitive (wild-type; blue), osimertinib-resistant (EGFR L858R/C797S; light red), and erlotinib-resistant (EGFR L858R/T790M; dark red) PC9 cells are also shown. FIG. 24C shows the switch 2 activity in complete gene drive S2vEGFRₒₛᵢ-S2vCyD PC9 cells. Wild-type (blue) and gene drive cells (purple) were treated with a range of 5-FC concentrations. FIG. 24D shows the bystander Switch 2 activity in complete gene drive S2vEGFRₒₛᵢ-S2vCyD PC9 cells. Mixtures of cells were pooled and treated with 1 mM 5-FC as in FIG. 9I. FIG. 24E shows the population dynamics for experiment with PC9 gene drive cell spike-in (as in FIG. 14D) but without initial Switch 1 selection. Cells were treated with osimertinib and 5-FC from DO.

FIGS. 25A, 25B, and 25C show the distribution of variant proportion in the EGFR variant library. FIG. 25A shows the values representing the proportion of each variant with respect to all other variants at that residue. FIG. 25B shows the replicate correlations for gene-level LFC values in genome-wide CRISPR screen for DMSO (left) and osimertinib (right) conditions. FIG. 25C shows the separation of essential and nonessential/non-targeting control guides for replicates of the DMSO condition. NNMD quality control metric from53 is shown. NNMD values below a threshold of −1 are generally thought to exhibit good separation between controls.

FIG. 26 shows the population dynamics for experiment with RET gene drive cell spike-in (as in FIG. 19D) but without initial Switch 1 selection. Cells were treated with pralsetinib and 5-FC from DO.

FIG. 27A, 27B, 27C, 27D, 27E, 27F, and 27G show the tumor dynamics for a range of initial gene drive frequencies under Switch 2 treatment. FIG. 27A shows the mixed populations of PC9 cells reflecting potential population structures at the end of Switch 1 treatment were grafted in mice. Mice were treated once daily with osimertinib and 5-FC. FIG. 27B shows the tumor dynamics for the complete gene drive system in PC9 cells, as in FIG. 20E. Tumor volumes for 0% gene drive (dark orange) and 0.3% gene drive (light orange) are shown. Asterisks indicate timepoints for tumor harvesting and analysis. The second asterisk also indicates the initiation of Switch 2 treatment. FIG. 27C shows the subpopulation analysis of tumors undergoing gene drive therapy (as in FIGS. 20F and 20G) for 0.3% gene drive populations. FIG. 27 D shows the tumor dynamics as in FIG. 27B for 1% gene drive populations. FIG. 27E shows the subpopulation analysis as in FIG. 27C for 1% gene drive populations. FIG. 27F shows the tumor dynamics as in FIG. 27B for 3% gene drive populations. FIG. 27G shows the subpopulation analysis as in FIG. 27C for 3% gene drive populations.

### DETAILED DESCRIPTION

The following description of the disclosure is provided as an enabling teaching of the disclosure in its best, currently known embodiment. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Definitions

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10″ as well as “greater than or equal to 10” is also disclosed. It is also understood that the throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

By “reduce” or other forms of the word, such as “reducing” or “reduction,” means lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The terms “treat,” “treating,” “treatment,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially. Treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

A “protein”, “polypeptide”, or “peptide” each refer to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like.

A “promoter,” as used herein, refers to a sequence in DNA that mediates the initiation of transcription by an RNApolymerase. Transcriptional promoters may comprise one ormore of a number of different sequence elements as follows: 1) sequence elements present at the site of transcription initiation; 2) sequence elements present upstream of the transcription initiation site and; 3) sequence elements down-stream of the transcription initiation site. The individual sequence elements function as sites on the DNA, where RNA polymerases and transcription factors facilitate positioning of RNA polymerases on the DNA bind.

As used herein, “downstream” refers to the relative position of a genetic sequence, either DNA or RNA. Downstream relates to the 5′ to 3′ direction relative the start site of transcription, wherein downstream is usually closer to the 3′ end of a genetic sequence.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrametric glycoproteins of about 150,000 Daltons, composed of two identical light (L) chains and two identical heavy (H) chains. Each heavy chain has at one end a variable domain (VH) followed by a number of constant domains. Each light chain has a variable domain at one end (VL) and a constant domain at its other end.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

A “gene” refers to a polynucleotide containing at least one open reading frame that is capable of encoding a particular polypeptide or protein after being transcribed and translated. Any of the polynucleotides sequences described herein may be used to identify larger fragments or full-length coding sequences of the gene with which they are associated. A resistance gene as used herein refers to a gene that encodes a drug resistant peptide, polypeptide, protein, or receptor. A suicide gene as used herein refers to a gene that encodes an enzyme that metabolizes or converts an administered prodrug into an active drug, which targets and kills cancer cells.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, “operably linked” refers to two or more genes, peptides, polypeptides, proteins, compositions, compounds, or molecules being bound or linked together in such a way the optimizes the intended function. When bound or linked, these genes, peptides, polypeptides, proteins, compositions, compounds, or molecules can be linked covalently, electrostatic interaction, through hydrogen bonding, or any combinations thereof.

As used herein, a “prodrug” refers to a compound or composition that after administration or ingestion is metabolized into a pharmaceutically active drug. Prodrugs can also be viewed as compounds or compositions containing specialized nontoxic protective properties used in a transient manner to alter or eliminate undesirable properties of the active drug.

A “nucleic acid” is a chemical compound that serves as the primary information-carrying molecules in cells and make up the cellular genetic material. Nucleic acids comprise nucleotides, which are the monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

A “receptor is a cellular protein whose activation causes a cell to modify its present functions or actions.

A “fitness benefit compound” refers to a compound, molecule, biomolecule (such as, for example, a nucleotide, nucleic acid, amino acid, peptide, polypeptide, protein, lipid, or carbohydrate), or supplement used to promote cell growth, proliferation, and/or differentiation. The fitness benefit compound can be encoded by a nucleic acid composition. In some embodiments, the fitness benefit compound can be administered in combination with any other component or feature of a gene selection drive system. Conversely, in some embodiments, growth, proliferation, differentiation can also be promoted in the absence of one or more fitness benefit compounds including, but not limited to a molecule, biomolecule (such as, for example, a nucleotide, nucleic acid, amino acid, peptide, polypeptide, protein, lipid, or carbohydrate), or supplement.

A “fitness cost gene” refers to a nucleic acid sequence that encodes a protein, polypeptide, or peptide causing lethality to a cell or tissue. As used herein, the fitness cost gene comprises a “Bystander Effect” to target the remaining 1% or more of cells remaining after cell death caused by a suicide enzyme, protein, polypeptide, or peptide. As used herein, the “Bystander Effect” refers to a biological response or an activation of a gene resulting from an original event, such as cell death, from an adjacent or nearby cell. In some embodiments, the original event is cell death due to a suicide enzyme to kill a large portion or percentage of cells. Then, the “fitness cost gene” activates to kill a smaller portion or percentage of remaining cells due to the “Bystander Effect”. Such events depend on intercellular communications and amplify the actions and/or consequences of the original event.

### Nucleic Acid Compositions

In one aspect, disclosed herein are nucleic acid compositions comprising a fitness benefit compound, a fitness cost gene (such as, for example, a cytosine deaminase gene, a NADPH nitroreductase gene, or a diptheria toxin gene), and a promoter within a pharmaceutically acceptable carrier, wherein the resistance gene comprises a dimerization domain gene operably linked to a drug resistant gene. In some aspects, the dimerization protein (such as for example, FK506-binding protein 12 (FKBP12)) is fused to the drug resistance receptor.

In some embodiments, the fitness benefit molecule comprises a resistance gene, metabolite, a growth factor, a cytokine, a supplement, or a biomolecule thereof. In some embodiments, the fitness cost gene is a suicide gene.

As used herein, “drug resistant genes” refer to any aberrantly expressed gene or gene mutation that confers resistance to an anti-cancer therapeutic. In some aspect, the resistance gene can be a mutated receptor tyrosine kinase gene including, but not limited to epidermal growth factor receptor, including HER-2, HER-3, HER-4, epidermal growth factor receptor (EGFR), Vascular Endothelial Growth Factor Receptor (VEGFR), platelet-Derived Growth Factor Receptor (PDGER), and Fibroblast Growth Receptor (FGR), anaplastic lymphoma kinase (ALK), ROS1, RET, or MET.

There are many suicide genes that are known in the art and can be used in the disclosed nucleic acid compositions. Examples of such genes include a cytosine deaminase genes (including, but not limited to cytosine deaminase-5-fluorocytosine), NADPH nitroreductase genes (including, but not limited to nitroreductase-5-[aziridin-1-yl]-2,4-dinitrobenzamide), herpesvirus thymidine kinase (HSV/Tk) gene, cytochrome P450-ifosfamide, cytochrome P450-cyclophosphamide, or diptheria toxin genes.

It is understood and herein contemplated that the relative order of the fitness benefit gene and the fitness cost gene in the nucleic acid can be relevant. In some aspects, the fitness cost gene is located downstream of the resistance gene. Additionally, the size of the fitness benefit gene and the fitness cost gene can be important for function and delivery to the cell. In some embodiments, the fitness benefit gene is 2 or more kilobases in length. In some embodiments, the fitness benefit gene is 2, 3, 4, 5, 6, 7, 8, 9, 10, or more kilobases in length. In some embodiments, the fitness cost gene is at least 0.25, 0.5, 0.75, 1.0, 1.1, 1.2, 1.3, 1.4, 1.5, 1.6, 1.7, 1.8, 1.9, 2.0, 2.1, 2.2, 2.3, 2.4, or 2.5 kilobases (kb) in length. Also disclosed herein are nucleic acid compositions, wherein the fitness cost gene is at least 0.25 kilobases (kb) in length.

In some embodiments, the dimerization domain gene encodes a dimerizing protein. In some embodiments, the drug resistant gene encodes a drug resistant receptor. In some embodiments, the dimerizing protein is fused to the drug resistant receptor. In some embodiments, the drug resistant receptor is a drug resistant tyrosine kinase receptor.

In some embodiments, the fitness benefit molecule comprises a metabolite, a growth factor, a cytokine, a supplement, or a biomolecules thereof.

In some embodiments, the fitness cost gene encodes a suicide enzyme. In some embodiments, the fitness cost gene is a cytosine deaminase gene, a NADPH nitroreductase gene, or a diptheria toxin gene.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier. In some embodiments, the engineered resistance gene, or the fitness benefit compound thereof, suicide gene, or the fitness cost gene thereof, and promoter are encoded on a retroviral vector including, but not limited to a lentiviral vector.

In one aspect, disclosed herein are cells comprising the nucleic acid compositions of any preceding aspect.

### Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as are nucleic acid compositions comprising a fitness benefit gene, a fitness cost gene (such as, for example, a cytosine deaminase gene, a NADPH nitroreductase gene, or a diptheria toxin gene), and a promoter into the cell without degradation and include a promoter yielding expression of the gene in the cells into which it is delivered. In some embodiments the vectors delivering the nucleic acid to a cell are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsulation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### Retroviral Vectors

A retrovirus is an animal virus belonging to the virus family of Retroviridae, including any types, subfamilies, genus, or tropisms. Retroviral vectors, in general, are described by Verma, I.M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically, a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals. In one aspect disclosed herein are nucleic acid compositions, wherein the engineered resistance gene, suicide gene, and promoter are encoded on a retroviral vector including, but not limited to a lentiviral vector.

### Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically, the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors and may contain upstream elements and response elements.

### Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8:33-41, 1994; Cotter and Robertson, Curr Opin Mol Ther 5:633-644, 1999). These large DNA viruses (herpes simplex virus (HSV) and Epstein-Barr virus (EBV), have the potential to deliver fragments of human heterologous DNA>150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

### Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors and may contain upstream elements and response elements.

### Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g., beta actin promoter. The early and late promoters of the SV40 virus are conveniently obtained as an SV40 restriction fragment which also contains the SV40 viral origin of replication (Fiers et al., Nature, 273:113 (1978)). The immediate early promoter of the human cytomegalovirus is conveniently obtained as a HindIII E restriction fragment (Greenway, P. J. et al., Gene 18:355-360 (1982)). Of course, promoters from the host cell or related species also are useful herein. In addition, tissue specific promoters (including, but not limited to surfactant protein B promoter (SP-B in lung), B29 promoter (B cells), CD14 promotor (monocytic cells), CD43 promoter (leukocytes and platelets), CD68 promoter (macrophages), Desmin promoter (muscle), Elastase-1 promoter (pancreatic acinar cells), endoglin promoter (endothelial cells), Fibronectin promoter (differentiating cells and healing tissues), Flt-1 promoter (endothelial cells), GFAP promoter (astrocytes), Mb promoter (muscle), SYN1 promoter (neurons), SV40/bAlb promoter (Liver)) and cancer specific promoters (including, but not limited to carcinoembryonic antigen (CEA) promoter, hTERT promoter, epidermal growth factor receptor (EGFR) promoter, human epidermal growth factor receptor/neu (HER2/NEU) promoter, vascular endothelial growth factor receptor (VEGFR) promoter, folate receptor (FR) promoter, transferrin receptor (CD71) promoter, mucines promoters, tumor resistance antigen 1-60 (TRA-1-60) promoter, cyclooxygenase (COX) promoter, cytokeratin 18 promoter, cytokeratin 19 promoter, surviving promoter, and chimeric antigen receptor (CAR) promoters, alpha-fetoprotein (AFP) promoter, thyroid transcription factor 1 (TTF-1) promoter, glypican-3 protein (GPC3) promoter, human secretory leukocyte protease inhibitor (hSLPI) promoter, ERBB2 promoter, Mucin 1 (MUC1) promoter, L-plastin promoter, alpha-lactalbumin (LALBA) promoter, cyclooxygenase 2 (COX2) promoter, epithelial glycoprotein (EPG2) promoter, A33 promoter, uPAR promoter, breast cancer 1 (BRCA1) and BRCA2 promoters) are useful herein.

Enhancer generally refers to a sequence of DNA that functions at no fixed distance from the transcription start site and can be either 5′ (Laimins, L. et al., Proc. Natl. Acad. Sci. 78:993 (1981)) or 3′ (Lusky, M. L., et al., Mol. Cell Bio. 3:1108 (1983)) to the transcription unit. Furthermore, enhancers can be within an intron (Banerji, J. L. et al., Cell 33:729 (1983)) as well as within the coding sequence itself (Osborne, T. F., et al., Mol. Cell Bio. 4:1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin,-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs. In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

It is understood and herein contemplated that the disclosed nucleic acid compositions obtain their functionality when encoded in a cell. Accordingly, also disclosed herein are cells comprising the nucleic acid compositions disclosed herein.

### Gene Drive Systems

In one aspect, disclosed herein are gene selection drive systems comprising the nucleic acid composition of any preceding aspect, wherein said system is activated in a cell population comprising a dimerizer (such as, for example, a peptide, polypeptide, or small molecule including, but not limited to FK506-binding protein 12 (FKBP12) peptide or a dihydrofolate reductase (DHFR) polypeptide) and a therapeutic compound (such as, for example, an anti-cancer therapeutic including, but not limited to prodrugs of anti-cancer therapeutics). For example, disclosed herein are gene selection drive systems comprising a fitness benefit molecule, a fitness cost gene (such as, for example, a cytosine deaminase gene, a NADPH nitroreductase gene, or a diptheria toxin gene), and a promoter within a pharmaceutically acceptable carrier, wherein the fitness benefit molecule comprises a dimerization domain gene operably linked to a drug resistant gene. In some aspects, the dimerization protein is fused to the drug resistance receptor. In one aspect, the nucleic acid can be encoded in a cell, including, but not limited to a cell population (such as, for example, a cell population comprising a first, second, and/or third cell).

In some embodiments, the dimerizer and the therapeutic compound are administered simultaneously or individually to the cell population.

In some embodiments, the dimerizer is a peptide, a polypeptide, or a small molecule. In some embodiments, the dimerizer is a FK506-binding protein12 (FKBP12) peptide. In some embodiments, the active drug is a chemotherapy drug. In some embodiments, the dimerizer is an F36V mutant of the FKBP12 peptide. In some embodiments, an AP20187 ligand is used to induce dimerization of the F36V mutant.

Also disclosed herein are gene selection drive systems, wherein the dimerizer interacts with one or more dimerizing proteins fused to the drug resistant receptor to induce drug resistance in the first cell, wherein the therapeutic compound kills the second cell, and wherein the third cell comprises an innate drug resistance.

In some embodiments, the suicide enzyme is expressed in the first and the third cell. In some aspects, the suicide enzyme is expressed in response to a physical stimulus (such as for example, increased population of cells), chemical stimulus (such as, for example, a doxycycline compound or a tetracycline compound), or a genetic stimulus (such as for example, a tissue specific promoter or a tumor specific promoter).

In addition, tissue specific promoters (including, but not limited to surfactant protein B promoter (SP-B in lung), B29 promoter (B cells), CD14 promotor (monocytic cells), CD43 promoter (leukocytes and platelets), CD68 promoter (macrophages), Desmin promoter (muscle), Elastase-1 promoter (pancreatic acinar cells), endoglin promoter (endothelial cells), Fibronectin promoter (differentiating cells and healing tissues), Flt-1 promoter (endothelial cells), GFAP promoter (astrocytes), Mb promoter (muscle), SYN1 promoter (neurons), SV40/bAlb promoter (Liver)) and cancer specific promoters (including, but not limited to carcinoembryonic antigen (CEA) promoter, hTERT promoter, epidermal growth factor receptor (EGFR) promoter, human epidermal growth factor receptor/neu (HER2/NEU) promoter, vascular endothelial growth factor receptor (VEGFR) promoter, folate receptor (FR) promoter, transferrin receptor (CD71) promoter, mucines promoters, tumor resistance antigen 1-60 (TRA-1-60) promoter, cyclooxygenase (COX) promoter, cytokeratin 18 promoter, cytokeratin 19 promoter, surviving promoter, and chimeric antigen receptor (CAR) promoters, alpha-fetoprotein (AFP) promoter, thyroid transcription factor 1 (TTF-1) promoter, glypican-3 protein (GPC3) promoter, human secretory leukocyte protease inhibitor (hSLPI) promoter, ERBB2 promoter, Mucin 1 (MUC1) promoter, L-plastin promoter, alpha-lactalbumin (LALBA) promoter, cyclooxygenase 2 (COX2) promoter, epithelial glycoprotein (EPG2) promoter, A33 promoter, uPAR promoter, breast cancer 1 (BRCA1) and BRCA2 promoters) are useful herein.

In one aspect, disclosed herein are gene selection drive systems, wherein the suicide enzyme converts a prodrug into an active drug. In some aspects, the active drug kills the first cell and third cell or a residual cell not comprising the system.

In one aspect, disclosed herein are cells comprising the gene selection drive system or nucleic acid composition of any preceding aspect.

### Methods of Treating Cancer

The disclosed compositions can be used to treat, inhibit, decrease, reduce, ameliorate and/or prevent any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

In one aspect, the treatment of the cancer can include the administration of the gene selection drive system or any of the disclosed nucleic acid compositions to a subject in need thereof. For example, methods of treating, inhibiting, decreasing, reducing, ameliorating, and/or preventing a cancer and/or metastasis in a subject in need thereof, the method comprising administering to the subject the gene selection drive system or nucleic acid composition disclosed herein. For example, disclosed herein are methods of treating, inhibiting, decreasing, reducing, ameliorating, and/or preventing a cancer and/or metastasis in a subject in need thereof, the method comprising administering to the subject a gene selection drive system or a nucleic acid composition comprising an engineered resistance gene, a suicide gene (such as, for example, a cytosine deaminase gene, a NADPH nitroreductase gene, or a diptheria toxin gene), and a promoter within a pharmaceutically acceptable carrier, wherein the resistance gene comprises a dimerization domain operably linked to a drug resistant target gene. In some aspects, the one or more dimerizing domain is fused to a drug resistant receptor to induce drug resistance in the tumor.

In one aspect, disclosed herein are methods of treating, inhibiting, decreasing, reducing, ameliorating, and/or preventing a cancer and/or metastasis in a subject in need thereof, the method comprising administering to the subject the gene selection drive system or nucleic acid composition of any preceding aspect. For example, disclosed herein are methods of treating, inhibiting, decreasing, reducing, ameliorating, and/or preventing a cancer and/or metastasis in a subject in need thereof, the method comprising administering to the subject a gene selection drive system or a nucleic acid composition comprising a fitness benefit molecule, a fitness cost gene (such as, for example, a cytosine deaminase gene, a NADPH nitroreductase gene, or a diptheria toxin gene), and a promoter within a pharmaceutically acceptable carrier, wherein the resistance gene comprises a dimerization domain operably linked to a drug resistant target gene. In some embodiments, the system is activated in a tumor of the subject when a dimerizer and a therapeutic compound are further administered simultaneously or individually.

In some embodiments, the dimerizer is a peptide, polypeptide, or small molecule. In some embodiments, the dimerizer is a FK506-binding protein 12 (FKBP12) peptide.

In some embodiments, the one or more dimerizing domain is fused to a drug resistant receptor to induce drug resistance in the tumor.

In some embodiments, the fitness benefit molecule promotes cell growth in the subject. In some embodiments, the fitness cost gene encodes a suicide enzyme whereby said suicide enzyme converts a prodrug into an active chemotherapeutic drug. In some embodiments, the fitness cost gene is a cytosine deaminase gene, a NADPH nitroreductase gene, or a diptheria toxin gene.

In some aspects, the therapeutic compound and the active chemotherapeutic drug kill at least 80% of cancer cells in the tumor. In some embodiments, the fitness cost gene kills the remaining 1-20% of cancer cells in the tumor.

It is understood and herein contemplated that anti-cancer therapeutic used in the disclosed methods, nucleic acid compositions, and gene selection drive systems disclosed herein can be any anti-cancer therapeutic known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U(Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU(Fluorouracil Injection), 5-FU(Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS(Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS(Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep)

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: Forward Engineering of a “Dual Switch” Drive in Non-Small Cell Lung Cancer

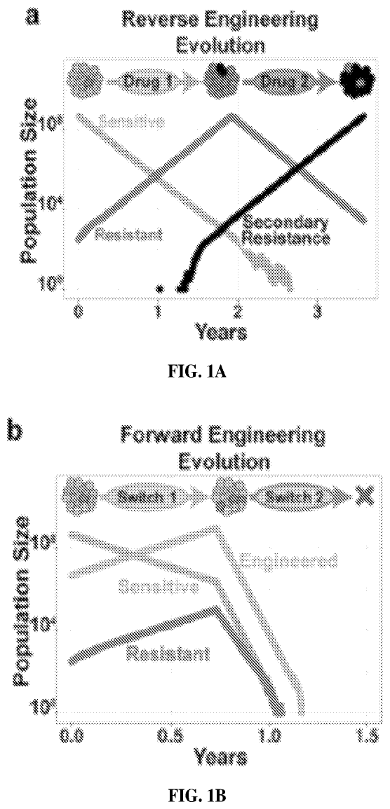

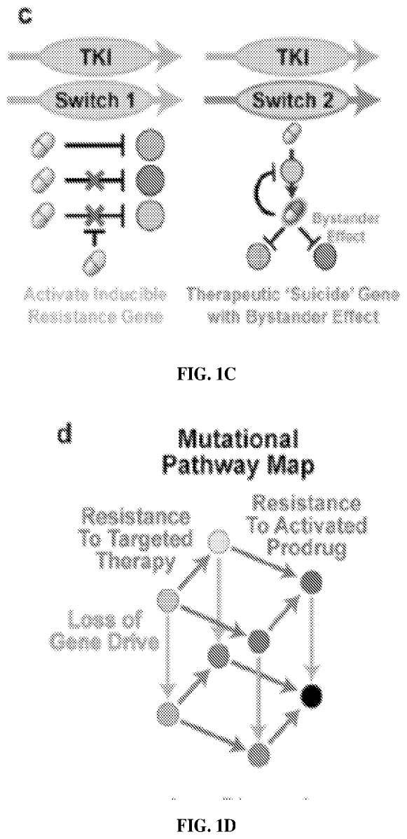

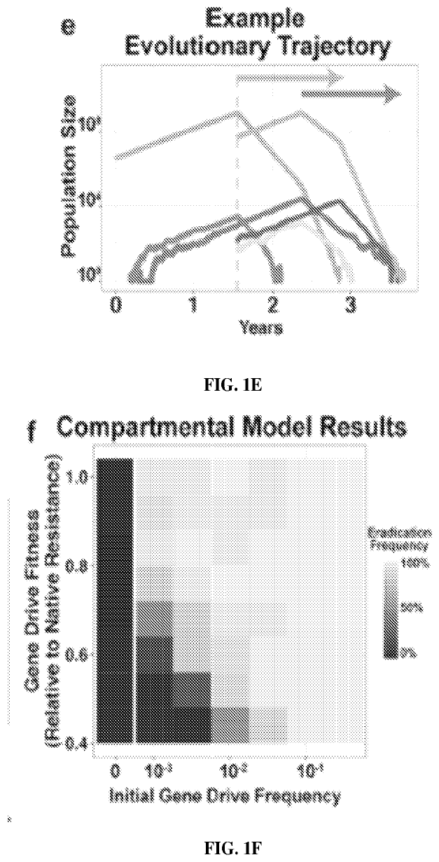

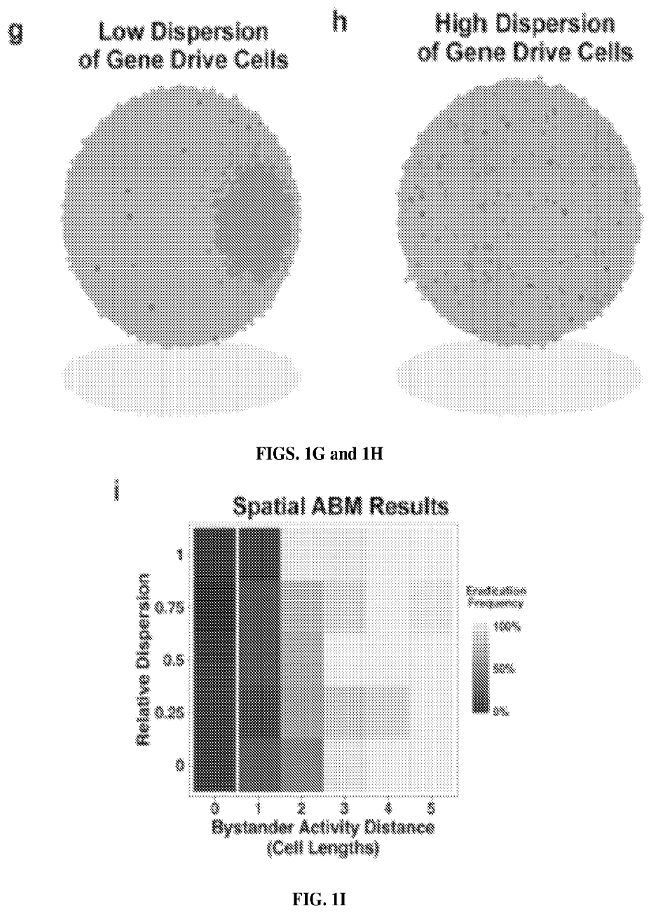

In Non-small-cell-lung cancer (NSCLC), specific mutations in EGFR, RET, ALK, ROS1, MET and TRK give rise to driver oncogenes that result in tumors with potent clinical responses to tyrosine kinase inhibitors. But decades of traditional drug development have locked oncologists into an evolutionary arms race between drug discovery and drug resistance (FIG. 1). Advances in single cell “omic” techniques enabled high resolution measurements of single cell evolutionary heterogeneity during drug treatment. However, these higher resolution measurements have revealed that resistance mutations often pre-exist, and new drugs simply lead to new cycles of resistance (FIG. 1A). This resistance has a diversity of molecular mechanisms and few options. Thus, “reverse engineering” drug resistance after a clinical failure guarantees that resistance wins. (FIG. 1A). A second approach, gene directed prodrug therapy, introduces suicide genes into cancer cells to create localized killing without systemic toxicity. But suicide gene therapy is typically limited by efficiency. A forward engineering design is used to create synthetic biology solutions that fight drug resistance evolution in tyrosine kinase-driven cancers and compensate for the efficiency issues in early suicide gene approaches (FIG. 1A). This is done by connecting a “selection gene drive” to a suicide gene that kills resistant cells, independent of molecular mechanism of drug resistance.

A dual-switch gene drive approach is presented that utilizes selection. Switch 1 uses the clinical grade chemically induced dimerization domain, FKBP12 F36V, coupled to drug resistant versions of tyrosine kinases to temporarily outcompete pre-existing resistance clones, independent of molecular resistance mechanisms. Switch 2 is a suicide gene that hitchhikes on switch 1 and creates cell killing toxins in the tumor microenvironment. These toxins have a “bystander effect” whereby local diffusion in the microenvironment can kill all cells, including those that are never infected with a gene drive. A selection drive works with standard-of-care kinase inhibitors to maximize the bystander effect and independently kill pre-existing resistance mutations (FIG. 1B).

Dual-switch gene drives are optimized for NSCLC. Modeling has identified design principles for optimal Switch 1+2 function. Different cancer cell lines have been shown to exhibit different sensitivities to the killing effect of switch 2. Validation experiments coupled to failure models have shown new design goals for both switch 1 and 2. These designs highlight improved performances across diverse patients. This example builds pharmacologically tuned switch 1 designs and examines the personalization of specific switch 2 prototypes. Beyond personalization, model-driven insights that show inducible resistance to switch 2 constructs and triple-switch drives is tested.

Dual-switch gene drives are investigated in heterogenous microenvironments. Gene drive prototypes have been developed, but they fail via “evolutionary risk”. These risks are spatial, mutational, microenvironmental (extracellular matrix/stroma) and pharmacologic. 3D agent-based models have been developed that incorporate mutational and spatial risk in the presence of diverse microenvironmental cues. Competition between gene drives, diverse tumor clones, matrix interactions, cancer associated fibroblasts, immune and endothelial cells are tested. Toxicity in non-cancerous cells and control thereof is also tested.

Prototypes in heterogeneous patient derived organoids are tested. Existing prototypes are tested in organoids grown in lung mimetic microenvironments. Organoids are infected with switches 1 and 2 and tumor evolution and therapeutic responses are directly measured. A genetic library of evolutionary mechanisms are introduced to assess the efficacy of dual-switch designs faced with diverse resistance mechanisms.

Construction of dual-switch selection drives allows for preventing drug resistance.

### Example 2: Intracellular and Extracellular Interactions of “Dual Switch” Selection Drives

A way to get in front of drug resistance and outpace evolution is to use combination therapy. Combinations can be clinically curative in childhood leukemias and ˜50% of adult diffuse large B-cell lymphomas. Here, even standard chemotherapy can have a tremendous therapeutic window to kill cancer cells with tolerable toxicity in normal cells. However, identifying a combination for NSCLC where multiple drugs exhibit targeted-therapy-like clinical safety and efficacy is challenging. Combinations have been tested in EGFR and ALK positive NSCLC patients based on clinical hypotheses and mechanism-driven preclinical investigations. However, none of these combinations have become broadly accepted as practice changing standards of care because the increases in overall survival in patients are modest, mixed, or insignificant. Beyond mutations in the drug target, mutations outside the drug target are common. This is exemplified in the preclinical identification of MET, Yes1, and IGFIR as potential tyrosine kinase resistance mechanisms and it has led to the proposition that inhibitors of these proteins should be tried in combination with EGFR or ALK tyrosine kinase inhibitors (TKIs). However, the percentage of total patients with any one of these individual off-target resistance mechanisms is small. Thus, to make a clinical impact, patients that develop a specific resistance mechanism must be pre-identified. This is because the combination only inhibits a minor clone in a small percentage of patients, and therefore does not achieve the benefits of 2 independent agents that both have high log kills. Consistent with this, clinical evidence shows that MET, Yes1, or IGFIR combinations with EGFR inhibition does not improve response rates or progression free survival. Thus, empirical and mechanistic approaches to combination therapy in patients with targeted therapy responsive NSCLC have failed to generate dramatic additional benefits.

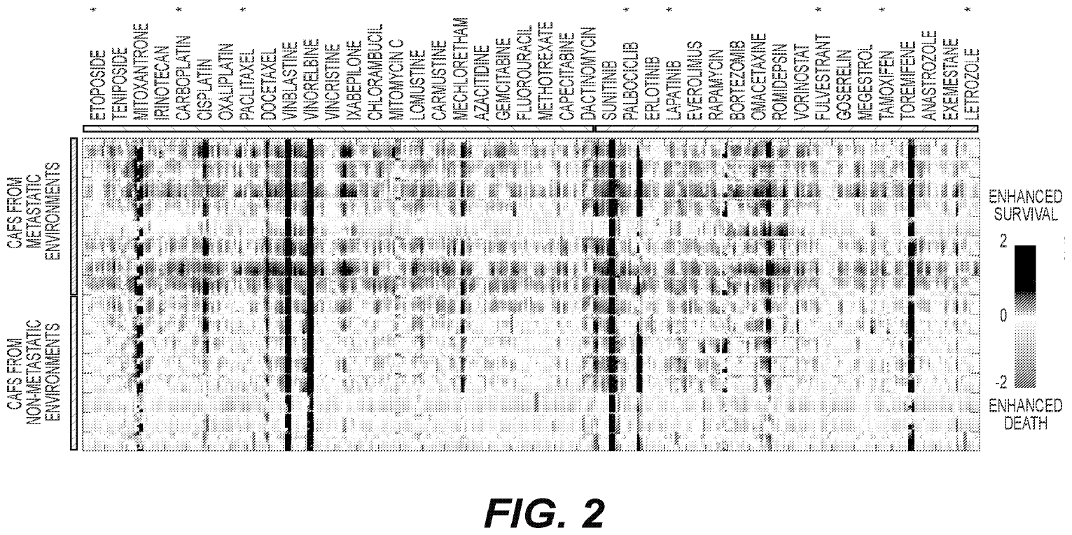

Beyond cell intrinsic mechanisms, targeted therapy has widely displayed the tumor stroma and the extracellular matrix composition as impactful on resistance to tyrosine kinase inhibitors. Although, it is unclear how the microenvironment would impact the growth and drug responsiveness of dual-switch selection drives in cancer. The ability of gene-drive driven cell therapies to grow and respond to drugs in different locations due to both intrinsic properties of cells (i.e., driven by mutations), and driven by interactions between cancer cells and the microenvironment has been shown (FIG. 2). Using synthetic environments created to mimic microenvironmental niches, in combination with computational modeling to de-convolve the relative influences of both the genetic and environmental features of tumors, how both these intra- and extra-cellular factors interact is determined using dual-switch selection drives.

### Example 3: The Forward Engineering Design of Tumor Evolution to Develop Targeted Cancer Therapy

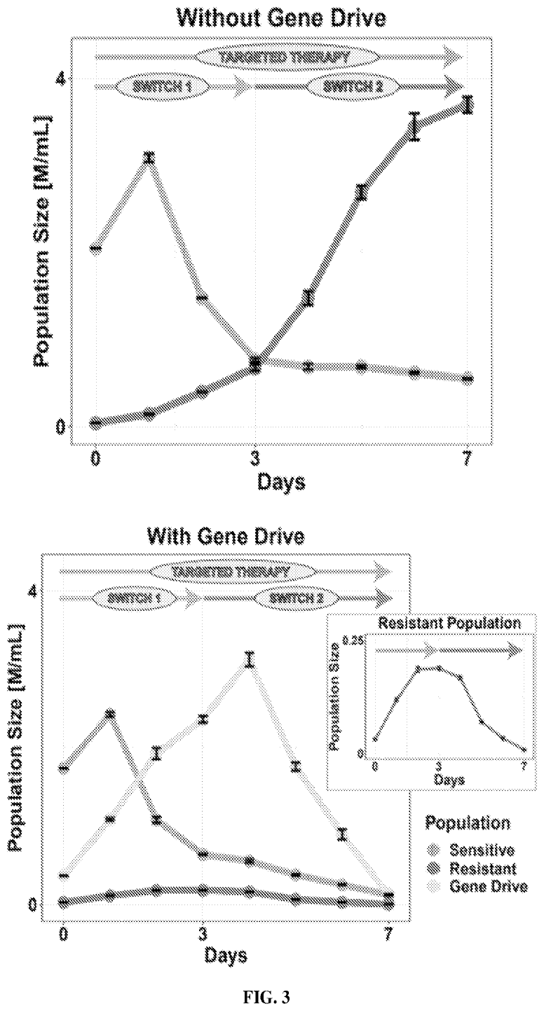

Synthetic biologists have recently proposed using CRISPR-Cas9 gene drive technologies to control disease vector evolution. These CRISPR-Cas9 gene drives use gene editing to cut the second allele of a gene in a diploid organism. This strongly biases allele transmission during sexual reproduction. However, microbes, immune cell therapies, and cancers represent asexual challenges to a gene-drive-like approach that requires completely different designs. Furthermore, in large asexual populations, genetic heterogeneity is the rule. Thus, any attempt to therapeutically evaluate a gene drive approach in a large population (such as during drug treatment) is thwarted by pre-occurring evolutionary diversity. This example controls unicellular asexual populations using “dual-switch selection gene drives”. Relying on selection instead of inheritance allows for focus on a different fundamental evolutionary force to succeed where Cas9 gene drives fail. FIG. 3 is a demonstration of a dual-switch selection drive to design the evolutionary population dynamics of an EGFR mutation driven model of cancer.

A synthetic biology therapy works with an existing clinical standard of care in NSCLC. While current switch 2 designs are delivered via a tumor homing bacteria or virus these delivery vehicles do not always work with existing standards of care. For instance, tyrosine kinase inhibitors have been shown to inhibit bacterial and viral replication or motility in cells and animals. This is a problem for tyrosine kinase driven NSCLCs that are treated with kinase inhibitors. Most patients have large and durable objective responses to tyrosine kinase treatment. Moreover, previous iterations of suicide gene therapy in the clinic have failed because of delivery efficiency. The evolutionary pressures that are known to occur during tyrosine kinase treatment are utilized to maximize suicide gene delivery, instead of attempting to replace an established therapeutic agent with a synthetic organism.

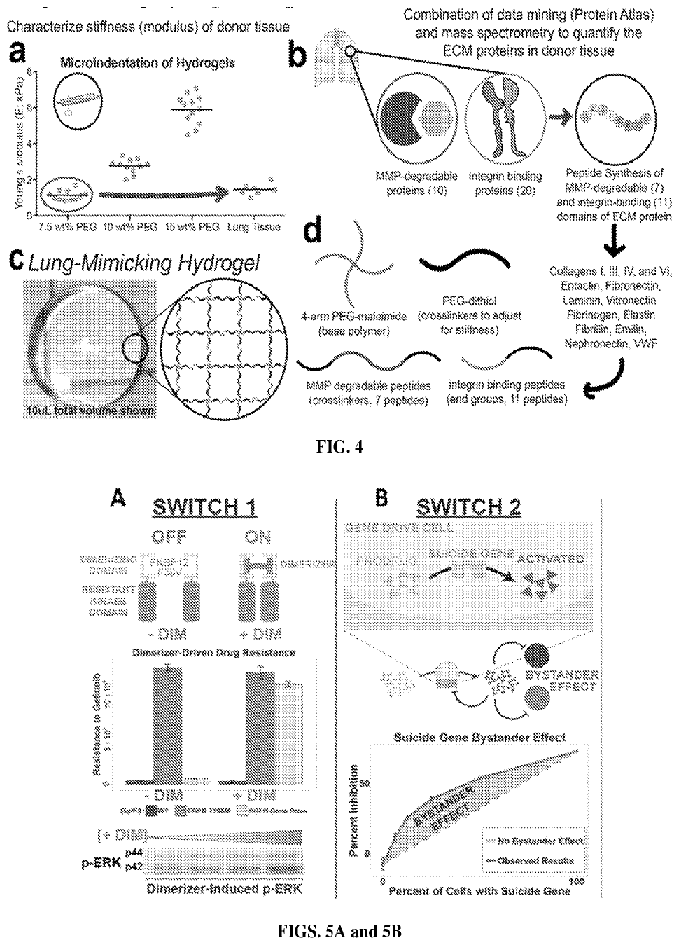

A heterogeneous bioengineered system is used to guide tumor wide analyses of the changes in population dynamics driven by a synthetic biology technology. Tumor biology is complex, and many different cell types inhabit a tumor bed. This heterogeneous group of cells, and the ECM these cells create, form a complex ecosystem that evolves during tumor treatment and can enhance or inhibit responses to therapy. To fully test NSCLC synthetic gene drive approach in more realistic in vitro settings, a cell culture system that is fully controllable is used. The system is more akin to real lung tissue than tissue culture plastic but maintains compatibility with high-throughput methods that facilitate failure testing across many conditions. The field now appreciates that tissue culture polystyrene (TCPS) plates, the plastic surfaces upon which drugs are developed by industry world-wide, fail to represent the complex cell-ECM interactions of real tissue, and omission of ECM from in vitro testing is partially responsible for failure to test new effective drugs. With tunable, more realistic environments, cell-ECM interactions are perturbed systematically and can easily screen across large swaths of conditions, drugs, stromal cell compositions and time points. Combined with systems-level analyses and modeling, these readouts are translated to in vivo studies in a predicted, heavily narrowed fashion. Designs of synthetic ECMs, made from simple poly(ethylene glycol) (PEG) and peptides, to represent key biochemical (integrin binding, proteolytic degradability) and biomechanical (stiffness) features of tissue were designed. These PEG-based gels rapidly polymerize around cells at physiological temperatures, salt concentrations, and pH, making them ideal for 3D culture. There are key distinctions between this 3D cell culture platform and others that have been previously used. The bulk of PEG-based biomaterials used with cells are biochemically limited: typically including a single αᵥβ₃ binding domain (the RGD amino acid sequence) to facilitate cell adhesion. Taking a tissue-specific approach a method was developed which combines literature mining from the Protein Atlas and quantitative mass spectrometry (LC/MS-MS), to identify the cell adhesive (integrin-binding) and proteolytically degradable (MMP) ECM proteins within the lung (FIG. 4). The lung hydrogels will contain the peptide domains responsible for integrin binding and MMP degradation from lung ECM proteins found consistently on the protein and RNA level across multiple patients in the Protein Atlas (10 peptides for integrin-binding domains; and 7 peptides for protease cleavage domains (FIG. 4B, 4C, and 4D)). Peptides must be used in lieu of full ECM proteins to avoid disturbing the gel network (mesh size constraints). These peptides are crosslinked into the gel as drawn in FIG. 4c during cell encapsulation. In addition to biochemical signaling, integrin-ECM interactions impart mechanical cues via strengthening of cell adhesion and intracellular tension. It is therefore imperative to include the direct effects of ECM biomechanical stimuli, which varies across tissues. Lung, like PEG, is a highly elastic material, and by tuning the crosslinker density, the synthetic gel has the same modulus as lung parenchyma (FIG. 4A). This tunable lung ECM microenvironment is used to test the fidelity of synthetic biology gene drive approach to be efficacious across diverse environments, independent of drug resistance mechanism.

### Example 4: Modeling Drug Resistance Evolution to Improve Drug Design

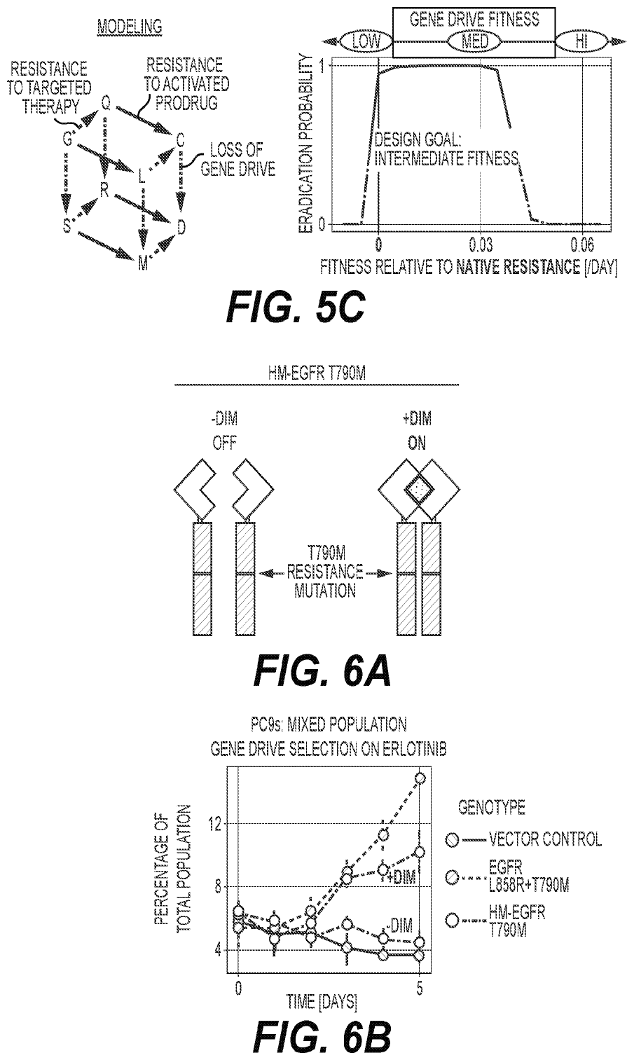

A selection gene drive was designed (Switch 1, FIG. 5A) that is a distinct and inducible drug resistant version of a drug target gene. It works by selection during targeted therapy of the original gene. A “dual-switch selection drive” attaches a suicide gene (Switch 2, FIG. 5B) (with a bystander effect) to the selection drive. This suicide gene hitchhikes on the selection drive until the prodrug is administered. Hitchhiking means that most of the population will contain the gene drive, and therefore, the suicide gene. Hitchhiking maximizes the bystander effect against non-modified, low-level drug resistant cells (FIG. 5, and FIG. 3 red cell/line). This eradicates drug resistance populations regardless of their molecular origins because the active form of the drug will kill cells in an EGFR independent manner (FIGS. 3 and 5).

FIG. 5 is a deeper tutorial on the success behind the data from FIG. 3, and an introduction to the use of stochastic models of evolutionary dynamics for selection drive design. A dimerizer is dosed alongside the initiation of targeted therapy (FIG. 3 right, FIG. 5, left). Upon introduction, a small molecule dimerizer (rimiducid in switch 1, FIG. 5, effect shown in FIG. 3 right) is administered. The selection gene drive senses the dimerizer concentration and performs an analog computation through biphasic activation. While the bulk of the non-modified population (blue cells in FIGS. 3 and 5) are being killed by targeted therapy against endogenous EGFR or ALK (T in FIG. 5), evolutionary pressure drives a population increase in cells harboring the selection drive (green cells in FIGS. 3 and 5). It was demonstrated that control of EGFR dependent growth dynamics for a dual-switch selection drive in EGFR transformed BaF3 cells is achieved. Some specific details on stochastic models of evolution to improve drug design are provided in the FIG. 5 description.

To address the molecular design goals of a selection gene drive approach more broadly, a stochastic model of evolutionary dynamics was created that includes all probable evolutionary failure modes. (FIG. 5, right) The cellular evolutionary dynamics of the system are analyzed at evolutionarily relevant population sizes. A system of 8 birth-death-mutation ODEs are parameterized that include a carrying capacity and parameterization for the suicide gene bystander effect. Importantly, mutation driven failure of every synthetic part and the targeted therapy is modeled. Arrows of a color in a model block diagram correspond to drive failure and mutation. One key observation is that it is best for selection gene drives should achieve an intermediate fitness level relative to pre-existing drug resistance (FIG. 5, modeling panel, bottom). This is because the stochastic risk of suicide gene failure increases if gene drive populations grow too big too fast. Creating a drive with intermediate fitness allows for all the benefits of selection mediated guidance and mitigate the risk of mutational failures in large populations.

### Example 5: The “Dual Switch′ Gene Drives is Optimized for Non-Small Cell Lung Cancer

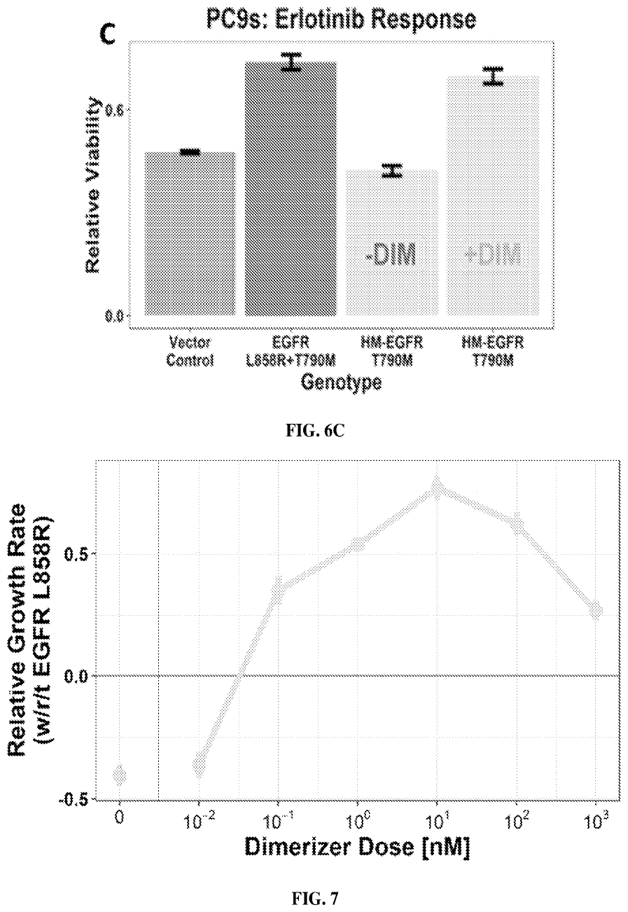

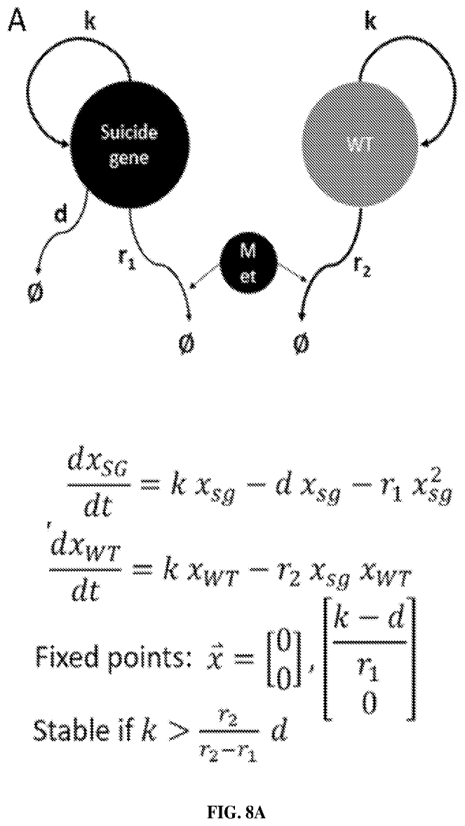

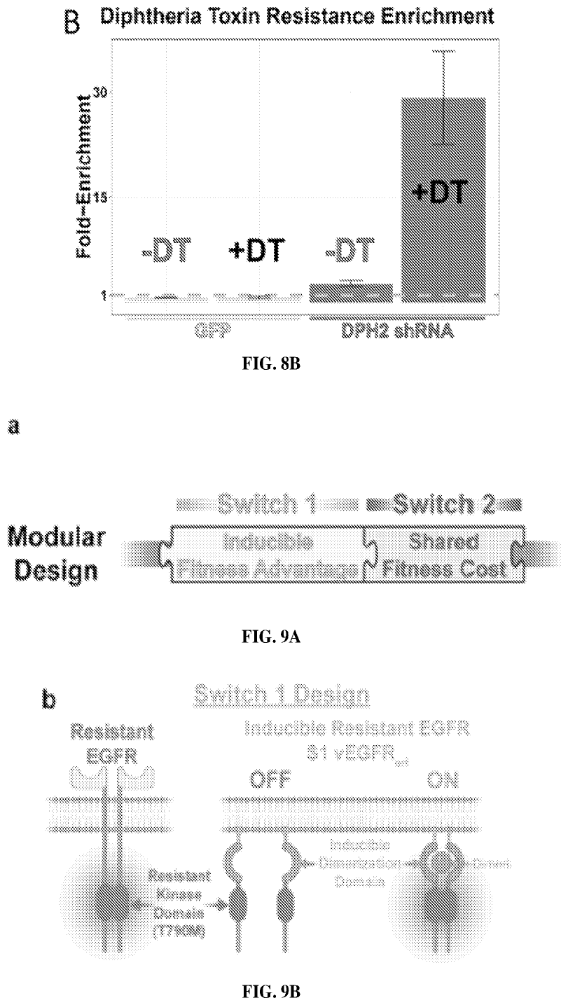

In addition to the POC data in FIG. 3, switch 1 also works well in NSCLC cell lines driven by EGFR (FIGS. 6 and 7). Prototypes of 3 distinct suicide genes (Cytosine Deaminase, NfsA, and Diptheria Toxin) have been validated with bystander effects in NSCLC cell lines (and a few non-NSCLC lines) that harbor clinically sensitizing mutations in the EGFR, ALK, ROS1, or RET oncogenes (FIG. 8, 2 shown). Importantly, switch 1 designs are ˜2-3 kB and Switch 2 designs are 0.5-1.5 kB and are easily compatible with lentiviral packaging in all of the permutations of this example.

The initial modeling shown in FIG. 5 below has identified 2 failure modes for which iterative engineering design is used to minimize the failure risks of a dual-switch selection drive. 1) Switch 1 must provide tunable intermediate fitness when sensing clinically relevant concentrations of the dimerizer in its local environment. 2) The active metabolite from switch 2 must efficiently kill a large proportion of cancer cells at pharmaceutically achievable prodrug concentrations.

Tuning a dimeric interface in switch 1 to match existing rimiducid clinical pharmacology. The clinical exposure of rimiducid is known to fall in the range of 0.1-1000 nM. The stochastic models showed that an important failure mode can occur when switch 1 creates too much fitness in gene drive cells (FIG. 5). One feature of chemical dimerization that aids this design is that the biophysics of binding create a natural safety valve for over-exposure to a resistance inducing dimerizer molecule. Instead of a sigmoid binding curve, the switch 1 binding kinetics shows a biphasic response to dimerizer molecules. Thus, increasing exposure to rimuducid can initially increase, but then subsequently decrease active switch 1 dimeric complex formation by promoting rimiducid-monomer pairs and therefore decreasing complex formation in vitro at high concentration. The designs in F36V-EGFR fusions in mammalian cells exhibit the precise biphasic response in vivo as shown in vitro (FIG. 7). The biphasic response peaks at 10 Nm. To broaden this bi-phasic peak into a range for patients, the kinetics of association are altered through non-cooperative multimerization of FKBP-F36V domains harboring various point mutations that reduce the affinity of the non-cooperative interaction. These additional domains (between 1-2) are used to maintain some of the safety features of a biphasic response, while extending and flattening the maximum concentration that is compatible with dimerizer function to values that contain the known Cₘₐₓ of rimiducid.

Twenty-seven candidate mutations adjacent to the FKBP12 interface have been identified by examining known structures. These residues are solvent facing and they make/support key contacts during chemically induced dimerization. Each mutation is made individually and in combination via gene synthesis. These constructs are cloned into a pooled library infected into EGFR mutant PC9 NSCLC cells and selected for the ability to grow in oscillating low and hi concentrations of rimiducid that is changed daily in the presence of an EGFR inhibitor. Rimiducid oscillation and samplings at multiple timepoints will improve hit calling by increasing signal to noise. Following selections, FKBP12 is sequenced, and candidates will be ranked and then validated for altered biphasic responses as shown in FIG. 7.

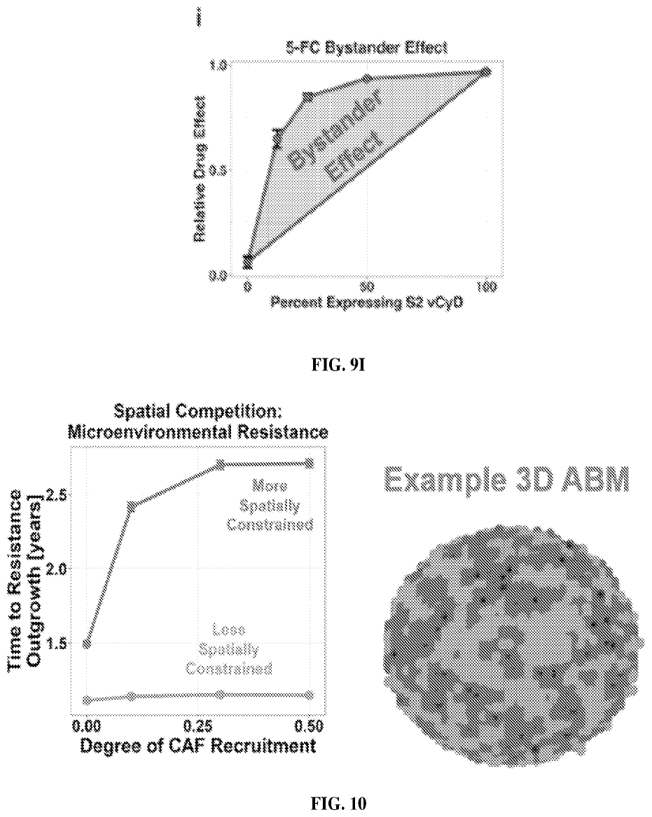

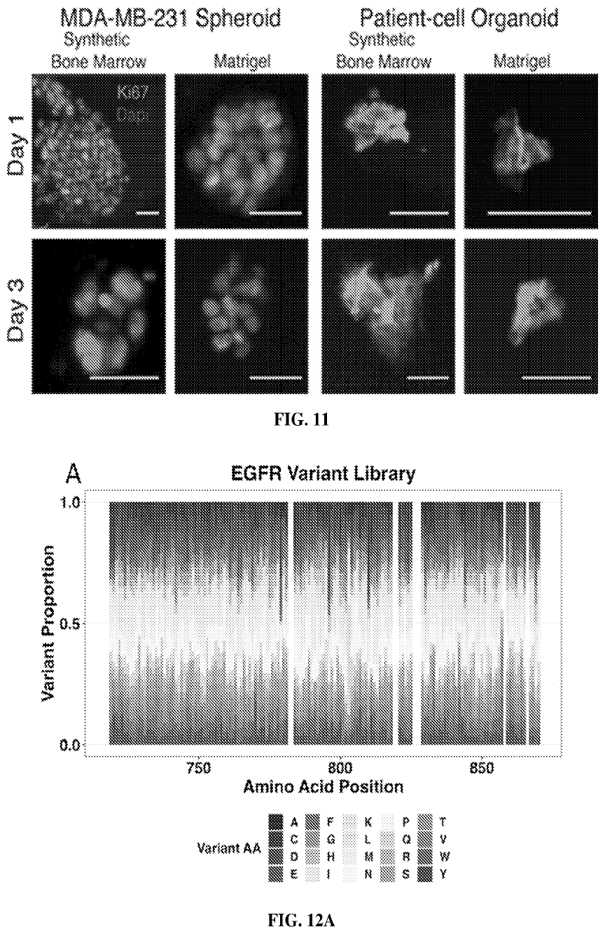

Personalize switch 2 designs for different NSCLC tumors. Quantitative models (FIG. 5) have identified the extent of switch 2 bystander driven killing as a key factor in gene drive success. This effect is larger in from spatial systems (2D/3D) (FIG. 10). Pharmacologically, the toxin responsible for the bystander effect must be diffusible and have a half-life on the order of hours to be evolutionarily useful. Prototypes used here meet these transport constraints, but some cancer cell types display differential biological sensitivity to distinct switch 2 metabolites. For instance, a RET fusion driven cancer cell line is highly sensitive to the cytosine deaminase->5-FC pair. While a PC9 cell line is more modestly sensitive to that cytosine deaminase>5-FC pair. Cell and organoid type specificity in Switch 2 designs is assessed in a larger number of genetically defined NSCLC cell line (FIGS. 8 and 10).

A panel included 1-3 example cell lines for multiple tyrosine kinases in NSCLC (PC9 (EGFR), H3122 (ALK), H1975 (EGFR), HCC827 (EGFR), H2228 (ALK), HCC78 (ROS1), STE-1 (ALK)) are used. All other tyrosine kinase driven cell lines are used as outgroup controls. Birth and death rates of active metabolites for all three currently validated switch 2 designs are measured in 2D and 3D spheroids. The most active switch 2 designs are used for each cell line. The Switch ½ pair is then personalized for a given kinase or cell line (i.e., EGFR/ROS1/ALK/RET) and for the most sensitive switch 2 design. Spike in experiments are then performed where pre-existing resistance mutants are labeled with mCherry (as in FIG. 3). Following lentiviral infection with personalized and non-personalized gene drive designs in the above cell lines, the dynamics of mCherry labeled resistant cells and gene drive containing cells are tracked over time. The evolutionary outcome of personalized and non-personalized designs are compared across all cell lines.

Switch 2 designs are built to harbor 2 tandem suicide genes for localized triple combination therapy. The small size of switch 2 genes (˜0.5-1.5 kb) means that 2 different suicide genes are stalled in the same lentiviral construct. This yields an approximately 6-7 kB packaging size and is within the limits of lentiviral infection. Having 2 switch 2's in the same construct has 4 benefits 1) It is an additional safety feature that provides redundancy. 2) A combination of 2 cell killing toxins mitigate cell line specific variation in switch 2 potency. 3) Active metabolites having additive killing efficacy in combination. 4) Existing mechanisms of suicide metabolites are independent and provide evolutionary benefits that reduce the probability of resistance. Switch 2 designs include gene directed prodrug therapies like CB1954, 5-FC, and Ganciclovir (pictured in FIG. 5), and transcriptionally inducible protein toxins like diptheria toxin, and Interferon gamma. Under the assumption that different cells have different optimal switch 2 combinations from these agents, the switch 2 toxins and designs in combination are examined. Active metabolites and recombinant proteins for switch 2 designs are dosed in a combination grid and interactions quantified using the method of Chou-Talalay. Combinations are either synergistic, additive, or antagonistic. Antagonistic combinations are eliminated from dual-switch 2 designs and synergistic combinations are prioritized. 10 combinations (5C2) are examined across 7 cell lines using the GRADE method. Top combinations candidates across NSCLC cells lines are prioritized based on CI values that are computed using the method of Chou-Talalay. The top 3 CI combinations initiate dual-switch 2 construction. Competition experiments with GFP labeled gene drive cells and mCherry labeled cells harboring pre-existing resistance mutants are followed by flow cytometry as in FIG. 3 and compared to the best single switch 2 designs.

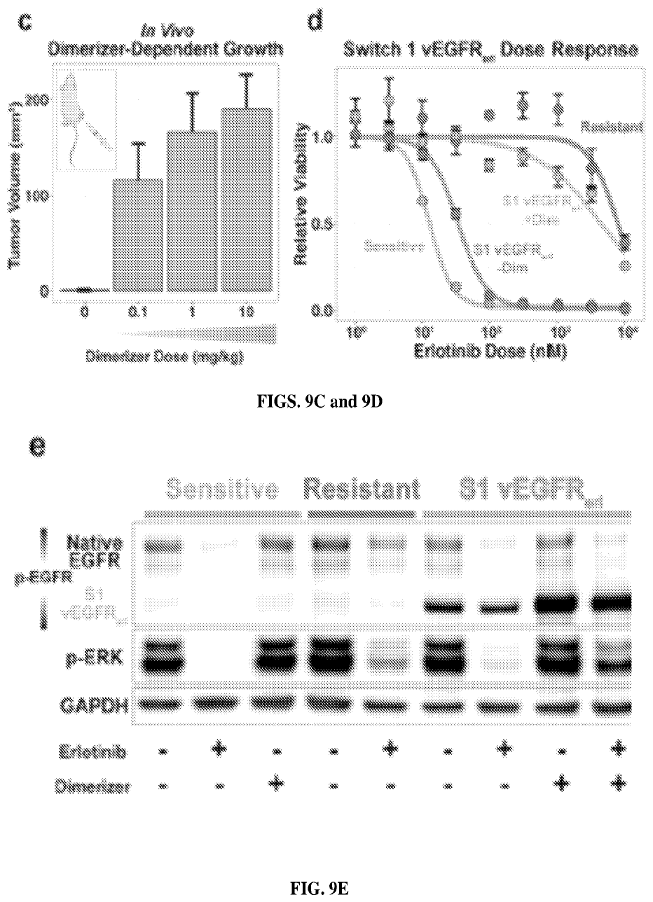

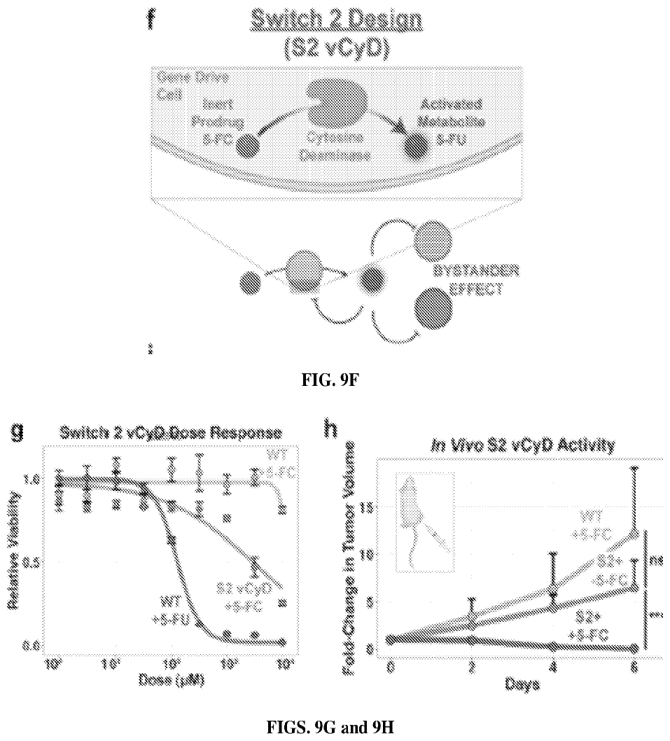

Switch 2 designs are built to harbor a second inducible resistance switch to boost local concentrations of suicide gene metabolites. Initial modeling of potential failure modes has shown that when gene drive cells are hyper-sensitive to a suicide gene metabolite they can die too quickly and produce a smaller bystander effect that kills fewer adjacent drug resistant cells. In fact, a controls analysis of the stability of gene-drive systems shows that no stable fixed points exist in the absence of transient resistance to switch 2 metabolites (FIG. 9). Transient resistance is a state of preserving a gene-drive cell “factory” until toxic metabolites have had a chance to accumulate in the local tumor microenvironment. If that drug factory waits to destroy itself until it has had enough time to export more of the bystander effect and kill adjacent drug resistant cells, then the design can be even more effective. Transient resistance is achieved in at least 1 switch 2 design through an inducible shRNA (takes up<200 bp and no need for Cas9). This shRNA targeting DPH2 protects against Diptheria Toxin (DT) in NSCLC PC9 cells. The small shRNA size makes it possible to transiently induce switch 2 resistance during DT release to maximize bystander killing by promoting secretion. FIG. 8B shows 30-fold resistance to a Diptheria toxin suicide gene constructed from an inducible shRNA. This improves mathematical stability by increasing the value of k, increasing k means that there is a stable fixed point where gene drive cells can maximize their switch 2 output to kill resistant cells in the tumor bed, regardless of the mechanism of resistance to EGFR therapy.

### Example 6: Defining Failure Modes of “Dual Switch” Gene Drives in Nsclc Cells Across Diverse Microenvironment Niches

Tumors are extremely heterogeneous, due to 1) unique mutations harbored by different cells, 2) many non-epithelial cells that comprise the tumor, and 3) the ECM in and near the tumor. This makes it critical to determine whether dual-switch selection drives maintain a fitness advantage across these separate axes of heterogeneity. Initial 3D Agent Based Models (ABMs) of spatially constrained competition was created between gene-drive drug resistance and microenvironmentally mediated survival. At the extremes in a heterogeneous microenvironment, spatial competition can synergize with microenvironmental stimuli to delay the outgrowth of a dual-switch selection drive (FIG. 10). This occurs because the microenvironment can temporarily outcompete strong genetic resistance when gene drive cells do not have sufficient space or resources to divide.

Switch 1 provides a selective advantage in the face of microenvironmental resistance that stromal cells and cell-ECM interactions provide to non-gene drive infected cells in the face of spatial competition. Switch 2 provides a reasonable safety switch to control oncogenic toxicity in the numerous non-cancerous stromal cells in and around the tumor. The switch designs are tested in four ways. 1) Calibrate on-lattice agent-based models of spheroid/organoid growth and treatment. 2) Determine whether a tradeoff occurs for microenvironmental-driven inhibition of dual-switch selection drives, whereby genetic resistance within the tumor, the degree of spatial constraint, the degree of ECM resistance, the assemblages of other cell types, and pharmacologic heterogeneity alters gene-drive action. 3) Identify new design goals by simulating in silico assemblages of tumors consisting of diverse genetic compositions, cell types, and ECM compositions and performing experiments to confirm simulations. 4) Gain concrete insights into design constraints of evolutionarily guided cell therapies. Analogous to failure analysis in engineering design, instances where the gene drive design fails across microenvironmental changes in the tumor are searched. A categorically successful design means that failures are only discovered at values irrelevant to the tumor microenvironment.

3D agent-based model of dual-switch selection drive is calibrated for treatment and resistance in NSCLC cell lines. Agent-based models were parameterized using data on heterotypic cancer spheroids. Resistance mutations and microenvironmental cues form competitive gradients are magnified by properties of the ECM (FIG. 10). A dual-switch selection drive must therefore work in a complex spatial environment and effectively compete with endogenous growth stimuli from stromal cells and cell-ECM signaling.

Tumor growth is modeled as a stochastic birth-death-mutation process on a lattice using the Gillespie algorithm to decide time steps and event identity. ECM- and stromal cell-driven resistance is modeled as an ensemble of N randomly seeded 3×3×3 sublattices that have different probabilities of birth/death in the propensity vector. These birth-death probabilities can be parameterized for genetically resistant PC9/H3122 cells, sensitive PC9/H3122 cells, PC9/H3122 cells harboring dual-switch gene drives, and stromal cell populations. As a first step, the birth and death rates of homogenous spheroids is measured in the lung ECM, as well as on plastic as a control. 3D hydrogels can be formed in 96-well plates using liquid handling robotics to allow for high-throughput screening (FIGS. 2 and 4). At 4-hour intervals for 96 hours, live and dead cells are quantified using a SYTOX based assay, which was optimized for kinetic analysis of drug response in a fluorescence plate reader with robotic plate handling and 3D spheroids.

To incorporate spatial competition between clones, cell division in this model is allowed when a space on the lattice is vacant. Like other models, “budging” is allowed to push cells along a lattice and accommodate division. However, the budging distance (measured in cells) determines spatial competition. Beyond a key parameter, the stiffness and degradability of the lung ECM environment used is controlled and allows spatial constraints to be tuned. Moreover, stromal cell competition is also controlled in these spheroids by changing seeding densities. The agreement between budging constraints in the model and spheroid growth rates can be tested. Briefly, 4 different stromal cell seeding density ratios are tested and compared to the tumor spheroids, from no stromal cells to 10% stroma, to 25% and 50%. The ECM conditions tested are the full lung ECM design (FIG. 4), plus a lung ECM that cannot be degraded by cells (thereby allowing cells to survive but not have sufficient space to divide), and a lung ECM that is 10× stiffer than normal, allowing only slow degradation. Oxygen diffusion and consumption can also be treated for larger spheroids by assuming 0.22 mM (partial pressure of 100 mmHg) in tissue culture media, a diffusion rate of 2×10⁻⁵ cm² s⁻¹ and a max consumption rate of 5×10⁻⁹ mol cm³ s⁻¹. Conventionally, diffusion is fast relative to division and solve the reaction-diffusion system numerically as the ABM updates using the method of successive overrelaxation with Chebyshev acceleration. The calculated concentration is then used to set a critical concentration for necrosis, death, or quiescence and interpreted as a rule by the ABM.

Spatial models calibrated and tested in experimental hydrogels. Birth and death rates of gene drive constructs are directly measured. Agent based models are straightforward to parameterize with these measurements (FIG. 10). Hydrogels of variable stiffness, degradation properties, and matrix compositions are created that model 3D spheroid and organoid growth. Before utilizing this model to identify failure modes of dual-switch selection drives, the models for matrix stiffness and degradability are calibrated in the absence of stromal support cells. These parameters should regulate the degree of open sites that are available for spheroid/organoid growth. This ability for cells to move and accommodate division within the ECM is modeled by a “budging” criterion that controls how a tumor can fill empty space. Because the budging criterion governs spatial competition, the direct calibration of this parameter is required for each gel with a variable number of MMP-degradable sites. These birth and death rates are systematically calibrated in the presence of targeted therapy, dimerizer, and the effects of switch 2 bystander killing (4*3*3*3 experiments in triplicate in 2 cell lines for 648 gels). The budging parameter (an integer distance in the lattice) is directly calibrated in degradable gels by predicting the bulk growth rate of untreated spheroids in the absence of any microenvironmental heterogeneity (i.e., the lung ECM gel with no changes as an initial control condition). In silico growth rates are simulated for 1000 spheroids up to ˜150 μM for each budging integer value. In vitro measurements of spheroid growth rates are then mapped in vitro in the lung ECM to identify the budging parameter that best matches the bulk growth rate for individual gels to use in subsequent models. Two cell lines (EGFR driven PC9 and ALK driven H3122), 4 ECM conditions, +/−stromal cells, +/−relevant lung MMP sites are followed during time lapse microscopy to see how migration distances compare to budging distances.

Birth and death rates calibrated and tested for genetically mixed populations and testing failure modes. The birth and death rates before and during therapy are not affected by mixed assemblages of genetically wildtype and resistant cells. Genetically resistant cells harbor no fitness defects. Models are built that use homogenous spheroid parameters, and contain entirely sensitive, or entirely resistant, or entirely gene drive containing cells. For pure populations, birth and death rates for individual cells are measured directly from 3D SYTOX in gels. Parameters derived from these pure populations are used to seed in silico mixed populations of genetically resistant and genetically sensitive cells across all ALK/EGFR cell lines. After simulation, physical mixtures of all cell lines are created in vitro, where 1 and 10% of the tumor is seeded with pre-existing EGFR or ALK resistance mutations (either T790M or L1202R). The measured bulk rates of birth and death for the mixed population by SYTOX is compared to the rates that are produced in silico. If large deviations occur, interaction terms for our models by microscopy of both cell lines at 1 mixture condition before examining failure modes with parameter sweeps are parameterized.

Microenvironmental toxicity modeling in tumor associated fibroblasts, endothelia, immune cells, and epithelial cells. Here, the transformation mediated toxicity in the stromal compartment are tested. The growth rates of stromal cells during gene drive treatment is measured. Tumor-stromal interactions using these growth rates are modeled to look for evidence of toxicity in spatially constrained and unconstrained tumors. The tumorigenic transformation of stromal cells is tested at the end of treatment by doing long term tumorigenesis assays in synthetic ECM. Long-term growth of 5 types of non-cancerous cells (lung fibroblasts, cancer derived fibroblasts, alveolar epithelial cells, pulmonary microvascular endothelial cells, and PBMC) are infected and examined for anchorage independent growth (a common metric of tumorigenesis) in soft hydrogels.

Computational testing of pharmacologic heterogeneity in the tumor bed. While it is challenging to spatially control concentrations of a targeted therapy, dimerizer, or suicide gene metabolite across a spheroid, a well parameterized model explores scenarios with heterogeneous intratumor pharmacokinetics and spatial gradients. Simulations that are sensitive to 3-fold variance in drug concentration can guide future design goals are used to test pharmacologic heterogeneity in tumors.

### Example 7: “Dual Switch” Selection Drive Prototypes are Directly Tested in Organoids from Egfr Mutant Nsclc Patients

Patient derived organoids are heterogeneous assemblages of genetically diverse tumor cells and stromal cells. These models recapitulate key aspects of the genetic heterogeneity and native tumor microenvironment in individual patients. They are also useful for evaluating the existing and novel therapeutics. The ability to grow organoids from many tumor types has been established as well as encase organoids in heterogenous ECM that resembles the lung. NSCLC is the most common human cancer, and EGFR mutations are present in 15-20% of all NSCLC patients. Thus, the generation of organoids is readily achievable with the current patient enrollment. Test the efficacy (through organoid growth assays) and safety (by looking for evidence of transformation of stromal cells in organoid culture) of dual-switch selection drives in NSCLC organoids is tested. Patient-derived ovarian cancer cells and breast cancer cells show that these organoids grow enough for these assays FIG. 11.

The ability of switch 1 designs to outcompete different genetic resistance mutations in organoids from EGFR mutant NSCLC patients. Switch 1 outcompetes resistance variants in heterogeneous organoids from diverse patients. Erlotinib resistance can be caused by T790M mutations in EGFR, amplifications in the c-MET oncogene, and activating mutations in other tyrosine kinases like RET and c-KIT. Lentiviral constructs expressing these genes/mutants subcloned into pLVX-IRES-Puro were obtained. mCherry is subcloned to replace puromycin resistance. Thus, mCherry labels pre-existing resistance mutations, as it does in FIG. 3. These mutations are introduced as a subpopulation into at least 5 different NSCLC organoids from patients with L858R or Exon19 deletion mutations in the EGFR oncogene that confer sensitivity to osimertinib and erlotinib. Resistant clones are tracked by mCherry as they compete with gene drive cells that are marked with GFP. All constructs are built and tested for expression and function in NSCLC cells. Constructs are packaged into viral particles. The organoids are infected, and the birth and death rates monitored. The spatial heterogeneity of gene drive cells are quantitated via automated microscopy at multiple time points. As a secondary measure of toxicity, non-cancerous cells are examined for gene drive infection mediated toxicity due to temporary transformation. Surface markers like CD31, CD34, CD45 and EpCAM can be used to monitor the growth of stromal cells of endothelial, bematopoietic, and epithelial origin by FACS. Toxicity mediated by non-cancerous cells can be definitively identified as normal cells by sequencing for the EGFR status from clonally isolated cells that are sorted into 96 well plates.

The sensitivity of organoids is examined from different patients to switch 2 metabolites. Since different cell lines have different sensitivities to distinct suicide gene metabolites, different patient derived organoids will have different responses to switch 2. This is supported by the variation of dose response in organoids for similar or identical compounds. Thus, the development path is to have multiple personalizable switch ½ combos and to match the right design to the right tumor via organoid testing. The overall workflow is summarized below.

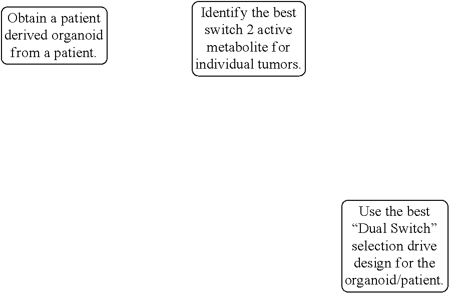

Organoid models are examined for sensitivity to all possible active metabolites and recombinant proteins package; 5-FU, CB1954, Diptheria toxin, IFNγ, Streptolysin O, and Ganciclovir. Because these molecules are the active products of switch 2 driven activation, switch 2 sensitivity in organoids is directly assayed without genetic modification and suicide gene induction. Note that some protein suicide genes are made inducible via an inducible promoter, not enzymatic activation. Dose response curves in vitro is performed for these organoids. An organoid version of a luciferase driven cell-titer Glo can be used to assay the number of live cells in these organoids. Then these sensitivities are used to determine the right suicide gene for the right organoid.

The coordinated action of switch 1 and 2 is examined in 5 EGFR+ patient derived organoids. After identifying the best switch 2 design for each of 5 different patient derived organoids, the coordinated action of a dual-switch gene drive with an EGFR resistant switch 1 and the switch 2 that is most active in that organoid is examined. This is a personalized version of the data shown in FIG. 3. A suspension of cells from an individual organoid is infected with a switch 1/switch 2/GFP construct. Note that in the case of protein switch 2, the induction is executed through inducible transcription, not enzymatic activation of a prodrug. These are mixed with a small number of mCherry modified resistant cells harboring the pre-existing resistance mutation T790M, activated MET or other mechanisms. Erlotinib is dosed at a range of clinically relevant concentrations given its Cₘₐₓ alongside 1-1000 nM rimiducid. GFP and mCherry populations are tracked by flow cytometry and microscopy. Success requires eradication of all transformed cells at pharmacologically relevant concentrations of small molecules.

The evolutionary risk in organoids with parameter sweeps of key variables. Taking birth and death rate measurements and models built allows parameterization of agent-based models (ABMs) with organoid relevant parameters. All ranges of birth and death rates are measured in patient derived organoids.

### Example 8: Programmed Evolution: Using Asexual Gene Drives to Sculpt Tumor Populations and Combat Genetic Diversity

Resistance evolution is the Achilles heel of targeted anticancer therapies. Tumor heterogeneity is so profound that pre-existing resistance is thought to be guaranteed at the time of disease detection. The practice of waiting for treatment failure in order to respond to resistance with next-generation therapies locks clinicians and drug developers in an evolutionary arms race until no further treatment options are available. Here, disease evolution is reprogrammed to design more readily treated tumors, regardless of the exact ensemble of pre-existing genetic heterogeneity. To program evolution, a genetic circuit composed of modular switches was conceived to develop asexual gene drives. Stochastic models of evolutionary dynamics were used to illuminate the design criteria of these “selection gene drives.” Prototypes were then built that perform according to these specifications in distinct cellular contexts and with diverse therapeutic mechanisms, including catalysis of a prodrug and induction of immune activity. Using saturating mutagenesis across a drug target and genome-scale loss-of-function libraries, selection gene drives are shown to eradicate profoundly diverse forms of genetic resistance. Finally, using theory to guide treatment scheduling, model-informed switch engagement is shown to create dramatic in vivo efficacy. These results establish selection gene drives as a powerful new paradigm for evolutionary guided anticancer therapy.

Drug resistance evolution represents one of the greatest challenges to the development of curative anticancer therapies. Studies of single cell heterogeneity have revealed that small resistant subclones often exist in the tumor at baseline, thereby guaranteeing treatment failure in most cases. Drug treatment dramatically reshapes the evolutionary landscape of the tumor microenvironment to select for these resistance variants. The result is outgrowth of a refractory tumor with fewer available treatment options.

Efforts to combat resistance are hindered by the intrinsic uncertainty of resistance evolution. In most cases, resistance variants are too rare to reliably detect at the beginning of treatment, and so the evolutionary trajectory of the tumor cannot be predicted. Thus, the conventional approach to treating resistance involves waiting for subclones to grow large enough to be clinically detectable, and then responding with an appropriate therapeutic strategy. In the case of targeted therapy, where resistance is commonly driven by point mutations in the target gene, this strategy often means developing and responding with next-generation inhibitors. For example, in EGFR+ non-small-cell lung cancer (NSCLC), the next-generation tyrosine kinase inhibitor (TKI) osimertinib is indicated for tumors treated with the frontline TKI erlotinib that have acquired a T790M resistance mutation. However, these next-generation therapies generally offer only temporary responses. The practice of waiting for primary resistance outgrowth during frontline therapy provides sufficient time and selective pressure to allow for the emergence of secondary resistance (FIG. 1A). In the case of EGFR+ NSCLC, erlotinib-refractory tumors treated with osimertinib often acquire the secondary resistance mutation C797S.

At the scale of the pharmaceutical industry, tremendous resources are invested in next-generation drug development to perpetuate this evolutionary arms race. At the scale of the individual patient, sequential monotherapy allows the tumor to evade each iteration of treatment until all available therapeutic options are exhausted.

Theoretical and empirical evidence shows that the only way to outpace resistance evolution is to employ combination therapies at the beginning of treatment. By combining agents with distinct mechanisms of resistance, the risk of cross-resistance is minimized. But the development of rational therapies that inhibit distinct oncogenic programs is fundamentally limited by the ability to identify new, orthogonal targets. Large-scale mapping of genetic dependencies in cancer have underscored the paucity of these therapeutically actionable genes. For example, in EGFR+ NSCLC, no secondary targeted inhibitors have led to standard-of-care combination therapies, despite repeated clinical efforts. Additionally, attempts to combine targeted EGFR inhibitors with more broadly cytotoxic chemotherapies have reported mixed results, likely due to the low therapeutic window of these agents.

Rather than search for new drug targets, alternative treatment strategies have sought to genetically modify cancer cells to artificially introduce exogenous, therapeutically actionable genes. Gene-directed enzyme prodrug therapy (GDEPT) involves introducing a “suicide gene” into cancer cells to locally activate an inert prodrug. The activated metabolite is generally diffusible, enabling GDEPT to target both modified and nearby, unmodified cancer cells.

However, clinical evaluations of suicide gene therapy have yielded underwhelming results, because poor gene delivery is a major challenge in GDEPT that precludes the eradication, even with the noted bystander activity.

Introducing exogenous drug targets is challenging, and sequential monotherapy ensures clinical efforts always remain one step behind cancer. The iterative approach of serial single-agent therapy resembles “reverse engineering” resistance evolution: after treatment failure has occurred, the nature of resistance is characterized, and an appropriate treatment response is tailored to it (FIG. 1A). Here, an alternative treatment strategy is presented that “forward engineers” evolution to redesign tumors that are more responsive to therapeutic intervention (FIG. 1B). This approach involves genetically modifying cancer cells in situ, then using small molecules to invert the tumor's evolutionary landscape to select for modified cancer cells in favor of resistant subclones. In re-engineering the tumor, drug selection can be exploited to generate more therapeutic opportunity, not less.

Inspired by CRISPR-based systems to control disease vector evolution, herein this approach is referred to as “dual-switch selection gene drives.” The genetic circuit is composed of two genes, or “switches,” that are stably introduced into cancer cells with a single vector. Switch 1 acts as an inducible resistance gene, endowing a transient resistance phenotype that amplifies the frequency of the engineered cells during treatment (FIG. 1C). Switch 2 is a therapeutic payload gene. As in standard GDEPT, it activates a diffusible therapeutic that kills both engineered and unmodified cancer cells. This bystander effect is maximized by hitchhiking on the Switch 1 gene. Importantly, this bystander activity is agnostic to whatever native resistant populations were being selected for during the Switch 1 phase of treatment. Additionally, because Switch 2 activity is limited to the tumor environment, higher local concentration of the activated agent can be safely achieved than would be possible through systemic administration. By granting more potent therapeutic action, the risk of cross-resistance is minimized and the promises of combination therapy may be fully realized.

Here, model-informed designs were used to construct and evaluate dual-switch selection gene drives for anticancer therapy. By engineering inducible drug target analogs, the controllable Switch 1 activity was demonstrated in multiple biological contexts. Moreover, therapeutic function and bystander killing for GDEPT and immune versions of the Switch 2 gene were established. The complete dual-switch circuits demonstrate the ability to eliminate pre-existing resistance, including complex genetic libraries within a drug target and across the genome. Finally, model-guided switch engagement demonstrates robust efficacy in vivo, highlighting the benefits of leveraging evolutionary principles rather than combating them. In total, these findings support the use of asexual gene drives rooted in evolutionary theory to re-engineer tumors and target diverse forms of native heterogeneity.

### Results

Theoretical models of asexual gene drives outline design parameters to successfully reprogram evolution. The selection gene drive system is a modular platform that couples an inducible fitness benefit with a shared fitness cost. Delivering and selecting for this genetic construct involves introducing more heterogeneity into a tumor population and intentionally expanding the genetically modified cancer cell population. To assess the mutational risks of this counterintuitive therapeutic approach, a stochastic mechanistic model of tumor evolution was developed. Such a model enables the anticipation and investigation of evolutionary risks associated with a selection gene drive system. Additionally, an understanding of the expected evolutionary dynamics under selection gene drive therapy can inform key design criteria. These criteria span important aspects of the system, including the gene delivery efficiency required to achieve evolutionary control and the fitness of gene drive cells in the Switch 1 treatment phase necessary to outcompete native resistance.

The model considers a small, initially sensitive population of cancer cells that expand until, upon tumor detection, a fraction of tumor cells is modified to become gene drive cells and treatment is initiated. The Switch 1 phase of treatment is maintained until gene drive cells become the dominant population, whereupon Switch 2 treatment begins. Over the course of the simulation, mutation events spawn subclones that model points of system failure. These mutations include acquired resistance to targeted therapy, resistance to the therapeutic action of the Switch 2 gene, and loss of Switch 2 activity among gene drive cells (FIG. 1D).

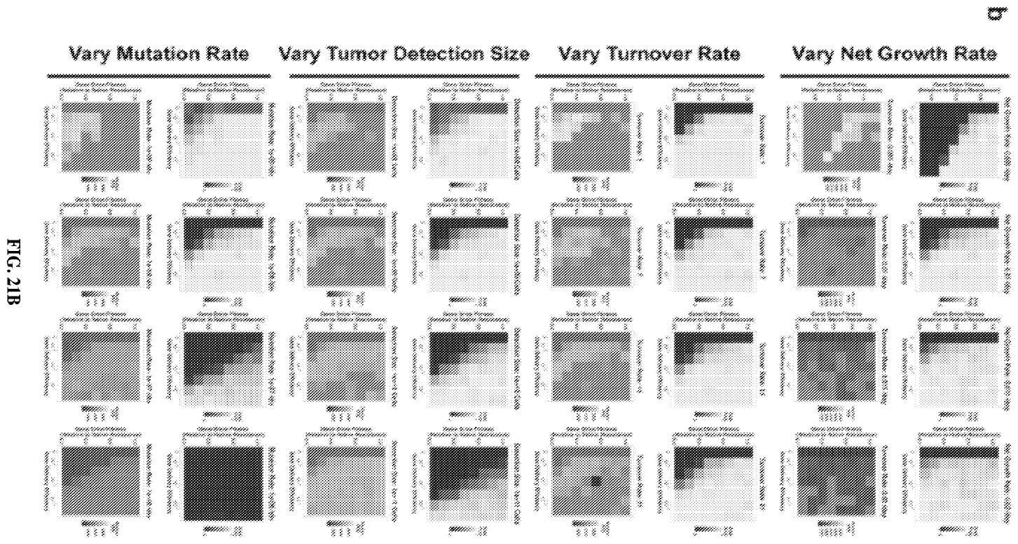

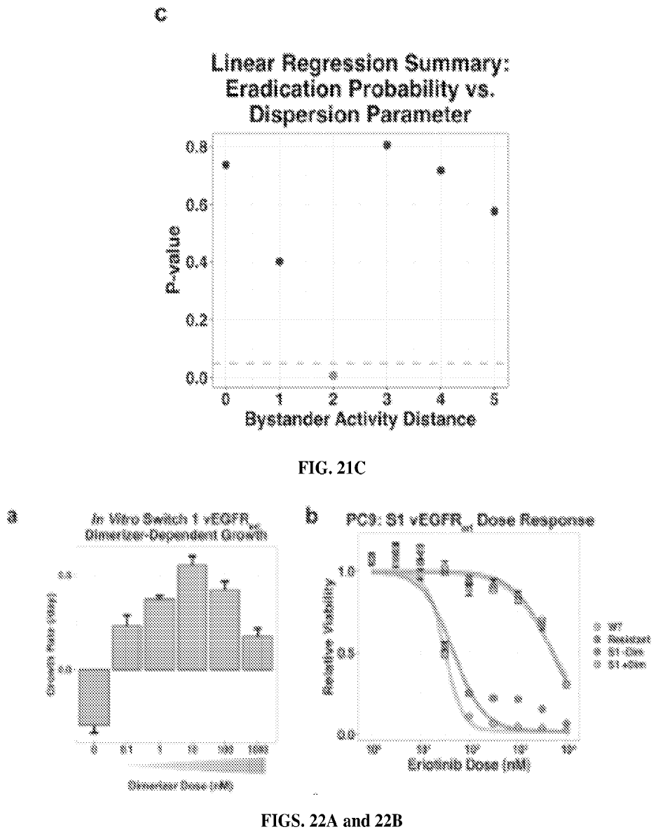

This system was simulated for a large range of model parameters. The evolutionary trajectory for one such simulation is shown in FIG. 1E. Analysis of simulation results indicates that gene delivery need not be very efficient. The model demonstrates that selection under Switch 1 can overcome limitations imposed by poor gene uptake, and evolutionary control is predicted to be possible for <1% initial gene drive population under some conditions (FIG. 1F). Additionally, simulation results show that evolutionary control is possible even when gene drive cells are less fit relative to native resistant populations. This is because, even with poor gene delivery of around 1%, the gene drive population is expected to be orders of magnitude more abundant that resistant subclones at the onset of treatment, allowing even low-fitness gene drive cells to outcompete. The evolutionary model also points towards optimal treatment regimens. In particular, simulation results highlight the benefit of some delay between the engagement of Switch 2 and the cessation of Switch 1 (FIG. 21A). Sensitivity analyses reveal that these findings are robust to variation in growth kinetics, but outcomes improve for smaller detection sizes and lower mutation rates (FIG. 21B).

In total, these modeling results explore many conceivable failure modes with physiologically plausible parameters and predict the outcomes for success in forward engineering tumor populations. While standard monotherapy is predicted to fail across all physiologically relevant conditions, simulation results indicate that selection gene drive therapy extends progression-free survival in all cases (FIG. 21B) and eradication in most conditions (FIG. 1F). Because the transient nature of the Switch 1 selection gene is sufficient for outgrowth under targeted therapy treatment, resistant subclones that spawn in the gene drive population during Switch 1 treatment are not expected to increase in abundance relative to base gene drive cells. Instead, across simulation conditions the unmutated gene drive the population to dominate the tumor environment. Then, Switch 2 exploits this dominance to clear both gene drive and natively resistant cells, before cross-resistance has an opportunity to emerge.

In addition to mutational points of failure, spatial risks of a selection gene drive system were assessed. In particular, the bystander effect of the therapeutic Switch 2 gene requires some proximity with unmodified cells in order to eliminate them. Therefore, the spatial distribution of gene drive cells and the range of bystander activity are important determinants of therapeutic success. To contemplate spatial sources of failure, a spatial agent-based model of the selection gene drive system was constructed. The model considers a mixed population of sensitive, resistant, and gene drive cells. While the initial spatial distribution of resistant cells is random, gene drive cells are seeded according to a spatial dispersion parameter (FIGS. 1G and 1H). Additionally, the distance over which the bystander effect acts is allowed to vary.

Model results indicate that the selection gene drive system benefits when bystander activity is diffuse (FIG. 11). However, simulation outcomes were relatively independent of the initial spatial distribution of gene drive cells. The spatial structure of the gene drive population was predictive of eradication probability only for a narrow set of conditions (when the bystander range is ˜2 cell lengths; FIG. 21C). Together, these results led to favoring of Switch 2 genes with diffuse activities, such as those of standard GDEPT systems over other therapeutic transgenes that require direct contact with their neighbors. Given the diffusion characteristics of most activated GDEPT prodrugs, a diffusible metabolite would exceed the design criteria of the agent-based model.

Modular, synthetic drug targets function as controllable “Switch 1” selection genes. The theoretical compartmental and agent-based models show that selection gene drives are an effective approach towards achieving evolutionary control, and so a genetic construct was designed and assembled to be guided by these results. A modular approach was prioritized to the gene drive design. The fundamental function of the genetic circuit is to couple an inducible fitness advantage (Switch 1) with a shared fitness cost (Switch 2; FIG. 9A), and so a number of orthogonal Switch 1 and 2 motifs were evaluated.

When designing the Switch 1 gene, an inducible version of a kinase drug target was engineered. Given that oncogenic kinase activity is often the result of constitutive dimerization, it was contemplated to controllably mimic oncogenic signaling by fusing the kinase domain of a drug target to a synthetic dimerization domain. Here, an FKBP12 F36V domain was used, which is designed to promote homodimerization in the presence of the small molecule dimerizer AP20187, which has engineered specificity for the F36V mutant over endogenous FKBP12-containing proteins. This system is attractive because a closely related inducible dimerizer has demonstrable activity and safety in human patients. The kinase EGFR was selected for an initial design. To generate an inducible version of EGFR, an FKBP12 F36V fusion was cloned to the juxtamembrane, kinase, and C-terminal domains of EGFR, which are required for activation of downstream signals. In addition, an N-terminal Src myristylation sequence was included to target the synthetic EGFR protein to the cell membrane. Finally, to rescue signaling of the EGFR analog in the presence of erlotinib (and thus enable selection under drug treatment), a resistance conferring T790M mutation was introduced (FIG. 9B). This initial design of an inducible resistant drug target was named “S1 vEGFRₑᵣₗ.”

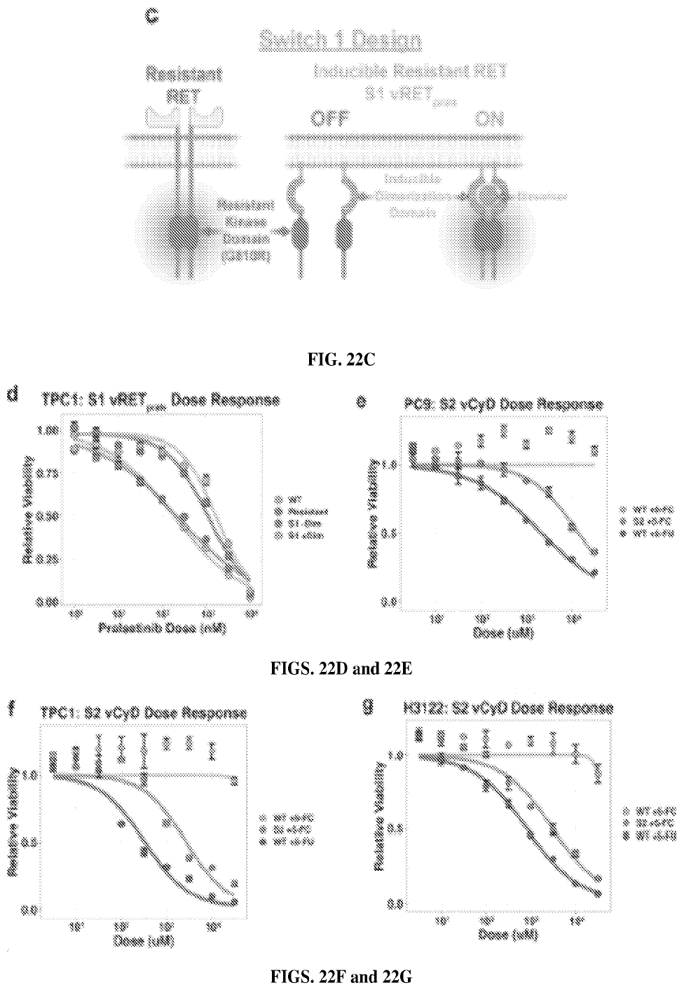

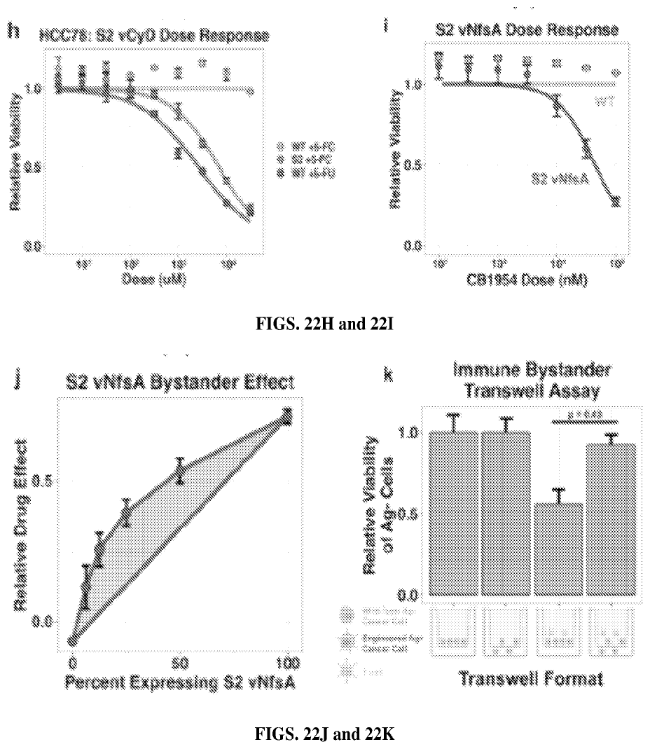

To evaluate the inducibility of S1 vEGFRₑᵣₗ signaling, this synthetic gene was expressed in BaF3 cells. In the absence of IL-3, the growth of S1 vEGFRₑᵣₗ BaF3 cells were found to be dimerizer-dependent, showing that this construct can controllably mimic native kinase activity (FIG. 22A). Stimulated growth was observed across a wide range of dimerizer concentrations, indicating that kinase function is robust to the precise dose of dimerizer. To test the activity of this Switch 1 construct in vivo, subcutaneous grafts of S1 vEGFRₑᵣₗ BaF3 cells was generated in mice. Once-daily administration of dimerizer stimulated tumor growth, showing that this system could inducibly mimic oncogenic signaling in vivo as well (FIG. 9C). Again, this activity was observed for a range of dimerizer doses, indicating that this Switch 1 gene could be robust to heterogeneity in pharmacokinetic profiles across patients.

To confirm the inducible resistance phenotype, the erlotinib dose response of S1 vEGFRₑᵣₗ BaF3 cells with an EGFR+ background was evaluated (FIG. 9D). In the absence of dimerizer, the erlotinib dose response of these cells reflected that of drug-sensitive EGFR+ BaF3s. However, in the presence of dimerizer these Switch 1 cells demonstrated erlotinib resistance. To assess the ability to induce resistance in a human cancer cell line, S1 vEGFRₑᵣₗ was expressed in the EGFR+ NSCLC PC9 cells. Indeed, the Switch 1 design conferred an inducible resistance phenotype in these engineered cells (FIG. 22B). Importantly, this inducible resistance phenotype was observed across several orders of magnitude of erlotinib concentrations.

To assess whether the synthetic S1 vEGFRₑᵣₗ gene faithfully recapitulates EGFR behavior, molecular signaling was characterized in PC9 cells. Western blots for phospho-EGFR and phospho-ERK indicated that erlotinib blocks native EGFR autophosphorylation and MAPK activity (FIG. 9E). However, the addition of dimerizer rescues MAPK signaling in erlotinib-treated S1 vEGFRₑᵣₗ PC9 cells. In total, these results show that S1 vEGFRₑᵣₗ effectively phenocopies native on-target resistance upon dimerizer administration.

Beyond EGFR, it was sought to develop Switch 1 motifs for another therapeutically actionable kinase: RET. RET fusions confer sensitivity to the RET inhibitor pralsetinib in NSCLC and thyroid cancers. Thus, an FKBP-RET fusion protein was generated with a pralsetinib-resistance conferring G810R mutation to develop S1 vRETprals (FIG. 22C). To evaluate the functionality of this orthogonal Switch 1 gene, RET+ TPC1 cells were transduced with S1 vRETprals. Indeed, these engineered cells were resistant to pralsetinib in the presence of dimerizer, and sensitive otherwise (FIG. 22D). This finding shows that the Switch 1 dimerization motif is generalizable to other targetable kinases.

Modular “Switch 2” motifs generate robust anticancer activity with bystander effects. Next, the design of the Switch 2 gene was considered. Guided by the results of the spatial agent-based model, therapeutic genes with diffuse activities were considered. For an initial Switch 2 construct, cytosine deaminase (S2 vCyD) was evaluated. Cytosine deaminase is an enzyme capable of converting the functionally inert prodrug 5-FC into the potent cytotoxin 5-FU (FIG. 9F). While shown to be safe, efficacy was limited by poor gene delivery. Furthermore, cytosine deaminase is an attractive Switch 2 gene because the prodrug 5-FC is an approved, well-tolerated antifungal agent, and the activated agent 5-FU is a well-studied chemotherapeutic with a half-century history of clinical evaluation across many cancer types.

Expressing an optimized S2 vCyD in BaF3 cells effectively sensitized them to 5-FC treatment (FIG. 9G). Furthermore, a panel of other human cancer lines engineered to express S2 vCyD exhibited similar levels of 5-FC sensitivity, showing general activity across different biological contexts (FIGS. 22E, 22F, 22G, and 22H). To assess the efficacy of the CD/5-FC system in vivo, EGFR+ BaF3 cells engineered to express S2 vCyD were grafted in the flanks of mice. Daily dosing of 5-FC resulted in rapid tumor regression, showing potent in vivo activity (FIG. 9H). While there was no significant difference in the growth rates of wild-type tumors and untreated S2 vCyD tumors (p-value=0.09), a fitness cost associated with cytosine deaminase expression is possible, even in the absence of 5-FC. Small growth defects have been reported in other enzyme-prodrug systems. However, the computational modeling herein shows that selection gene drives are robust to even large fitness defects (as low as 50% decrease in growth rate relative to native resistance, FIG. 1F). These results indicate that a small fitness cost associated with Switch 2 gene expression is tolerable.

To validate the bystander effect of the CyD/5-FC system, mixed populations of wild-type and S2 vCyD BaF3 cells were treated with 5-FC. In the absence of a bystander effect, 5-FC/5-FU activity is limited to S2 vCyD cells. Thus, the relative drug effect is proportional to the fraction of Switch 2 cells in a pooled population. However, 5-FC treatment in mixed cultures resulted in significantly higher killing than expected showing a strong bystander effect (FIG. 9I).

In addition to cytosine deaminase, the alternative suicide gene NfsA was evaluated. NfsA is an enzyme that converts the prodrug CB1954 into an activated nitrogen mustard species. An earlier version of this system has been clinically evaluated, but failed to demonstrate lasting activity, likely due to limits imposed by poor uptake of the therapeutic gene. Dose response assays confirmed that 293T cells engineered to express an S2 vNfsA construct were effectively sensitized to CB1954 (FIG. 221). In addition, assays in mixed cultures of wild-type and S2 vNfsA cells confirmed a strong bystander effect in this enzyme/prodrug system as well (FIG. 22J).

These results highlight alternative designs for the Switch 2 gene. Such alternatives may be useful, especially when targeting tumors with known recalcitrance to 5-FU treatment. Furthermore, because 5-FU and nitrogen mustard agents have distinct mechanisms of action, there may be utility in combining these genes to achieve a combination-version of Switch 2, with non-overlapping modes of failure.

Beyond the activation of a diffusible prodrug, alternative Switch 2 systems with novel therapeutic functions were considered. Previous studies have demonstrated that CD8+ T cells can exhibit on-tumor, off-target activity. This nonspecific cytotoxicity, mediated through FasL signaling, can kill both antigen-positive and antigen-negative cancer cells. It was contemplated that this activity could serve as a bystander effect in an immune version of Switch 2 (FIG. 9J). Therefore, PC9 cells were engineered to express CD19 (S2 vCD19). These engineered cells were co-cultured with wild-type cells in the presence of primary T cells and the bispecific CD3/CD19 engager blinatumomab. S2 vCD19 cells were targeted by the T cells (FIG. 9K). However, wild-type, antigen-negative cells were affected as well, indicating immune bystander activity in this system.

To test that the observed effect on wild-type, antigen-negative cells was due to depletion of resources in the media caused by T cell expansion, bystander activity was tested in transwell plates. This experimental format physically separates the antigen-positive and negative cells but allows for the sharing of resources between the two populations. Results confirmed that the depletion of wild-type cells required direct contact with T cells (FIG. 22K), indicating that the observed bystander effect was not an artifact of culture conditions. These results point toward activating the immune system to generate a bystander effect in an immune version of the selection gene drive system. While CD19 was used here as an initial proof-of-concept, an engineered, orthogonal tumor-specific antigen could function as a Switch 2 immune target. Such a system involves modifying cancer cells to express this antigen, selecting for the modified, antigen-positive population, and then engaging the immune system to clear both antigen-positive and antigen-negative cells. T cell tumor infiltration and migration provides a long-distance bystander effect, satisfying the design criteria established by the spatial agent-based model (FIG. 1I). Furthermore, activation of the immune system has been shown to have an abscopal effect. By encouraging interactions between immune cells and immunogenic dying cancer cells, an immune selection gene drive enables the immune system to identify endogenous tumor neoantigens. Such activity directs immune cells to recognize and eliminate cancer cells at distant metastatic sites that are difficult to target with localized gene delivery.

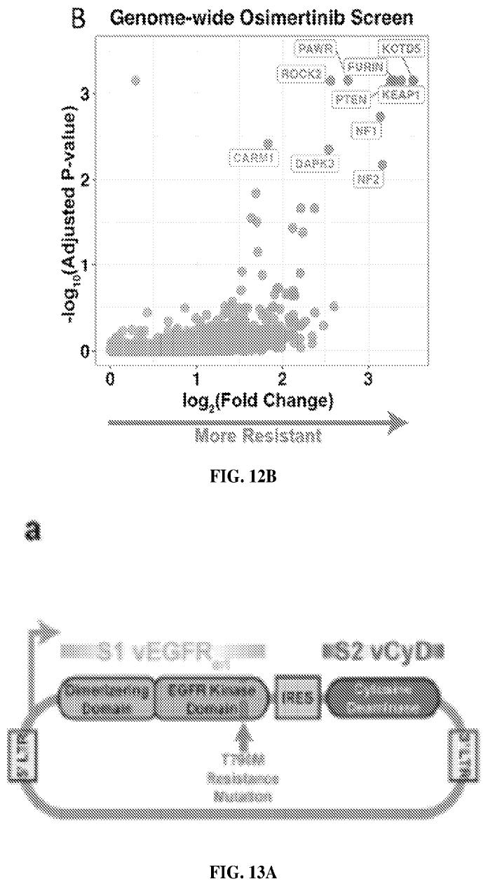

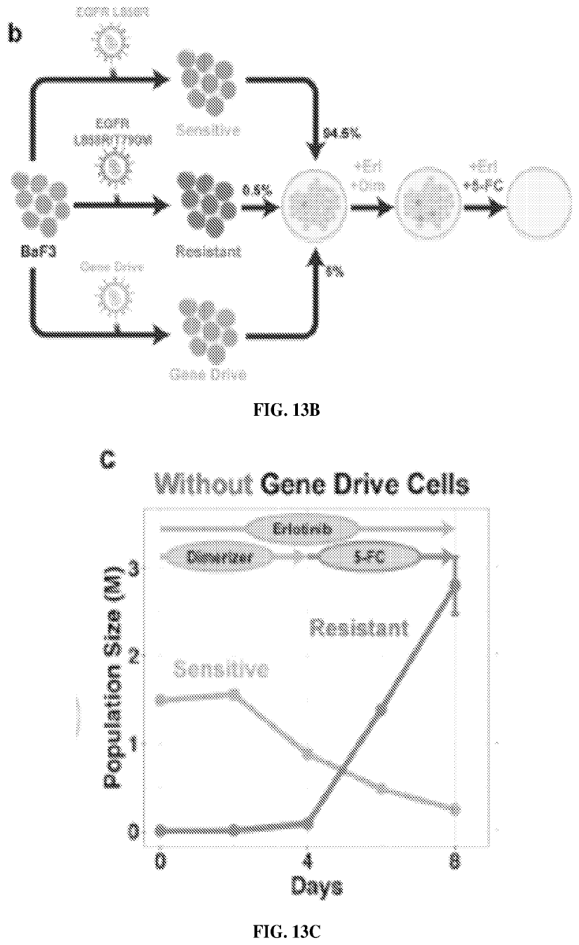

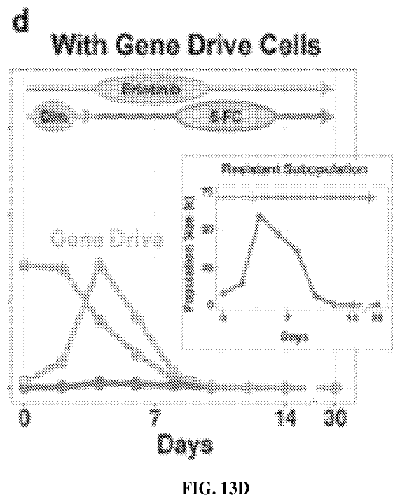

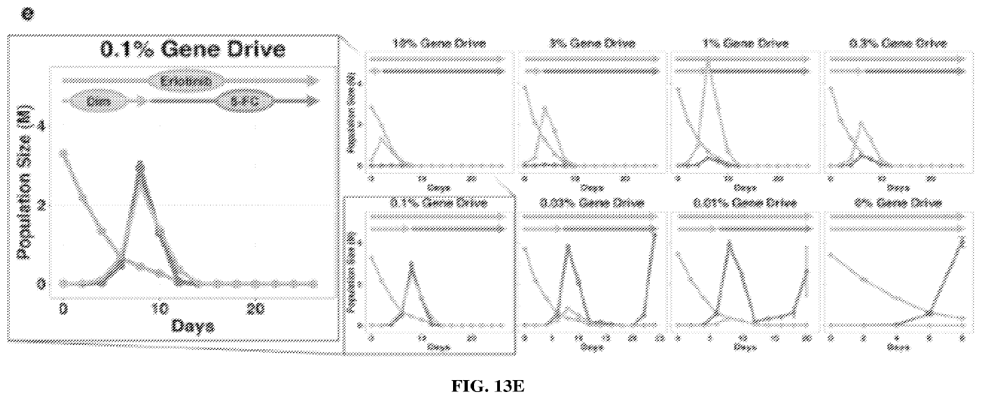

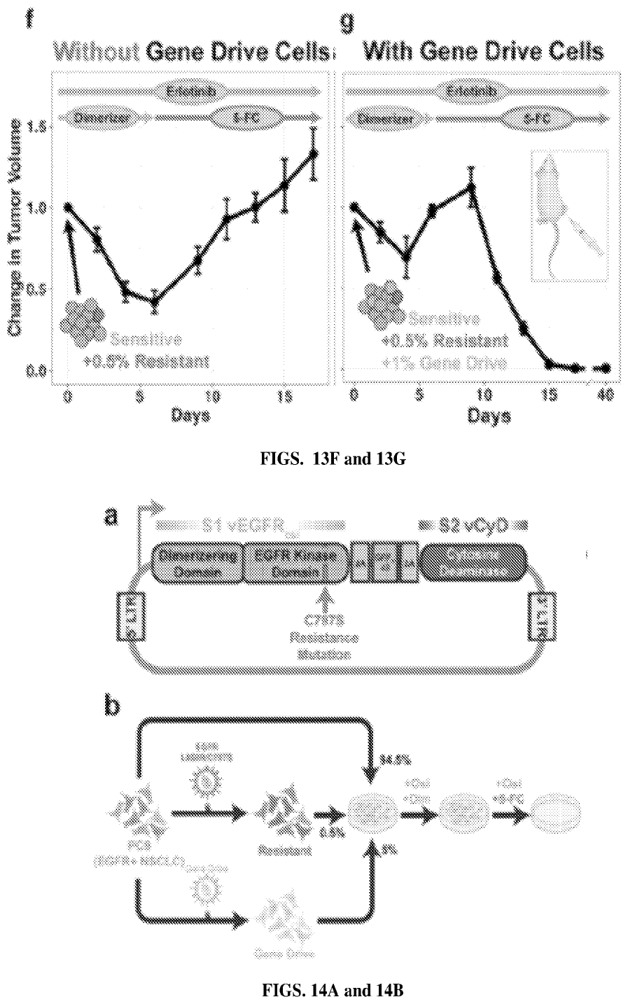

Integrating both switches into a single system couples an inducible fitness benefit with a shared fitness cost. Having established the activity of the Switch 1 and Switch 2 genes in isolation, their functionality was evaluated in concert. The S1 vEGFRₑᵣₗ and S2 vCyD genes were cloned into a single vector (FIG. 13A) and expressed in EGFR+ BaF3 cells to generate a complete gene drive system. To track the growth dynamics of subpopulations in mixed cultures, gene drive cells were engineered to express GFP and resistant cells to express mCherry. Populations of sensitive, resistant, and gene drive BaF3 cells were then pooled (FIG. 13B). The gene drive cells were seeded at 5% of the total population, reflecting a more modest gene delivery efficiency than has been demonstrated in the clinic. The resistant subpopulation was seeded at 0.5% abundance, which is orders of magnitude larger than the resistance frequency predicted by theoretical studies and clinical measurements. Thus, this population structure represented a conservatively challenging context to evaluate gene drive performance.

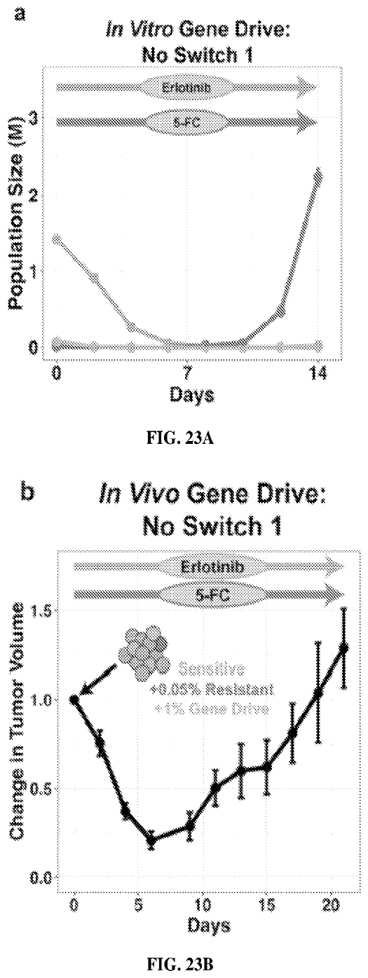

In treating mixed populations of only sensitive and resistant cells, the bulk sensitive population regressed while the resistant population expanded, mirroring the dynamics of relapse observed in the clinic (FIG. 13C). However, the addition of gene drive cells enabled the selection of this engineered population under dimerizer treatment, in place of the native resistant cells (FIG. 13D). Once the gene drive cells became dominant, 5-FC treatment was initiated. The gene drive population quickly collapsed, along with the resistant population. Monitoring these cultures for 30 days revealed no outgrowth of cells, indicating complete elimination of the spiked-in resistance. Importantly, treating this same population structure with erlotinib and 5-FC at baseline (i.e. Switch 2 treatment only, without an initial Switch 1 selection phase) failed to clear the resistant population, thus highlighting the necessity of Switch 1 activity (FIG. 23A). These results show that selection gene drive therapy can be used to clear mixed populations that would otherwise relapse under monotherapy.

This evolutionary model shows that the selection gene drive system is robust to inefficient gene delivery. To test this finding empirically, mixed populations of sensitive and resistant BaF3 cells (0.1% resistant population) were created and the spike-in of gene drive cells were titered to reflect poor uptake of the genetic construct. The eradication of these mixed populations shown for baseline gene drive frequencies as low as 0.1% (FIG. 13E). Given that the resistant subpopulation used here (0.1%) is generally much more abundant than in real-world tumor populations, this in vitro result shows that selection by Switch 1 can be used to overcome remarkably low gene delivery efficiency and maximize bystander activity against competing subpopulations. Even in conditions where the resistant population eventually came to dominate (0.01-0.03% gene drive spike-in), Switch 2 bystander activity was sufficient to delay resistance outgrowth relative to the no gene drive control condition (FIG. 13E). Thus, gene drive therapy may have utility, even in cases where complete evolutionary control cannot be achieved.

Given the spatial aspects of the gene drive system, it was next sought to evaluate gene drive behavior in a 3-dimensional context by transplanting mixed populations of BaF3 cells in mice. After tumor establishment, mice were treated daily with erlotinib and dimerizer. Upon disease progression, dimerizer was replaced with 5-FC treatment. As in the in vitro case, tumors lacking gene drive cells initially regressed and then relapsed, indicating that they had become refractory to erlotinib (FIG. 13F). Furthermore, tumors with gene drive cells that were treated with erlotinib and 5-FC at baseline (i.e. no initial Switch 1 phase) exhibited similar growth dynamics, showing that, in the absence of selection, gene drive cells could not clear the resistant population (FIG. 23B). Rather, the gene drive cells were likely depleted before they could generate enough bystander activity to eliminate the resistance subpopulation. However, in tumors with gene drive cells that received an initial dimerizer regimen, the “relapsed” tumor was highly sensitive to 5-FC treatment, showing successful reprogramming of the tumor population (FIG. 13G). These mice were monitored for 40 days, and no tumors redeveloped.

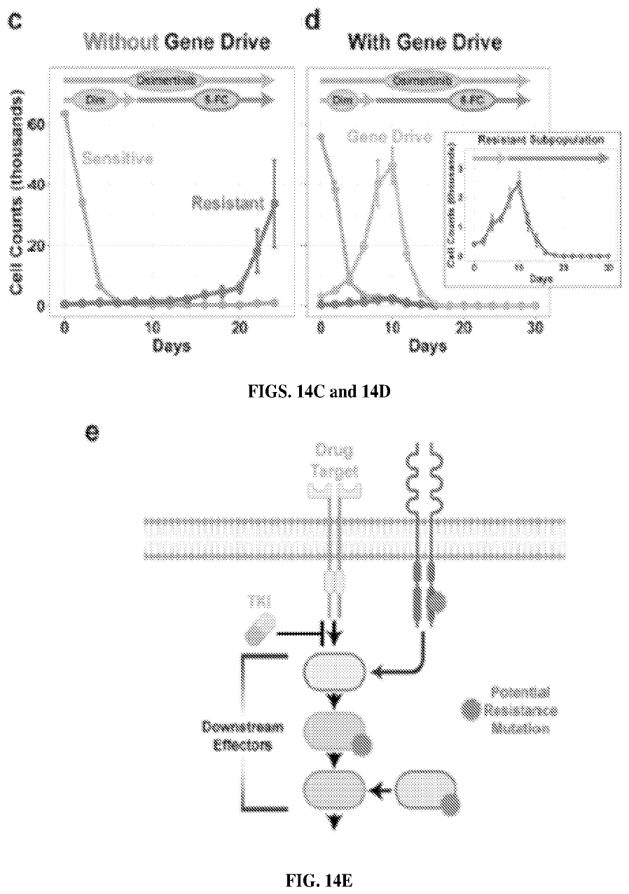

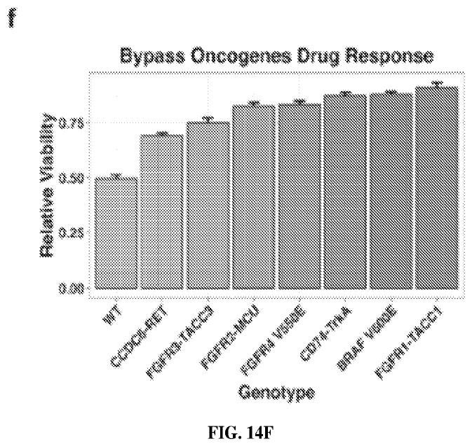

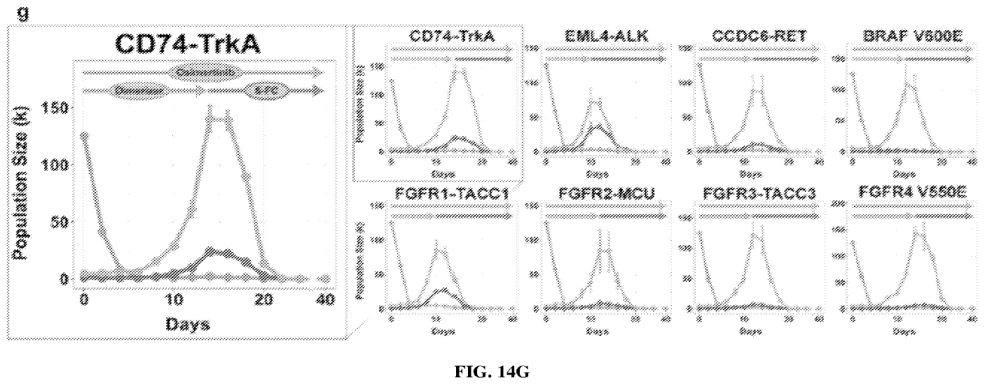

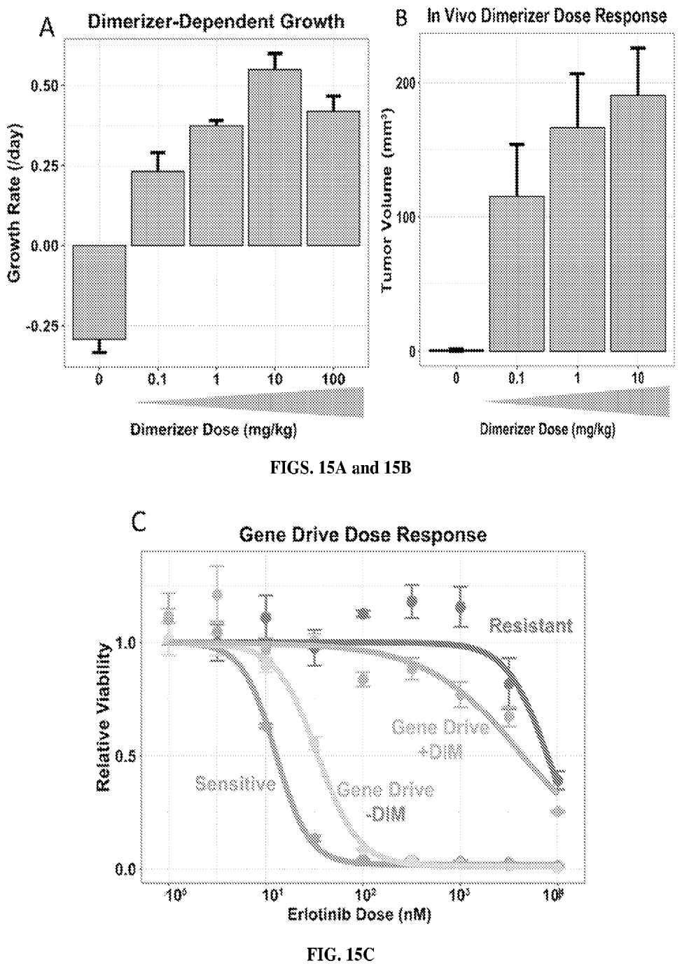

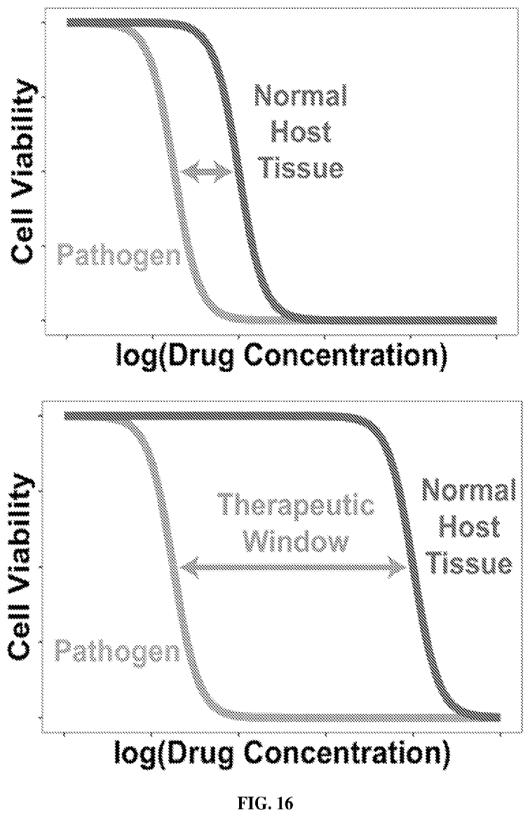

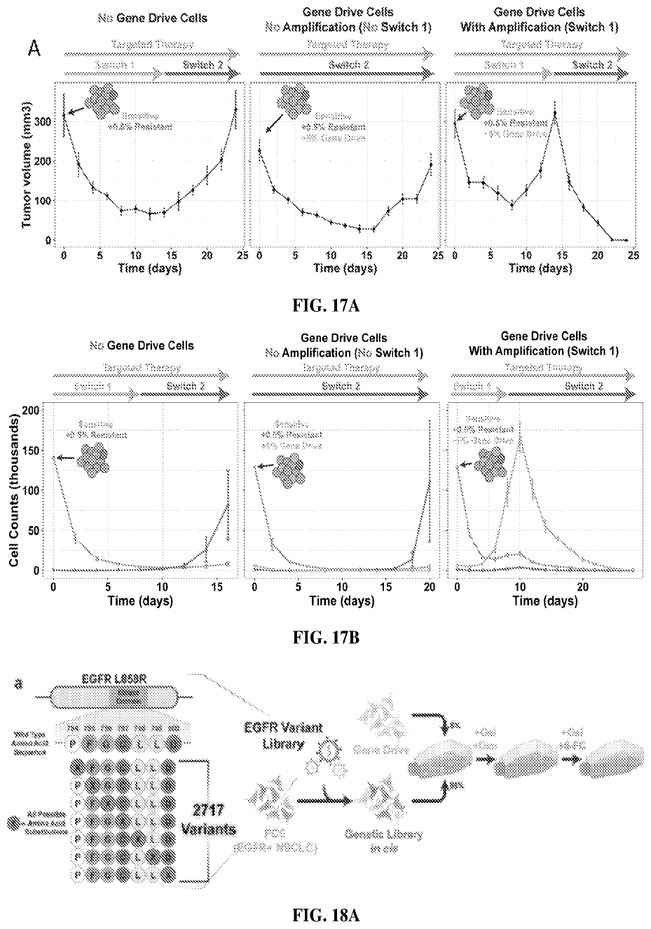

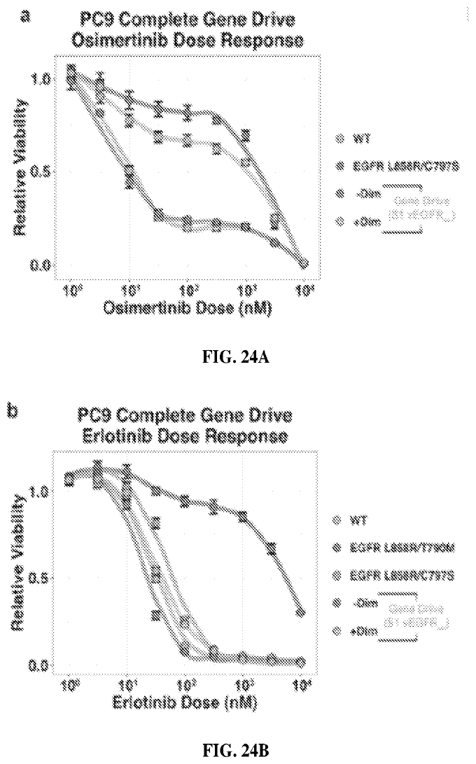

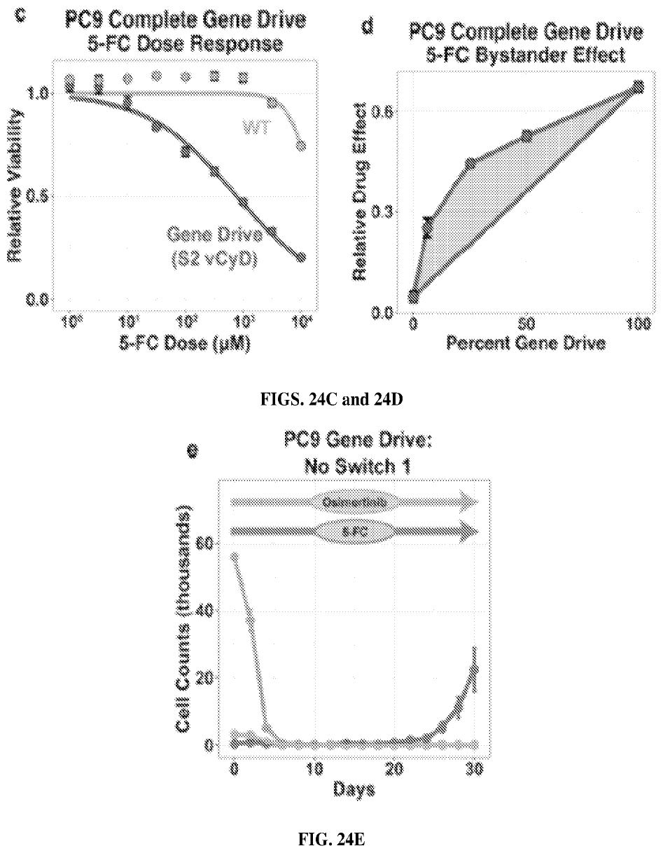

Evolutionary reprogramming eliminates pre-existing resistance in an NSCLC model. With proof-of-concept established in BaF3s, the complete gene drive system was evaluated in human cancer cells. Given that frontline use of the third-generation EGFR inhibitor osimertinib has demonstrable superiority over first-generation inhibitors in the clinic, an osimertinib-compatible version of the gene drive system was developed. Thus, the T790M erlotinib-resistance mutation was replaced with a C797S osimertinib-resistance mutation (S1 vEGFRₒₛᵢ). This updated Switch 1 gene and S2 vCyD was cloned into a single genetic construct (S1vEGFRₒₛᵢ-S2vCyD; FIG. 14A). This design also included a split GFP system to enable monitoring of gene drive cells in mixed populations. Osimertinib dose response assays among PC9 cells transduced with the complete gene drive system confirmed the inducible resistance phenotype (FIG. 24A). Notably, these cells retained their sensitivity to erlotinib even when Switch 1 is engaged by dimerizer treatment (FIG. 24B). These results support the use of alternative TKIs as a fail-safe against gene drive subclones that acquire constitutive Switch 1 activity in clinical applications. PC9 cells expressing the complete gene drive construct also demonstrated sensitivity to 5-FC (FIG. 24C) and bystander activity (FIG. 24D).

In growth tracking experiments (FIG. 14B), osimertinib treatment of pooled populations without gene drive cells ultimately selected for C797S resistance (FIG. 14C). However, in mixed cultures with a spiked-in gene drive population, selection for this engineered population with dimerizer treatment, followed by eradication of all cells by administering 5-FC was demonstrated (FIG. 14D). Here again, it was observed that the Switch 1 treatment phase was required to eradicate resistance (FIG. 24E).

Point mutations in the drug target gene (e.g. C797S) represent only one mode of treatment failure that tumor cells can exploit to evolve therapeutic resistance. In NSCLC patients treated with frontline osimertinib, 22-39% of tumors acquire mutations or fusions in genes parallel to or downstream of EGFR. Activation of these genes serves to maintain oncogenic signaling, even when EGFR kinase activity is blocked (FIG. 14E). To verify the utility of selection gene drives against these off-target alterations, a panel of oncogenes was assembled with known or suspected potential to bypass EGFR inhibition. After identifying variants that caused resistance to osimertinib in PC9 cells (FIG. 14F), mixed populations of sensitive, resistant, and gene drive cells were generated. These populations were then treated with osimertinib/dimerizer until the gene drive cells came to dominate, whereupon osimertinib/5-FC treatment was intiated. Across the panel, elimination of these mixed cultures was observed, regardless of the precise source of resistance (FIG. 14G). These results demonstrate the capability of a gene drive circuit to eliminate on-target and bypass resistance mechanisms in a human NSCLC model.

Failure testing against 2,717 mutations in the drug target and 76,441 knockouts across the genome demonstrates the robustness of evolutionary reprogramming in the face of profound heterogeneity.

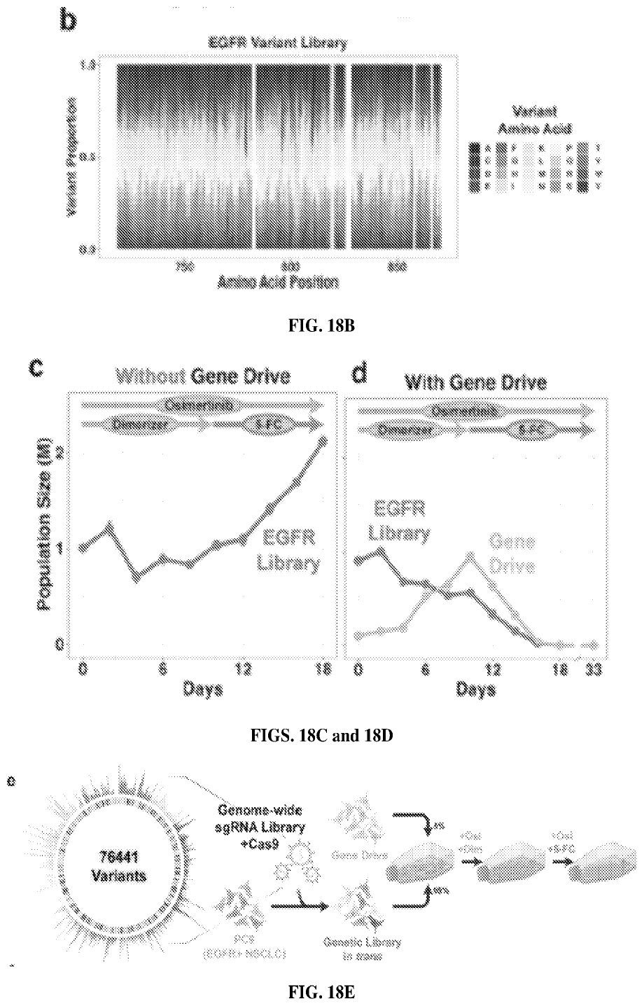

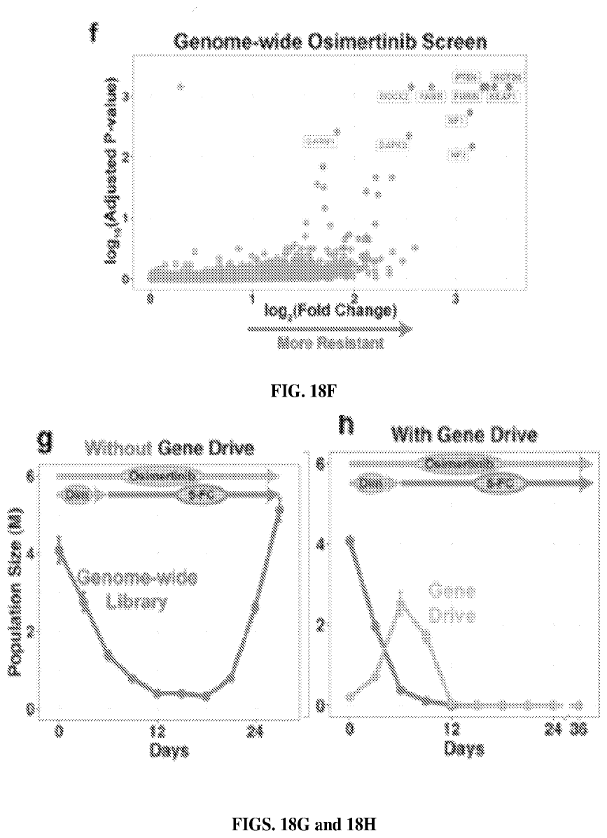

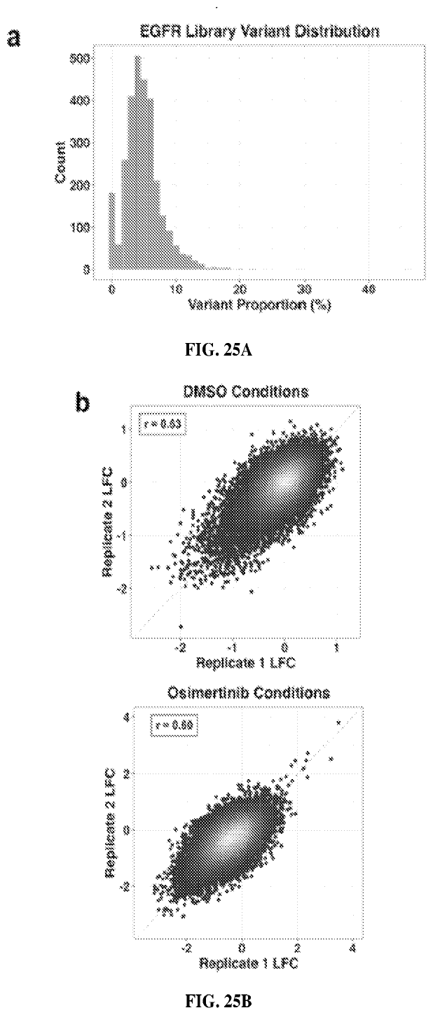

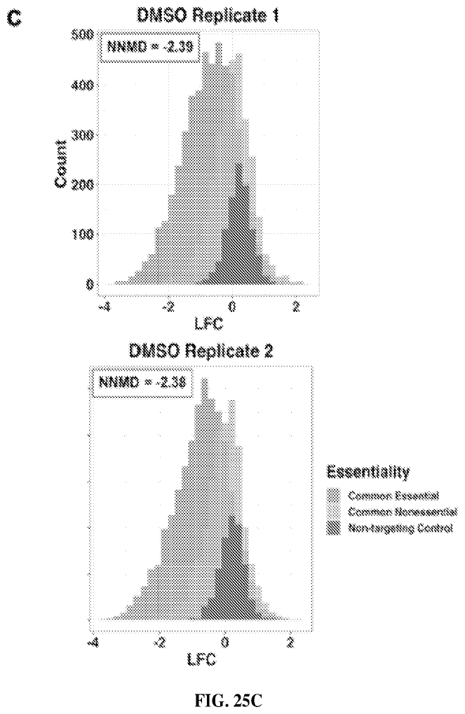

Beyond arrays of spiked-in resistance, it was sought to “stress test” the selection gene drive system in more complex settings. Human cancers are defined by their remarkable heterogeneity, and so to more accurately capture the diversity of clinical tumors, pooled genetic libraries were employed. To assess heterogeneity at the level of the drug target, saturating mutagenesis were used to generate a library of single amino acid substitutions in EGFR L858R (FIG. 18A). The final library comprised of 2,717 variants, spanning 94% of all possible amino acid substitutions along the EGFR kinase domain, with even representation (FIG. 18B and FIG. 25A). Here, a complex population of EGFR variants with a wide range of sensitivities to Osimertinib was prepared. Indeed, PC9 cells transduced with this library exhibited osimertinib resistance after 2-3 weeks of treatment (FIG. 18C). However, a small population of spiked-in gene drive cells was found to outcompete other variants under dimerizer treatment, and then eradicate all cells when 5-FC was administered (FIG. 18D).

These results demonstrate that the selection gene drive system can be agnostic to the exact nature of on-target resistance. In addition to mutations in the target gene and activation of bypass oncogenes, genetic alterations elsewhere in the genome can reshape more distant pathways to promote survival, even in the presence of drug. To assess the gene drive system against these forms of resistance, a genome-wide CRISPR knockout library of 76,441 variants was used to create a diverse population of PC9 cells (FIG. 18E). An osimertinib screen in these cells demonstrated reproducibility and passed standard quality control metrics based on common-essential genes in the untreated conditions (FIGS. 25B, 25C, 25D, and 25E). The drug screen identified a number of resistance-conferring knockouts, including genes involved in RTK/MAPK signaling (e.g. PTEN, NF1/2) and those involved in more distant pathways (e.g. KEAP1, KCTD5) (FIG. 18F). Additional unknown hits for osimertinib resistance, including (e.g. PAWR, CARM1) were identified.

Treating the CRSIPRko PC9 population with osimertinib resulted in an initial decrease in population size, followed by the outgrowth of resistance (FIG. 18G). But in spiking in gene drive cells, it was demonstrated that Switch 1 and Switch 2 treatment could eradicate this extremely heterogeneous population (FIG. 18H). These results demonstrate a gene drive system eliminating genetically diverse cell populations with broad sources of resistance, both within and outside the target gene. Importantly, the engineered heterogeneity used in these experiments is orders of magnitude more diverse than real world tumors.

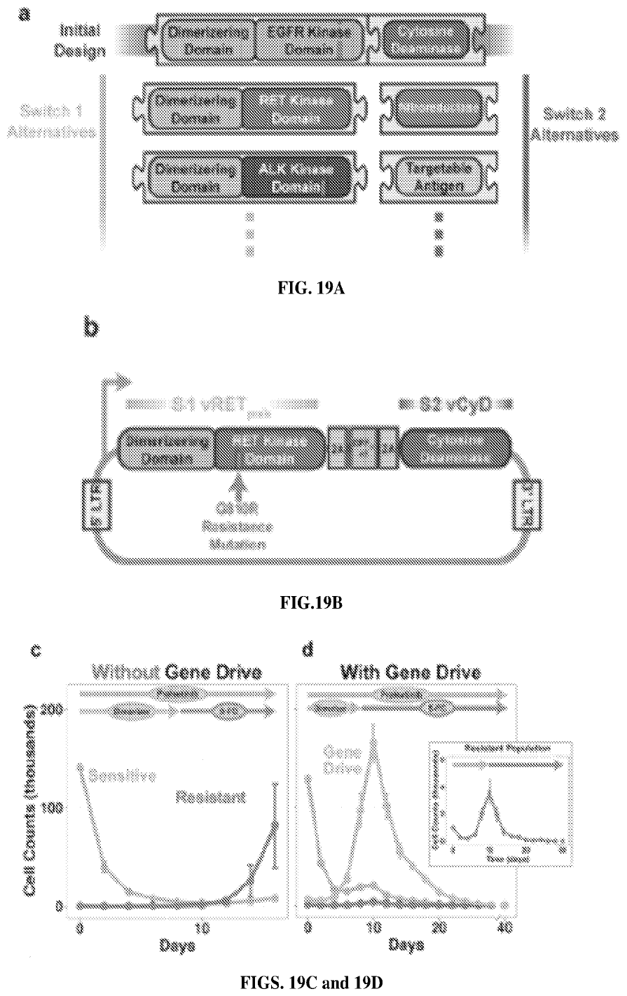

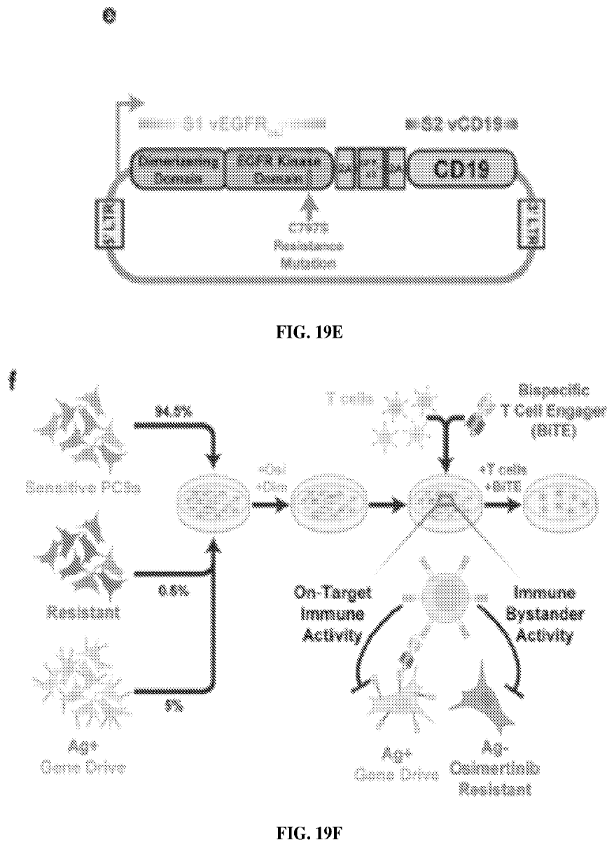

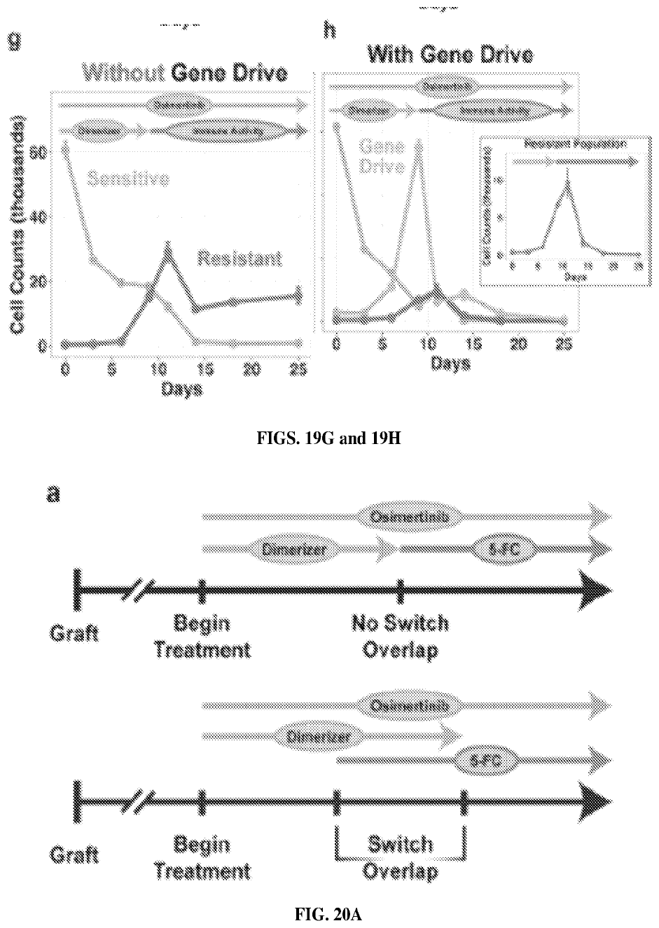

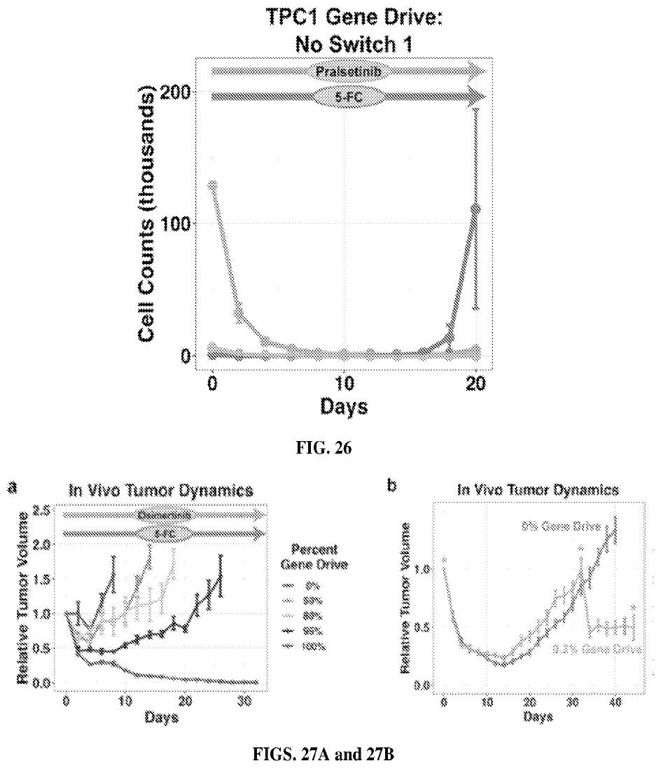

Alternative gene drive systems function in distinct contexts. The selection gene drive system was designed to be a modular platform, with a “plug and play” various Switch 1 and Switch 2 motifs (FIG. 19A). Having subjected the initial S1vEGFRₒₛᵢ-s2vCyD prototype to various genetic stress tests, it was sought to assess the flexibility of the system as a whole by evaluating dual-switch circuits with alternative switch motifs. To determine whether the approach is generalizable to other targets and tissues, the RET version of Switch 1 (S1 vRETprals) and S2 vCyD were cloned into a single genetic construct (FIG. 19B). This system was expressed in RET+ thyroid carcinoma TPC1 cells. In growth tracking experiments for mixed populations, it was found that in the absence of gene drive cells, pralsetinib selected for pre-existing G810R resistance (FIG. 19C). However, when a small gene drive population was spiked in, dimerizer enabled selection of these cells instead. Upon their outgrowth, 5-FC treatment eliminated both gene drive and native resistant populations (FIG. 19D). As in other contexts, gene drive populations required initial Switch 1 selection to clear pre-existing resistance (FIG. 26A). These results show that the selection gene drive approach can achieve evolutionary reprogramming in different lineages and drug targets.

To develop a gene drive system with an orthogonal Switch 2 system, S1 vEGFRₒₛᵢ and S2 vCD19 were cloned into a single vector, generating a complete immune gene drive circuit (FIG. 19E). PC9 cells engineered to express this construct were pooled with sensitive and resistant populations and subjected to osimertinib and dimerizer. Upon outgrowth, T cells and the CD19 bispecific engager blinatumomab were added to initiate anti-CD19 immune activity (FIG. 19F). Resistance emerged in mixed populations lacking gene drive cells, although their complete outgrowth may have been restricted by resource competition with T cells (FIG. 19G). However, pooled populations that included gene drive cells demonstrated sufficient selection of this engineered population to induce strong immune activity that cleared both antigen-positive gene drive cells and antigen-negative, osimertinib-resistant cells (FIG. 19H). Thus, dual-switch genetic circuits with distinct Switch 2 motifs are capable of eliminating pre-existing resistance.

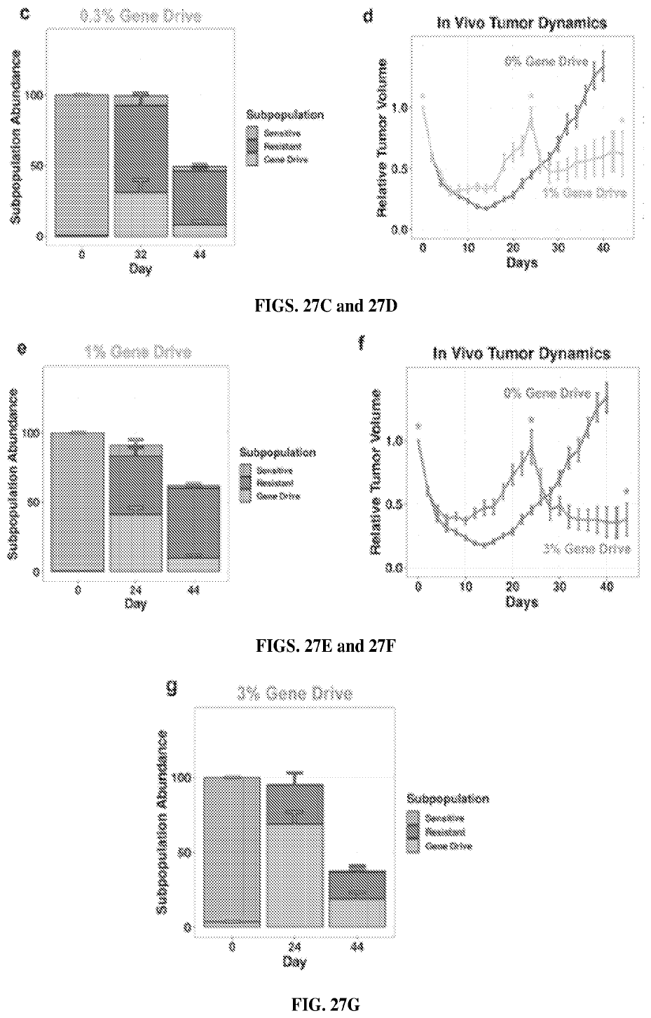

Optimizing switch induction demonstrates in vivo efficacy. Having demonstrated proof-of-concept for diverse gene drive designs in human cancer cells in vitro, it was sought to assess system functionality in vivo. It was noted that the gene drive system exhibited moderately weaker bystander activity in human NSCLC PC9 cells (FIG. 24D) relative to murine BaF3 cells (FIG. 21). Indeed, evaluation of S2 vCyD activity for mixed populations of PC9 cells in vivo indicated that relatively high frequencies of gene drive cells are needed at the beginning of Switch 2 treatment to maximize potency (FIG. 27A). Given these preliminary findings, it was sought to increase efficacy by optimizing switch scheduling.

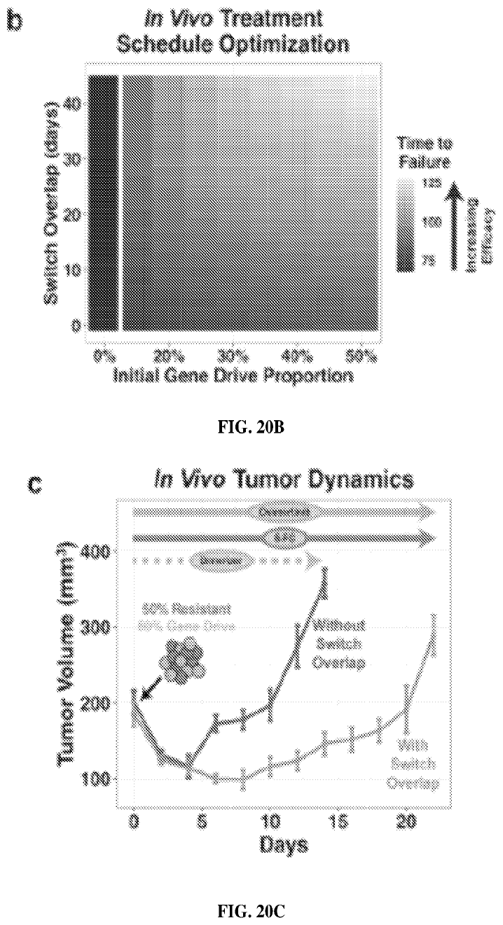

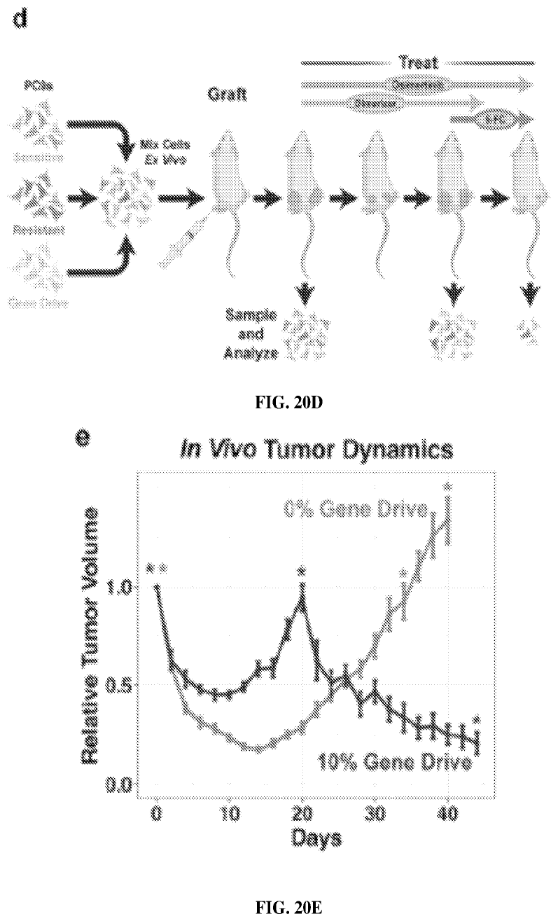

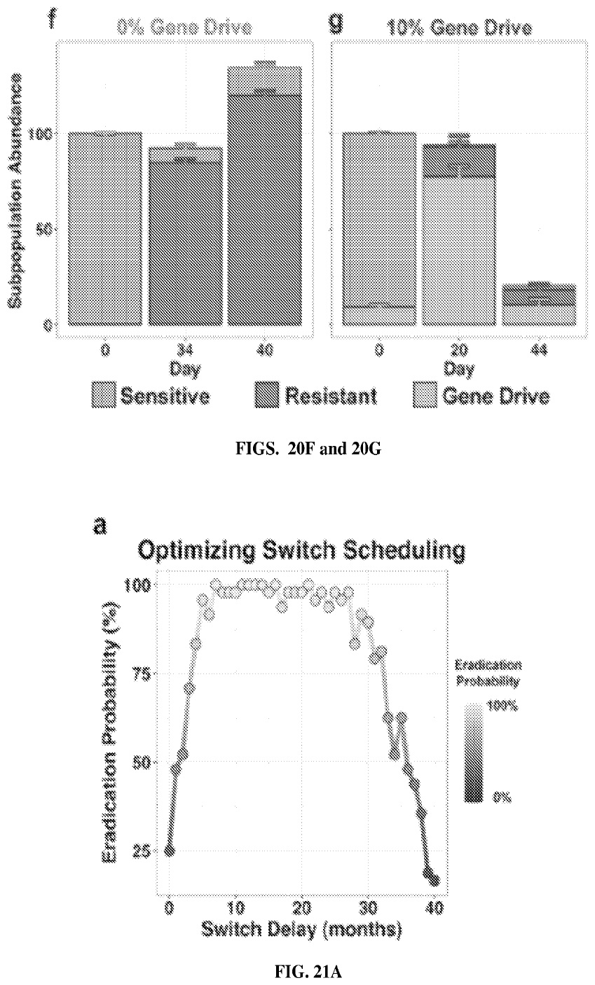

The finding from the evolutionary dynamic models were revisited indicating that there exists some benefit in maintaining Switch 1 treatment for some time after initiating Switch 2 engagement, i.e. switch overlap (FIG. 20A). Thus, the models were reparametrized with growth kinetics for PC9 cells in mice. The model results demonstrate that given the supraphysiological resistance that spiked in pooled populations (orders of magnitude greater than pre-existing resistance in the clinic), complete eradication of these population structures is unlikely. While these high resistance populations were used to reproducibly stress test this approach in vivo, the model did identify regimes where switch scheduling could be optimized to prolong survival in this system. Given that a sufficiently large gene drive population is required for 5-FC activation, the model predicts a benefit in delaying the cessation of Switch 1 in order to avoid rapid clearance of gene drive cells by osimertinib. In doing so, the bystander effect of 5-FU against the osimertinib-resistant population is maximized (FIG. 20B).

To test this, mixtures of 50% gene drive and 50% resistant PC9 cells were generated to reflect a possible population structure at the beginning of Switch 2. These pooled populations were grafted in mice. The mice were then treated with Switch 2 drugs (osimertinib and 5-FC) with or without concurrent Switch 1 engagement (dimerizer). Indeed, tumors receiving temporary dimerizer treatment exhibited a longer time to progression, showing a benefit to overlap in switch scheduling (FIG. 20C).

Finally, these findings were performed in a full-term gene drive experiment in vivo. Pooled populations of sensitive and resistant cells were generated with gene drive spike-in ranging up to 10%, reflecting a gene delivery efficiency that is more conservative than has been clinically demonstrated. These populations were grafted in mice and, upon tumor establishment, treated with the improved switch schedule (FIG. 20D). Tumors without gene drive subpopulations eventually became refractory to osimertinib treatment (FIG. 20E). Analysis of the tumor subpopulations confirmed that the resistant C797S population had driven relapse in these mice (FIG. 20F). However, among mice with spiked-in gene drive cells, these engineered cells came to dominate the tumor population (FIGS. 20E and 20G). The re-engineered tumor was highly sensitized to 5-FC treatment, and subpopulation analyses indicated that the abundance of resistant cells was restricted by bystander activity (FIG. 20G). These findings were reflected in tumors with lower initial gene drive frequencies (FIGS. 27B, 27C, 27D, 27E, 27F, and 27G). Together, these results establish the efficacy of selection gene drive therapy in vivo.

### DISCUSSION

To develop the idea of reprogramming engineered tumors to be more treatable, stochastic models of evolution were used to create a genetic circuit that couples an inducible fitness benefit with a shared fitness defect. This approach was validated by employing synthetic biology techniques to develop genetic constructs capable of leveraging evolutionary principles to eradicate heterogeneous forms of pre-existing resistance.

Initial prototypes were built with repurposed molecular parts and controlled using small molecules that have already been proven to be safe in humans. Beyond these designs, it was illustrated that the modularity of selection gene drive motifs. Alternative Switch 1 designs demonstrated inducible fitness benefits across different drugs and tumor types. Orthogonal Switch 2 systems, including an immune-mediated anticancer mechanism, exhibited strong bystander activity. To demonstrate the evolutionary robustness of this approach, selection gene drives were shown to eradicate astonishing levels of genetic heterogeneity within a drug target and across the genome. Finally, evolutionary models were employed to optimize the dynamics of switch engagement in vivo. Importantly, all in vitro and in vivo experiments were performed in the presence of a large, supraphysiological population of pre-existing resistant cells. Thus, this design demonstrates the establishment of evolutionary control over a tumor cell population otherwise destined for treatment failure.

Selection gene drive technology builds upon previous advancements in the emerging field of evolutionary therapy. The practice of adaptive therapy uses evolutionary principles to inform drug dosing and/or scheduling to maintain a residual sensitive tumor cell population that suppresses the outgrowth of resistance, rather than a maximum tolerated dosing regimen that enables the competitive release of resistant subclones. A recent phase II clinical study of adaptive therapy in prostate cancer reported promising results. Similarly, the Switch 1 phase of selection gene drive treatment involves careful control of a population that acts to restrain resistance outgrowth, through competition for resources and space.

However, gene drive therapy expands upon adaptive therapy by employing not just passive suppression of resistance variants, but active killing through Switch 2 bystander activity. Additionally, gene drive therapy does not assume a fitness cost among resistance populations and succeeds even when gene drive cells are less fit than native resistance (FIG. 1F).

Another evolutionary-informed therapeutic approach involves exploiting collateral sensitivities to set “evolutionary traps”. As in gene drive therapy, treatment strategies that leverage collateral sensitivity use sequences of drugs to guide the evolutionary trajectories towards favorable outcomes. Under collateral sensitivity, administration of one drug selects for a tumor population that is sensitive to a second drug. However, natural forms of collateral sensitivity are likely to be uncommon. Rather than relying on native collateral sensitivity, gene drive therapy engineers a genetic vulnerability (Switch 2) directly into the redesigned tumor.

Additionally, leveraging natural collateral sensitivity requires that tumors reliably follow an expected evolutionary trajectory. Under a selection gene drive approach, Switch 1 provides a strong selection effect to reproducibly control evolution, and Switch 2 bystander activity enables the targeting of subpopulations that do not harbor the secondary genetic vulnerability directly.

There remain a number of practical considerations towards the successful translation of selection gene drives. Chief among these is delivery. Tumor cells can be modified in situ to express the genetic circuit. Undoubtedly, a safe gene drive therapy approach requires the specific delivery and expression of the genetic switches. Progress in the targeted delivery of nucleic acids and tumor specific gene expression highlights this approach. Alternatively, tumor cells can be modified ex vivo and reintroduced, leveraging the capability of circulating cancer cells to home to tumor niches.

Regardless of the specific delivery mechanism, by leveraging the power of selection, even a small, modified cell population is sufficient to achieve favorable outcomes. Thus, a safe expression profile over a maximally efficient delivery system is achieved. Moreover, decades of research in tumorigenesis show a high intrinsic barrier to transformation among normal mammalian cells, as expression of activated oncogenes without the loss of a tumor suppressor has been shown to induce senescence. This differential selective effect between cancer and normal cells provides a dramatic therapeutic window for this approach, even in the absence of targeted delivery or expression restriction. Thus, even a modest tumor selectivity for gene drive delivery and expression exploits this tumor-specific selection to maximize safety.

In this example, tumors are re-engineered to be more responsive to therapeutic intervention. Initial selection gene drive designs are feasible; they behave accordingly and are robust in the face of dramatic genetic and spatial failure modes. While the gene drive approach has risks, the intractability of treatment of late-stage tumors and the dramatic genetic diversity present in tumors at baseline necessitates bold new approaches. By leveraging evolutionary theory, tumors are reprogrammed to reliably and effectively target heterogeneity.

### Methods

Description of compartmental dynamic model. In the stochastic model of tumor evolutionary dynamics, an initial population of 100 drug-sensitive cells that expands according to a birth-death-mutation process was expanded. Mutation events spawn subclones resistant to therapy at a mutation rate u. Once the cancer cell population reaches a predetermined detection size M, a fraction (q) of cells are “infected” and assigned gene drive specific parameters. At the same time, targeted therapy is initiated and Switch 1 is engaged. During the Switch 1 phase of treatment, gene drive cells retain a positive net growth rate until they reach population size M, whereupon Switch 2 is engaged.

In total, eight populations are modeled, including sensitive wild-type cells, cells resistant to targeted therapy, cells resistant to Switch 2 killing, and cross-resistant cells insensitive to both forms of therapy. In addition, the model considers gene drive cells, as well as those with acquired resistance to either or both forms of therapy (FIG. 1D).

Birth, death, and drug-sensitive drug kill rates are 0.14, 0.13, and 0.04/day. Resistant subpopulations are completely insensitive to drug killing. The bystander effect of Switch 2 activity was modeled by scaling the drug kill rate by the proportion of tumor cells that express the Switch 2 gene. Tumor detection size (M), mutation rate (μ), gene delivery efficiency (q), and the net growth rate of gene drive cells during Switch 1 (g_{gd}) are allowed to vary. Tumor detection sizes ranged from 10⁸ to 10¹² cells; mutation rates ranged from 10⁻⁹ to 10⁻⁶/division; gene delivery efficiency ranged from 0.1% to 30%; gene drive Switch 1 growth rate varied from 0.01 (completely resistant) to 0.0044/day.

The system was solved stochastically using a modified Gillespie algorithm with adaptive tau leaping using MATLAB. Each combination of parameters was simulated 48 times. The simulation code is available on GitHub.

Description of spatial agent-based model. In the spatial agent-based model, mixed populations of tumor cells (10⁴ cells, including 0.5% resistant and 5% gene drive) are assigned positions in 3D space. The initial spatial positions of resistant cells are randomly selected, but gene drive cells are centered at a random focus. The position of each gene drive cell is drawn from an exponential distribution weighted by distance from the focus and a dispersion parameter (γ). Thus, when the dispersion parameter is low, gene drive cells are concentrated around the focus. Alternatively, when the dispersion parameter is large, positioning is effectively random and gene drive cells are evenly seeded.

After seeding, the cells follow a birth-death process, with dividing cells “budging” their neighbors to create space. Initially, targeted therapy and Switch 1 treatment is maintained. Once the gene drive population dominates the tumor, Switch 2 is engaged. To model the spatial range of bystander activity, a “kill radius” parameter (p) is assigned. Any cell within p cell lengths of a gene drive cell is considered “adjacent” and is subject to Switch 2 bystander activity. Each parameter set is simulated for 25 virtual tumors. The simulation continues until the entire population is eradicated or gene drive cells are exhausted. Simulation code is available on GitHub.

Construct generation. PCR-based cloning was used to insert genes of interest (including EGFR L858R, cytosine deaminase, and CD19) into the pLVX-IRES-Puro vector (Addgene). Switch 1 constructs were similarly generated by cloning target kinase domains into the pL VX-Hom-Mem1 vector (Takara). Site-directed mutagenesis was used to generate resistance variants. Proper assembly and mutation identity was confirmed by Sanger sequencing.

Cell culture. BaF3 (DSMZ), PC9 (Sigma Aldrich), TPC1 (Sigma), HCC78 (DSMZ), and H3122 (NCI) cells were maintained in RPMI-1640 (Sigma Aldrich)+10% FBS (Corning)+1% penicillin/streptomycin (Life Technologies). Before transformation, BaF3 cells were cultured in 10 ng/ml murine IL-3 (PeproTech). Cells were grown in a 37C incubator with 5% CO2.

Early passage wild-type PC9 cells exhibited initial regression followed by rapid outgrowth in erlotinib and osimertinib, even at high concentrations, suggesting a substantial pre-existing resistance subpopulation. To develop a clean PC9 population with reproducible drug response, we isolated an EGFRi-sensitive clone. This monoclonal line served as the wild-type, sensitive population in PC9 experiments and was used to generate gene drive and resistant PC9 cells.

Lentiviral transduction. pLVX constructs were co-transfected with third-generation lentiviral packaging plasmids and VSV-G in HEK293T cells (ATCC) using calcium phosphate. The viral supernatant was collected at 48 hours and used to infect the target cell. To generate fluorescently labeled BaF3 cells used in growth tracking experiments, multiple sequential rounds of infection and selection were relied upon. For gene drive cells, BaF3s were infected with pLVX-Puro-IRES-GFP (Addgene), selected on puromycin, infected with pLVX-EGFR_L858R-IRES-Puro, selected on IL-3 independence, infected with pLVX-Hom-Mem1-EGFR, and finally selected on erlotinib and dimerizer. For resistant cells, BaF3s were infected with Hyg-2A-mCherry (Addgene), selected on hygromycin, infected with pLVX-EGFR_L858R/T790M-IRES-Puro, and selected on puromycin.

Similar sequential infections and selections were used to generate fluorescently-labeled resistant cells in the PC9 and TPC1 systems. To generate PC9 and TPC1 gene drive cells, cells were first infected with GFP1-10-IRES-Puro (Addgene) and selected on puromycin. These cells were then infected with the appropriate gene drive construct containing a short GFP11 sequence. Gene drive cells were then sorted by FACS for reconstituted GFP.

BaF3 dimerizer-dependence assays. S1 vEGFRₑᵣₗ BaF3s were seeded in 12-well plates at 100k/well with dimerizer (AP20187, Takara) in triplicate. Cell counts were measured on a hemocytometer every day for four days, and an exponential curve was fit to the data to estimate growth rates.

Engineered Switch 1 BaF3 in vivo models. All animal experiments were conducted under a protocol approved by the Institutional Animal Care and Use Committee. In the dimerizer-dependence in vivo experiment, S1 vEGFRₑᵣₗ BaF3 cells were subcutaneously grafted on both flanks (3.5M/flank) of NOD-SCID mice (Jackson Labs). Mice were randomized into four arms (0, 0.1, 1, and 10 mg/kg dimerizer) of 3 mice each. Mice received 100 uL dimerizer or vehicle control (2% Tween in PBS) once daily via intraperitoneal injection. Tumor volumes were measured with calipers following 12 days of treatment.

Drug dose response assays. In general, all ₁C50 measurements were conducted similarly. Cells were seeded in 96-well plates at 3k/well triplicate. Adherent cells were given 24 hours before the addition of drug. Cell viability was measured three days after drug treatment using CellTiter-Glo 2.0 (Promega) and luminescence values were normalized to vehicle control conditions.

Immunoblotting. PC9 cells were seeded at 250k/well in 12-well plates. After 24 hours, 250 nM erlotinib and/or 10 nM dimerizer was added. Four hours after drug treatment, cells were lysed on ice (LDS NuPage Buffer and Reducing Agent) and stored in-80C. Cell lysates were subjected to western blotting using the indicated primary antibodies (p-EGFR, p-ERK, p-Akt, CellSignaling) and HRP-conjugated antibody (CellSignaling). Signal was visualized with SuperSignal Chemiluminescent substrate reagent (ThermoFisher) on a BioRad imager.

In vivo cytosine deaminase activity. Mice were randomized into three arms (five mice/arm). EGFR BaF3 cells that did (two arms) or did not (one arm) express S2 vCyD were subcutaneously grafted in both flanks of the mice.

Tumors were allowed to grow for 12 days, and then once-daily treatment was initiated. The wild-type arm and one of the S2 vCyD arms received 800 uL 500 mg/kg 5-FC via ip injection. The second S2 vCyD arm received 800 uL vehicle control (sterile PBS). Tumor volumes were measured every other day.

Enzyme-prodrug bystander assays (vCyD and vNfsA). To evaluate the S2 vCyD system, populations of wild-type and cytosine deaminase-expressing BaF3 cells were mixed at defined ratios and seeded at 30k/well in 96-well plates in triplicate, and 1 mM 5-FC was added. Cell viability was measured after 48 hours using CellTiter-Glo and normalized to untreated controls. Similarly, wild-type and S1vEGFRₒₛᵢ-S2vCyD PC9 cells were mixed, seeded at 20k/well, and treated with 1 mM 5-FC.

To evaluate the S2 vNfsA system, populations of wild-type and NfsA-expressing 293T cells were mixed, seeded at 20k/well in 96-well plates in triplicate, and treated with 100 μM CB1954. Cell viability was measured after 24 hours using CellTiter-Glo and normalized to untreated controls.

Immune bystander assays (vCD19). PBMCs were sourced from Astarte and T cells were expanded using CD3/CD28 dynabeads (1:100 bead: cell ratio). For the immune bystander experiments, CD19+ and CD19-PC9 cells were seeded 1:1 in 24 wells plates at 60k/well. After 24 hours, 1 ng/mL blinatumomab was added to all wells. At the same time, freshly-thawed T cells were added at the appropriate concentration. Each target: effector ratio was conducted in triplicate. After 48 hours, the supernatant and resuspended adherent cells were pooled and analyzed according to the following staining protocol. Cells were spun down (750 g for 3 min) and resuspended in 50 uL Fc block buffer. After a 10 minute incubation, 100 uL antibody mixture (anti-CD3/FITC and anti-CD19/APC) was added. The cell suspension was incubated at 4C for 20 minutes. After three washes in PBS with 1% BSA, the cell suspension was analyzed by flow cytometry.

In the transwell version of the experiment, CD19+ and CD19-cells (30k/well each) were seeded either together in the bottom of a transwell plate or separated with CD19-cells in the bottom well. After 24 hours, 1 ng/mL blinatumomab was added to each well, in addition to 120k T cells (2:1 effector: target ratio). Each condition was run in triplicate. After 48 hours, cell suspensions were recovered and analyzed as above.

Gene drive growth tracking. Populations of sensitive, mCherry+ resistant, and GFP+ gene drive cells were mixed together. Except where otherwise noted, mixed populations consisted of 0.5% resistant cells and 5% gene drive cells. Cells were seeded in 24-well plates (1.5M/well for BaF3s and 50k/well for adherent cells) in triplicate, with the exception of the gene-drive titer experiment where 3M/well BaF3s were seeded in 6-well plates in order to maintain sufficient cell numbers for low spike-in conditions. For Switch 1 conditions, 10 nM dimerizer and 250 nM erlotinib, 50 nM osimertinib, or 1 μM pralsetinib were used. For Switch 2 conditions, 500 μM 5-FC replaced dimerizer in the above formulations.

Cell counts were measured every other day. For counts of adherent cells, wells were washed with PBS, trypsinized, and then resuspended in RPMI. Cell suspensions were transferred to microcentrifuge tubes, vortexed, and a small aliquot (6%) was analyzed by flow cytometry (BD Accuri) to get subpopulation cell counts. The remaining cells were spun down (1k g for 5 min), the supernatant was aspirated, and the cell pellet was resuspended in fresh RPMI and seeded onto a new plate. Fresh drug was added immediately. In general, Switch 2 treatment began when the gene drive population exceeded 60% of the day 0 cell counts. Cells were monitored for 2-3 weeks after apparent eradication to ensure that no remaining cells grew back.

EGFR variant library. The EGFR single-site variant library was synthesized and cloned by Twist Bioscience. In brief, saturating mutagenesis was used to introduce all possible amino acid substitutions (optimized for H. sapiens codon bias) between L718 and H870 residues (with the exception of R858) in the EGFR L858R kinase domain. Large-scale bacterial transformation maintained>2000-fold library coverage. Lentivirus was prepared as above and stored at −80C. A test infection in PC9s with polybrene (4 ug/mL) was used to estimate the viral titer. The large-scale infection of PC9s maintained 450-fold post-selection library coverage, with a 5% infection efficiency to ensure low MOI. 1M cells (330-fold library coverage) were seeded in 6-well plates in triplicate. In the gene drive conditions, gene drive cells were spiked in at 10% abundance. Switch 1 and Switch 2 formulations were prepared as previously. Cell counts were measured every other day by flow cytometry, and fresh drug was prepared for each time point.

Genome-wide osimertinib screen. The genome-wide Brunello CRISPR knockout library was ordered from Addgene. Lentivirus was prepared as above and stored at −80C, and a small-scale infection was used to assess infection efficiency in PC9s. PC9 cells were infected in two large-scale replicates at 200-fold post-selection library coverage, with a 5-10% infection efficiency.

For the osimertinib drug screen, the two infection replicates were divided into osimertinib and untreated populations. Each condition was seeded at 300M cells (390-fold library coverage) and treated with either 10 nM osimertinib or the equivalent volume of DMSO. Cells were subcultured every three days to maintain high library coverage (>250-fold). After 15 days, the cell pellets were harvested and frozen.

gDNA was extracted from cell pellets using the Qiagen maxi kit. sgRNAs were amplified using Illumina PCR primers and sequenced on a HiSeq 3000. Guide counts were quantified using the Broad Institute GPP's PoolQ pipeline, with the default settings. Osimertinib enrichment/depletion was determined by counting log-fold changes and adjusted p-values, as calculated by the MAGeCK algorithm. Raw data and analysis code is available on GitHub.

For the pooled CRISPRko gene drive experiments, fresh PC9 cells were infected with the Brunello library in duplicate at 150-fold coverage. After selection, the two infection replicates were seeded in 10 cm dishes at 4M cells/plate (50-fold coverage). In the gene drive conditions, gene drive cells were spiked in at 5% frequency. Switch 1 and Switch 2 formulations were prepared as in other growth tracking experiments. Cell counts were measured every three days by flow cytometry, and fresh drug was added at each time point.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the disclosure being indicated by the following claims.

1. A nucleic acid composition comprising a fitness benefit molecule, a fitness cost gene, and a promoter within a pharmaceutically acceptable carrier, wherein the fitness benefit molecule comprises a dimerization domain gene operably linked to a drug resistant gene.

2. The composition of claim 1, wherein the fitness benefit molecule comprises a resistance gene, metabolite, a growth factor, a cytokine, a supplement, or a biomolecule thereof.

3. (canceled)

4. The composition of claim 1, wherein the fitness cost gene is a suicide gene.

5. (canceled)

6. The composition of claim 1, wherein the fitness cost gene is located downstream of the fitness benefit gene.

7. The composition of claim 1, wherein the dimerization domain gene encodes a dimerizing protein.

8. The composition of claim 1, wherein the drug resistant gene encodes a drug resistant receptor.

9. The composition of claim 1, wherein the dimerizing protein is fused to the drug resistant receptor.

10. The composition of claim 1, wherein the drug resistant receptor is a drug resistant tyrosine kinase receptor.

11. The composition of claim 1, wherein the suicide gene encodes a suicide enzyme.

12. (canceled)

13. (canceled)

14. A gene selection drive system comprising the nucleic acid composition of claim 1, wherein said system is activated in a cell population via a dimerizer and a therapeutic compound.

15. (canceled)

23. The system of claim 14, wherein the suicide enzyme converts a prodrug into an active drug.

24. (canceled)

25. The system of claim 14, wherein the active drug kills a residual cell not comprising the system.

26. The system of claim 14, wherein the dimerizer is a peptide, polypeptide, or small molecule.

27. The system of claim 14, wherein the dimerizer is a FK506-binding protein 12 (FKBP12) peptide.

28. The system of claim 14, wherein the active drug is a chemotherapy drug.

29. A cell comprising the gene selection drive system or nucleic acid composition of claim 14.

30. A method of treating or preventing a cancer in a subject in need thereof, the method comprising a gene selection drive system or a nucleic acid composition further comprising a fitness benefit molecule, a fitness cost gene, and a promoter within a pharmaceutically acceptable carrier, wherein the resistance gene comprises a dimerization domain gene operably linked to a drug resistant target gene.

31. The method of claim 30, wherein the system is activated in a tumor of the subject when a dimerizer and a therapeutic compound are further administered simultaneously or individually.

32. The method of claim 30, wherein the dimerizer interacts with one or more dimerizing proteins fused to a drug resistant receptor to induce drug resistance in the tumor.

33. The method of claim 30, wherein the fitness benefit molecule promotes cell growth in the subject.

34. The method of claim 30, wherein the fitness cost gene encodes a suicide enzyme whereby said suicide enzyme converts a prodrug into an active chemotherapeutic drug.

35. The method of claim 30, wherein the fitness cost gene is a cytosine deaminase gene, a NADPH nitroreductase gene, or a diptheria toxin gene.

36. (canceled)

37. The method of claim 30, wherein the dimerizer is a FK506-binding protein 12 (FKBP12) peptide.

38. The method of claim 30, wherein the therapeutic compound and the active chemotherapeutic drug kill at least 80% of cancer cells in the tumor.

39-41. (canceled)


SEQUENCE LISTING (14 sequences)

SEQ ID NO: 1 - protein, 19 aa, Homo sapiens
YSMKKTTMKIIPFNRLTIG

SEQ ID NO: 2 - protein, 4 aa, Homo sapiens
DGEA

SEQ ID NO: 3 - protein, 8 aa, Homo sapiens
PHSRNRGD

SEQ ID NO: 4 - protein, 6 aa, Homo sapiens
GROGER

SEQ ID NO: 5 - protein, 5 aa, Homo sapiens
IKVAV

SEQ ID NO: 6 - protein, 5 aa, Homo sapiens
GRKRK

SEQ ID NO: 7 - protein, 6 aa, Homo sapiens
FYFDLR

SEQ ID NO: 8 - protein, 8 aa, Homo sapiens
IPVSLRSG

SEQ ID NO: 9 - protein, 8 aa, Homo sapiens
RPFSMIMG

SEQ ID NO: 10 - protein, 8 aa, Homo sapiens
VPLSLTMG

SEQ ID NO: 11 - protein, 8 aa, Homo sapiens
VPLSLYSG

SEQ ID NO: 12 - protein, 8 aa, Homo sapiens
GPLGLWAR

SEQ ID NO: 13 - protein, 8 aa, Homo sapiens
IPESLRAG

SEQ ID NO: 14 - protein, 8 aa, Homo sapiens
VPMSMRGG